(19) United States
(12) Patent Application Publication
(10) Pub. No.: US 2022/0370624 A1
(43) Pub. Date: Nov. 24, 2022

(54) LIPID COMPOSITIONS COMPRISING PEPTIDE-LIPID CONJUGATES

(71) Applicant: Arcturus Therapeutics, Inc., San Diego, CA (US)

(72) Inventors: Kumar Rajappan, San Diego, CA (US); Steven Tanis, San Diego, CA (US); Rajesh Mukthavaram, San Diego, CA (US); Amit Sagi, San Diego, CA (US); Priya Prakash Karmali, San Diego, CA (US); Padmanabh Chivukula, San Diego, CA (US)

(21) Appl. No.: 17/737,884

(22) Filed: May 5, 2022

Related U.S. Application Data
(60) Provisional application No. 63/184,584, filed on May 5, 2021.

(51) Int. Cl. A61K 47/54; A61K 47/26; C07K 7/04; C07K 14/00; C07K 19/00; A61P 35/00; A61K 9/00

(52) U.S. Cl. CPC A61K 47/543; A61K 47/26; C07K 7/04; C07K 14/001; C07K 19/00; A61P 35/00; A61K 9/0019

(57) ABSTRACT

A lipid composition containing a nucleic acid, wherein the lipid composition comprises a peptide-lipid conjugate, is provided. The peptide of the peptide-lipid conjugates can be from 4 to 52 amino acids in length. Methods of using the lipid composition in the in vivo delivery of nucleic acids are further provided.

### CROSS-REFERENCE TO RELATED APPLICATIONS

This application claims priority to U.S. Provisional Application No. 63/184,584, filed May 5, 2021, which is hereby incorporated by reference in its entirety and for all purposes.

### TECHNICAL FIELD

The present disclosure relates to lipid conjugates. More specifically, the present disclosure relates to peptide-lipid conjugates useful in lipid delivery technology.

### BACKGROUND

Delivery of therapeutic agents into the cells or tissues of human subject is important for its therapeutic effects and is usually impeded by a limited ability of the compound to reach targeted cells and tissues. Many macromolecules and molecules with net ionic charges face multiple hurdles in entering cells, and the problem becomes even more complicated when such drugs have to be delivered to specific cell types of interest. Unlike small molecule drugs, these types of molecules do not undergo passive diffusion across a cell membrane. Biologically active proteins such as immunoglobulins and potential therapeutics of the polynucleotide class, such as genomic DNA, cDNA, mRNA, and siRNA, antisense oligonucleotides, and even certain low molecular weight peptides, peptide hormones and antibiotics are some of the examples of biologically active molecules for which effective targeting to a patient's tissues is often not achieved.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the United States Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics via lipid formulations is still undergoing development.

Lipid-based formulations often have a polyethylene-glycol (PEG) based compound as one of the components. The PEG can be conjugated to a lipid, cholesterol, a cationic-polymer, or other compounds to facilitate integration into the lipid-based formulation. Typically, the PEG is included in a lipid formulation as a coating or surface ligand, a technique referred to as PEGylation, which helps prevent the aggregation of lipid particles, liposomes, micelles, etc. and to protect the lipid-based formulations from the immune system and their escape from reticuloendothelial (RES) uptake (Nanomedicine (Lond). 2011 June; 6(4):715-28). PEGylation has been widely used to stabilize lipid formulations and their payloads through physical, chemical, and biological mechanisms. Detergent-like PEG lipids (e.g., PEG-DSPE) can enter the lipid formulation to form a hydrated layer and steric barrier on the surface. Based on the degree of PEGylation, the surface layer can be generally divided into two types, brush-like and mushroom-like layers. It has been shown that increased PEGylation leads to a significant increase in the circulation half-life of lipid formulations (Annu. Rev. Biomed. Eng. 2011 Aug. 15; 13:507-30; J. Control Release. 2010 Aug. 3; 145(3):178-81).

Despite the benefits and uses of PEG-conjugates in lipid-based formulations, the use of PEG has also been associated with several problems. For example, studies on the intracellular delivery of nucleic acids by Song et al. found that PEG-lipids severely inhibited active nucleic acid transfer and the endosomal release of antisense oligodeoxynucleotides into the cytoplasm (Song, L. Y., et al. Biochimica et Biophysica Acta (BBA)-Biomembranes. 2002 1558(1):1-13). Additionally, PEG as a molecule not naturally found in living systems has been associated with undesired immunogenic responses (Garay and Labaune. The Open Conference Proceedings Journal. Vol. 2. No. 1. 2011). After decades of using PEGylated drugs in human therapeutics, it has been observed that treating patients with PEGylated drugs can lead to the formation of antibodies that specifically recognize and bind to PEG (anti-PEG antibodies). Anti-PEG antibodies are also found in patient who have never been treated with PEGylated drugs but have consumed products containing PEG (Hoang Thi et al. Polymers 12(2):298. 2020). Thus, treating patients who produce anti-PEG antibodies with PEGylated drugs results in accelerated blood clearance, low drug efficacy, hypersensitivity, and in some cases, life-threatening side effects.

Several alternative polymers have been investigated as potential replacements for PEGylation in pharmaceutical compositions. Some of these include the investigation of hydrophilic polymers such as polyoxazolines, poly(N-vinylpyrrolidone), poly(glycerols), and polyacrylamides; natural polymers such as lipids, carbohydrates, and proteins (e.g., serum albumin), and polyaminoacids; or zwitterionic polymers such as poly(carboxybetaine), poly(sulfobetaine), and phosphobetaine-based polymers (Hoang Thi et al. 2011). Many of these polymers are found in daily products or other pharmaceutical compositions and run the risk of creating immunogenic responses. One protein that has gained some interest is the XTEN peptide technology, which has been utilized in peptide sizes of 144, 288, 432, 576, and 864 amino acid residues in length to fuse to therapeutic peptides and proteins ton increase in vivo half-life (Podust et al. Journal of Controlled Release 240 (2016): 52-66). While significant developments have been made in finding alternatives for PEGylated compositions, XTEN and the other tested polymers have mainly been characterized in their ability to increase in vivo half-life and tend to be too large for nucleic-acid lipid deliver applications. Furthermore, any PEGylation alternative for nucleic acid lipid delivery must be able to conjugate with suitable lipids that can achieve not only a desirable in vivo half-life, but also target cell uptake, and acceptable shedding rates from the lipid formulation. Thus, new PEG alternatives are needed that are specifically suitable to the unique needs of nucleic acid lipid delivery compositions.

### SUMMARY

Additional features and advantages of the subject technology will be set forth in the description below, and in part will be apparent from the description, or may be learned by practice of the subject technology. The advantages of the subject technology will be realized and attained by the structures particularly pointed out in the written description and embodiments hereof as well as the appended drawings.

It is to be understood that both the foregoing general description and the following detailed description are exemplary and explanatory and are intended to provide further explanation of the subject technology.

The present disclosure provides compositions of peptide, peptide mimetics and their conjugates that can be used in the formulation of lipid formulations encapsulating drug molecules, including oligonucleotide drugs, such as ribonucleic acids and deoxy-ribonucleic acids. These peptide, peptide mimetics, and their conjugates can show superior ability over PEG-conjugates in the delivery of nucleic acid therapeutics in vivo. The peptides may comprise repeating units of serine, threonine, glutamic acid and proline as tetrapeptides (STEP peptides), although such repeating sequences are not the only arrangement that can achieved the result of superior ability of PEG-lipid conjugates. Specifically, the inclusion of threshold amounts of hydrophilic amino acids or derivatives thereof can potentially form a “water cage” around a lipid particle through hydrogen bond interactions with the amino acid side chains. Furthermore, the inclusion of a threshold amount of proline, an amino acid that lends rigidity to a peptide's structure, further provides favorable conformation of the peptide portion of the peptide-lipid conjugate that can promote the formation of the “water cage”. Such a layer of water is likely to act as a steric barrier against interaction of LNPs with blood components and prevent opsonization, complement activation and premature clearance while the LNP is in circulation. Additionally, with this approach of peptide conjugation and formulation techniques various peptides with tunable properties can be incorporated in an LNP matrix so that its functional, cell and tissue specificity, and pharmacokinetic and toxicological properties can be modulated to meet the requirements of different applications.

In an embodiment, a lipid composition is provided containing a nucleic acid, wherein the lipid composition comprises a peptide-lipid conjugate.

In an embodiment, a lipid composition is provided comprising a peptide-lipid conjugate and a nucleic acid, wherein:
i. the peptide of the peptide-lipid conjugate consists of about 4 to about 52 amino acids;
ii. at least about 14% of the amino acids in the peptide of the peptide-lipid conjugate are proline;
iii. about 28% to about 80% of the amino acids in the peptide of the peptide-lipid conjugate have a hydrophilic side chain;
iv. less than about 43% of the amino acids in the peptide of the peptide-lipid conjugate are glycine;
v. the lipid composition comprises one or more cationic lipids, one or more helper lipids, and a sterol; and
vi. the peptide-lipid conjugate comprises about 0.1 mol % to about 10 mol % of all lipids in the lipid composition.

In some embodiments, methods are provided for delivering a lipid composition of the present disclosure to a target. In some embodiments, methods of treating a disease in a subject are provided comprising administering a lipid composition of the present disclosure.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 shows the effect of using peptide-lipid conjugates described herein in lipid nanoparticle formulations as compared to PEG on the in vivo expression of human erythropoietin (hEPO) expression levels (ng/ml) as described in Example 7.

FIG. 2 shows the effect of using peptide-lipid conjugates described herein in lipid nanoparticle formulations as compared to PEG on the in vivo knockdown of Factor VII (FVII) normalized to phosphate buffered saline (PBS) baseline as described in Example 8.

FIG. 3 shows representative images of liver and spleen sections stained for detection of tdTomato protein expression. mRNA allowing for tdTomato expression was delivered to the organs by injection of lipid nanoparticle (LNP) formulations including Peptide 7 or DMG-PEG conjugate, as described in Example 5.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

In an embodiment, a lipid composition is provided containing a nucleic acid, wherein the lipid composition comprises a peptide-lipid conjugate.

In some embodiments, the peptide of the peptide-lipid conjugate consists of about 4 to about 52 amino acids. In some embodiments, the peptide of the peptide-lipid conjugate is about 8 to about 52 amino acids in length. In some embodiments, the peptide of the peptide-lipid conjugate is about 12 to about 52 amino acids in length. In some embodiments, the peptide of the peptide-lipid conjugate is about 16 to about 52 amino acids in length. In some embodiments, the peptide of the peptide-lipid conjugate is about 20 to about 52 amino acids in length. In some embodiments, the peptide of the peptide-lipid conjugate is about 24 to about 52 amino acids in length. In some embodiments, the peptide of the peptide-lipid conjugate is about 28 to about 52 amino acids in length. In some embodiments, the peptide of the peptide-lipid conjugate is about 32 to about 52 amino acids in length. In some embodiments, the peptide of the peptide-lipid conjugate is about 36 to about 52 amino acids in length. In some embodiments, the peptide of the peptide-lipid conjugate is about 40 to about 52 amino acids in length. In some embodiments, the peptide of the peptide-lipid conjugate is about 44 to about 52 amino acids in length. In some embodiments, the peptide of the peptide-lipid conjugate is about 48 to about 52 amino acids in length.

In some embodiments, the peptide of the peptide-lipid conjugate is about 4 to about 48 amino acids in length. In some embodiments, the peptide of the peptide-lipid conjugate is about 4 to about 44 amino acids in length. In some embodiments, the peptide of the peptide-lipid conjugate is about 4 to about 40 amino acids in length. In some embodiments, the peptide of the peptide-lipid conjugate is about 4 to about 36 amino acids in length. In some embodiments, the peptide of the peptide-lipid conjugate is about 4 to about 32 amino acids in length. In some embodiments, the peptide of the peptide-lipid conjugate is about 4 to about 28 amino acids in length. In some embodiments, the peptide of the peptide-lipid conjugate is about 4 to about 24 amino acids in length. In some embodiments, the peptide of the peptide-lipid conjugate is about 4 to about 20 amino acids in length. In some embodiments, the peptide of the peptide-lipid conjugate is about 4 to about 16 amino acids in length. In some embodiments, the peptide of the peptide-lipid conjugate is about 4 to about 12 amino acids in length. In some embodiments, the peptide of the peptide-lipid conjugate is about 4 to about 8 amino acids in length. In some embodiments, the peptide of the peptide-lipid conjugate is about 4 amino acids, 8 amino acids, 12 amino acids, 16 amino acids, 20 amino acids, 24 amino acids, 28 amino acids, 32 amino acids, 36 amino acids, 40 amino acids, 44 amino acids, 48 amino acids or 52 amino acids in length. In some embodiments, the peptide of the peptide-lipid conjugate is about 12 amino acids in length. In some embodiments, the peptide of the peptide-lipid conjugate is 12 amino acids in length. In some embodiments, the peptide of the peptide-lipid conjugate is about 16 amino acids in length. In some embodiments, the peptide of the peptide-lipid conjugate is 16 amino acids in length. In some embodiments, the peptide of the peptide-lipid conjugate is about 20 amino acids in length. In some embodiments, the peptide of the peptide-lipid conjugate is 20 amino acids in length. In some embodiments, the peptide of the peptide-lipid conjugate is about 24 amino acids in length. In some embodiments, the peptide of the peptide-lipid conjugate is 24 amino acids in length. In some embodiments, the peptide of the peptide-lipid conjugate is about 28 amino acids in length. In some embodiments, the peptide of the peptide-lipid conjugate is 28 amino acids in length. In some embodiments, the peptide of the peptide-lipid conjugate is about 32 amino acids in length. In some embodiments, the peptide of the peptide-lipid conjugate is 32 amino acids in length. In some embodiments, the peptide of the peptide-lipid conjugate is about 36 amino acids in length. In some embodiments, the peptide of the peptide-lipid conjugate is 36 amino acids in length. In some embodiments, the peptide of the peptide-lipid conjugate is about 40 amino acids in length. In some embodiments, the peptide of the peptide-lipid conjugate is 40 amino acids in length.

In some embodiments, the peptide of the peptide-lipid conjugate is about 8 to about 50 amino acids in length. In some embodiments, the peptide of the peptide-lipid conjugate is about 8 to about 44 amino acids in length. In some embodiments, the peptide of the peptide-lipid conjugate is about 8 to about 40 amino acids in length. In some embodiments, the peptide of the peptide-lipid conjugate is about 8 to about 36 amino acids in length. In some embodiments, the peptide of the peptide-lipid conjugate is about 8 to about 32 amino acids in length. In some embodiments, the peptide of the peptide-lipid conjugate is about 8 to about 28 amino acids in length. In some embodiments, the peptide of the peptide-lipid conjugate is about 8 to about 24 amino acids in length. In some embodiments, the peptide of the peptide-lipid conjugate is about 8 to about 20 amino acids in length. In some embodiments, the peptide of the peptide-lipid conjugate is about 4 amino acids, 8 amino acids, 12 amino acids, 16 amino acids, 20 amino acids, 24 amino acids, 28 amino acids, 32 amino acids, 36 amino acids, 40 amino acids, 44 amino acids, 48 amino acids, or 52 amino acids in length.

In some embodiments, at least about 14% of the amino acids in the peptide of the peptide-lipid conjugate are proline. In some embodiments, about 14% to about 60% of the amino acids in the peptide are proline. In some embodiments, about 20% to about 60% of the amino acids in the peptide are proline. In some embodiments, about 25% to about 60% of the amino acids in the peptide are proline.

In some embodiments, about 14% to about 60% of the amino acids in the peptide are proline. In some embodiments, about 20% to about 60% of the amino acids in the peptide are proline. In some embodiments, about 25% to about 60% of the amino acids in the peptide are proline. In some embodiments, about 30% to about 60% of the amino acids in the peptide are proline. In some embodiments, about 35% to about 60% of the amino acids in the peptide are proline. In some embodiments, about 40% to about 60% of the amino acids in the peptide are proline. In some embodiments, about 45% to about 60% of the amino acids in the peptide are proline. In some embodiments, about 50% to about 60% of the amino acids in the peptide are proline. In some embodiments, about 55% to about 60% of the amino acids in the peptide are proline.

In some embodiments, about 14% to about 60% of the amino acids in the peptide are proline. In some embodiments, about 14% to about 55% of the amino acids in the peptide are proline. In some embodiments, about 14% to about 50% of the amino acids in the peptide are proline. In some embodiments, about 14% to about 45% of the amino acids in the peptide are proline. In some embodiments, about 14% to about 40% of the amino acids in the peptide are proline. In some embodiments, about 14% to about 35% of the amino acids in the peptide are proline. In some embodiments, about 14% to about 30% of the amino acids in the peptide are proline. In some embodiments, about 14% to about 25% of the amino acids in the peptide are proline. In some embodiments, about 14% to about 20% of the amino acids in the peptide are proline. In some embodiments, about 14% to about 15% of the amino acids in the peptide are proline. In some embodiments, about 14%, 20%, 25%, 30%, 35%, 40%, 45%, 50%, 55%, or 60% of the amino acids in the peptide are proline.

In some embodiments, about 28% to about 85% of the amino acids in the peptide of the peptide-lipid conjugate have a hydrophilic side chain. In some embodiments, about 35% to about 85% of the amino acids in the peptide of the peptide-lipid conjugate have a hydrophilic side chain.

In some embodiments, about 40% to about 85% of the amino acids in the peptide of the peptide-lipid conjugate have a hydrophilic side chain. In some embodiments, about 45% to about 85% of the amino acids in the peptide of the peptide-lipid conjugate have a hydrophilic side chain. In some embodiments, about 50% to about 85% of the amino acids in the peptide of the peptide-lipid conjugate have a hydrophilic side chain. In some embodiments, about 55% to about 85% of the amino acids in the peptide of the peptide-lipid conjugate have a hydrophilic side chain. In some embodiments, about 60% to about 85% of the amino acids in the peptide of the peptide-lipid conjugate have a hydrophilic side chain. In some embodiments, about 65% to about 85% of the amino acids in the peptide of the peptide-lipid conjugate have a hydrophilic side chain. In some embodiments, about 70% to about 85% of the amino acids in the peptide of the peptide-lipid conjugate have a hydrophilic side chain. In some embodiments, about 75% to about 85% of the amino acids in the peptide of the peptide-lipid conjugate have a hydrophilic side chain. In some embodiments, about 80% to about 85% of the amino acids in the peptide of the peptide-lipid conjugate have a hydrophilic side chain.

In some embodiments, about 28% to about 80% of the amino acids in the peptide of the peptide-lipid conjugate have a hydrophilic side chain. In some embodiments, about 75% to about 85% of the amino acids in the peptide of the peptide-lipid conjugate have a hydrophilic side chain. In some embodiments, about 28% to about 70% of the amino acids in the peptide of the peptide-lipid conjugate have a hydrophilic side chain. In some embodiments, about 28% to about 65% of the amino acids in the peptide of the peptide-lipid conjugate have a hydrophilic side chain. In some embodiments, about 28% to about 60% of the amino acids in the peptide of the peptide-lipid conjugate have a hydrophilic side chain. In some embodiments, about 28% to about 55% of the amino acids in the peptide of the peptide-lipid conjugate have a hydrophilic side chain. In some embodiments, about 28% to about 50% of the amino acids in the peptide of the peptide-lipid conjugate have a hydrophilic side chain. In some embodiments, about 28% to about 45% of the amino acids in the peptide of the peptide-lipid conjugate have a hydrophilic side chain. In some embodiments, about 28% to about 40% of the amino acids in the peptide of the peptide-lipid conjugate have a hydrophilic side chain. In some embodiments, about 28% to about 35% of the amino acids in the peptide of the peptide-lipid conjugate have a hydrophilic side chain.

In some embodiments, about 28% to about 80% of the amino acids in the peptide of the peptide-lipid conjugate have a hydrophilic side chain. In some embodiments, about 35% to about 85% of the amino acids in the peptide have a hydrophilic side chain. In some embodiments, about 35% to about 80% of the amino acids in the peptide have a hydrophilic side chain. In some embodiments, about 40% to about 75% of the amino acids in the peptide have a hydrophilic side chain. In some embodiments, about 45% to about 85% of the amino acids in the peptide have a hydrophilic side chain. In some embodiments, about 50% to about 75% of the amino acids in the peptide have a hydrophilic side chain. In some embodiments, about 28%, 35%, 40%, 45%, 50%, 55%, 60%, 65%, 70%, 75%, or 80% of the amino acids in the peptide of the peptide-lipid conjugate have a hydrophilic side chain.

In some embodiments, the amino acids having a hydrophilic side chain are independently selected from glutamine, glutamic acid, asparagine, aspartic acid, serine, O—C₁₋₆ alkyl serine, threonine, and O—C₁₋₆alkyl threonine. In some embodiments, the amino acids having a hydrophilic side chain comprise glutamine, glutamic acid, asparagine, aspartic acid, serine, O—C₁₋₆ alkyl serine, threonine, and O—C₁₋₆alkyl threonine. In some embodiments, the amino acids having a hydrophilic side chain comprise glutamine. In some embodiments, the amino acids having a hydrophilic side chain comprise glutamic acid. In some embodiments, the amino acids having a hydrophilic side chain comprise asparagine. In some embodiments, the amino acids having a hydrophilic side chain comprise aspartic acid. In some embodiments, the amino acids having a hydrophilic side chain comprise serine. In some embodiments, the amino acids having a hydrophilic side chain comprise O—C₁₋₆ alkyl serine. In some embodiments, the amino acids having a hydrophilic side chain comprise threonine. In some embodiments, the amino acids having a hydrophilic side chain comprise O—C₁₋₆ alkyl threonine.

In some embodiments, the O—C₁₋₆ alkyl serine is O—C₁ alkyl serine. In some embodiments, the O—C₁₋₆ alkyl serine is O—C₂ alkyl serine. In some embodiments, the O—C₁₋₆ alkyl serine is O—C₃ alkyl serine. In some embodiments, the O—C₁₋₆ alkyl serine is O—C₄ alkyl serine. In some embodiments, the O—C₁₋₆alkyl serine is O—C₅ alkyl serine. In some embodiments, the O—C₁₋₆ alkyl serine is O—C₆ alkyl serine.

In some embodiments, the O—C₁₋₆ alkyl threonine is O—C₁ alkyl threonine. In some embodiments, the O—C₁₋₆ alkyl threonine is O—C₂ alkyl threonine. In some embodiments, the O—C₁₋₆ alkyl threonine is O—C₃ alkyl threonine. In some embodiments, the O—C₁₋₆ alkyl threonine is O—C₄ alkyl threonine. In some embodiments, the O—C₁₋₆ alkyl threonine is O—C₅ alkyl threonine.

In some embodiments, the O—C₁₋₆ alkyl threonine is O—C₆ alkyl threonine.

In some embodiments, less than about 43% of the amino acids in the peptide of the peptide-lipid conjugate are glycine. In some embodiments, less than about 30% of the amino acids in the peptide are glycine. In some embodiments, less than about 20% of the amino acids in the peptide are glycine. In some embodiments, less than about 10% of the amino acids in the peptide are glycine. In some embodiments, less than about 5% of the amino acids in the peptide are glycine. In some embodiments, less than about 4% of the amino acids in the peptide are glycine. In some embodiments, less than about 3% of the amino acids in the peptide are glycine. In some embodiments, less than about 2% of the amino acids in the peptide are glycine. In some embodiments, the peptide does not have glycine.

In some embodiments, the lipid composition is selected from a lipoplex, a liposome, a lipid nanoparticle, a polymer-based carrier, an exosome, a lamellar body, a micelle, and an emulsion. In some embodiments, the lipid composition is a lipoplex. In some embodiments, the lipid composition is a liposome. In some embodiments, the lipid composition is a polymer-based carrier. In some embodiments, the lipid composition is an exosome. In some embodiments, the lipid composition is a lamellar body. In some embodiments, the lipid composition is a micelle. In some embodiments, the lipid composition is an emulsion. In some embodiments, the lipid composition is a lipid nanoparticle.

In some embodiments, the liposome is selected from a cationic liposome, a nanoliposome, a proteoliposome, a unilamellar liposome, a multilamellar liposome, a ceramide-containing nanoliposome, and a multivesicular liposome. In some embodiments, the liposome is a cationic liposome, a nanoliposome, a proteoliposome, a unilamellar liposome, a multilamellar liposome, a ceramide-containing nanoliposome, and a multivesicular liposome. In some embodiments, the liposome is a cationic liposome. In some embodiments, the liposome is a nanoliposome. In some embodiments, the liposome is a proteoliposome. In some embodiments, the liposome is a unilamellar liposome. In some embodiments, the liposome is a multilamellar liposome. In some embodiments, the liposome is a ceramide-containing nanoliposome. In some embodiments, the liposome is a multivesicular liposome.

In some embodiments, the lipid nanoparticle has a size of less than about 200 nm.

In some embodiments, the lipid composition comprises one or more cationic lipids, one or more helper lipids, and a sterol.

In some embodiments, the peptide-lipid conjugate comprises about 0.1 mol % to about 10 mol % of all lipids in the lipid composition. In some embodiments, the peptide-lipid conjugate comprises about 0.4 mol % to about 10 mol % of all lipids in the lipid composition. In some embodiments, the peptide-lipid conjugate comprises about 0.7 mol % to about 10 mol % of all lipids in the lipid composition. In some embodiments, the peptide-lipid conjugate comprises about 1 mol % to about 10 mol % of all lipids in the lipid composition. In some embodiments, the peptide-lipid conjugate comprises about 1.3 mol % to about 10 mol % of all lipids in the lipid composition. In some embodiments, the peptide-lipid conjugate comprises about 1.6 mol % to about 10 mol % of all lipids in the lipid composition. In some embodiments, the peptide-lipid conjugate comprises about 1.9 mol % to about 10 mol % of all lipids in the lipid composition. In some embodiments, the peptide-lipid conjugate comprises about 2.2 mol % to about 10 mol % of all lipids in the lipid composition. In some embodiments, the peptide-lipid conjugate comprises about 2.5 mol % to about 10 mol % of all lipids in the lipid composition. In some embodiments, the peptide-lipid conjugate comprises about 2.8 mol % to about 10 mol % of all lipids in the lipid composition. In some embodiments, the peptide-lipid conjugate comprises about 3.1 mol % to about 10 mol % of all lipids in the lipid composition. In some embodiments, the peptide-lipid conjugate comprises about 3.4 mol % to about 10 mol % of all lipids in the lipid composition. In some embodiments, the peptide-lipid conjugate comprises about 3.7 mol % to about 10 mol % of all lipids in the lipid composition. In some embodiments, the peptide-lipid conjugate comprises about 4 mol % to about 10 mol % of all lipids in the lipid composition. In some embodiments, the peptide-lipid conjugate comprises about 4.3 mol % to about 10 mol % of all lipids in the lipid composition. In some embodiments, the peptide-lipid conjugate comprises about 4.6 mol % to about 10 mol % of all lipids in the lipid composition. In some embodiments, the peptide-lipid conjugate comprises about 4.9 mol % to about 10 mol % of all lipids in the lipid composition. In some embodiments, the peptide-lipid conjugate comprises about 5.2 mol % to about 10 mol % of all lipids in the lipid composition. In some embodiments, the peptide-lipid conjugate comprises about 5.5 mol % to about 10 mol % of all lipids in the lipid composition. In some embodiments, the peptide-lipid conjugate comprises about 5.8 mol % to about 10 mol % of all lipids in the lipid composition. In some embodiments, the peptide-lipid conjugate comprises about 6.1 mol % to about 10 mol % of all lipids in the lipid composition. In some embodiments, the peptide-lipid conjugate comprises about 6.4 mol % to about 10 mol % of all lipids in the lipid composition. In some embodiments, the peptide-lipid conjugate comprises about 6.7 mol % to about 10 mol % of all lipids in the lipid composition. In some embodiments, the peptide-lipid conjugate comprises about 7 mol % to about 10 mol % of all lipids in the lipid composition. In some embodiments, the peptide-lipid conjugate comprises about 7.3 mol % to about 10 mol % of all lipids in the lipid composition. In some embodiments, the peptide-lipid conjugate comprises about 7.6 mol % to about 10 mol % of all lipids in the lipid composition. In some embodiments, the peptide-lipid conjugate comprises about 7.9 mol % to about 10 mol % of all lipids in the lipid composition. In some embodiments, the peptide-lipid conjugate comprises about 8.2 mol % to about 10 mol % of all lipids in the lipid composition. In some embodiments, the peptide-lipid conjugate comprises about 8.5 mol % to about 10 mol % of all lipids in the lipid composition. In some embodiments, the peptide-lipid conjugate comprises about 8.8 mol % to about 10 mol % of all lipids in the lipid composition. In some embodiments, the peptide-lipid conjugate comprises about 9.1 mol % to about 10 mol % of all lipids in the lipid composition. In some embodiments, the peptide-lipid conjugate comprises about 9.4 mol % to about 10 mol % of all lipids in the lipid composition. In some embodiments, the peptide-lipid conjugate comprises about 9.7 mol % to about 10 mol % of all lipids in the lipid composition.

In some embodiments, the peptide-lipid conjugate comprises about 0.1 mol % to about 9.7 mol % of all lipids in the lipid composition. In some embodiments, the peptide-lipid conjugate comprises about 0.1 mol % to about 9.4 mol % of all lipids in the lipid composition. In some embodiments, the peptide-lipid conjugate comprises about 0.1 mol % to about 9.1 mol % of all lipids in the lipid composition. In some embodiments, the peptide-lipid conjugate comprises about 0.1 mol % to about 8.8 mol % of all lipids in the lipid composition. In some embodiments, the peptide-lipid conjugate comprises about 0.1 mol % to about 8.5 mol % of all lipids in the lipid composition. In some embodiments, the peptide-lipid conjugate comprises about 0.1 mol % to about 8.2 mol % of all lipids in the lipid composition. In some embodiments, the peptide-lipid conjugate comprises about 0.1 mol % to about 7.9 mol % of all lipids in the lipid composition. In some embodiments, the peptide-lipid conjugate comprises about 0.1 mol % to about 7.6 mol % of all lipids in the lipid composition. In some embodiments, the peptide-lipid conjugate comprises about 0.1 mol % to about 7.3 mol % of all lipids in the lipid composition. In some embodiments, the peptide-lipid conjugate comprises about 0.1 mol % to about 7 mol % of all lipids in the lipid composition. In some embodiments, the peptide-lipid conjugate comprises about 0.1 mol % to about 6.7 mol % of all lipids in the lipid composition. In some embodiments, the peptide-lipid conjugate comprises about 0.1 mol % to about 6.4 mol % of all lipids in the lipid composition. In some embodiments, the peptide-lipid conjugate comprises about 0.1 mol % to about 6.1 mol % of all lipids in the lipid composition. In some embodiments, the peptide-lipid conjugate comprises about 0.1 mol % to about 5.8 mol % of all lipids in the lipid composition. In some embodiments, the peptide-lipid conjugate comprises about 0.1 mol % to about 5.5 mol % of all lipids in the lipid composition. In some embodiments, the peptide-lipid conjugate comprises about 0.1 mol % to about 5.2 mol % of all lipids in the lipid composition. In some embodiments, the peptide-lipid conjugate comprises about 0.1 mol % to about 4.9 mol % of all lipids in the lipid composition. In some embodiments, the peptide-lipid conjugate comprises about 0.1 mol % to about 4.6 mol % of all lipids in the lipid composition. In some embodiments, the peptide-lipid conjugate comprises about 0.1 mol % to about 4.3 mol % of all lipids in the lipid composition. In some embodiments, the peptide-lipid conjugate comprises about 0.1 mol % to about 4 mol % of all lipids in the lipid composition. In some embodiments, the peptide-lipid conjugate comprises about 0.1 mol % to about 3.7 mol % of all lipids in the lipid composition. In some embodiments, the peptide-lipid conjugate comprises about 0.1 mol % to about 3.4 mol % of all lipids in the lipid composition. In some embodiments, the peptide-lipid conjugate comprises about 0.1 mol % to about 3.1 mol % of all lipids in the lipid composition. In some embodiments, the peptide-lipid conjugate comprises about 0.1 mol % to about 2.8 mol % of all lipids in the lipid composition. In some embodiments, the peptide-lipid conjugate comprises about 0.1 mol % to about 2.5 mol % of all lipids in the lipid composition. In some embodiments, the peptide-lipid conjugate comprises about 0.1 mol % to about 2.2 mol % of all lipids in the lipid composition. In some embodiments, the peptide-lipid conjugate comprises about 0.1 mol % to about 1.9 mol % of all lipids in the lipid composition. In some embodiments, the peptide-lipid conjugate comprises about 0.1 mol % to about 1.6 mol % of all lipids in the lipid composition. In some embodiments, the peptide-lipid conjugate comprises about 0.1 mol % to about 1.3 mol % of all lipids in the lipid composition. In some embodiments, the peptide-lipid conjugate comprises about 0.1 mol % to about 1 mol % of all lipids in the lipid composition. In some embodiments, the peptide-lipid conjugate comprises about 0.1 mol % to about 0.7 mol % of all lipids in the lipid composition. In some embodiments, the peptide-lipid conjugate comprises about 0.1 mol % to about 0.4 mol % of all lipids in the lipid composition. In some embodiments, the peptide-lipid conjugate comprises about 0.1 mol %, 0.4 mol %, 0.7 mol %, 1 mol %, 1.3 mol %, 1.6 mol %, 1.9 mol %, 2.2 mol %, 2.5 mol %, 2.8 mol %, 3.1 mol %, 3.4 mol %, 3.7 mol %, 4 mol %, 4.3 mol %, 4.6 mol %, 4.9 mol %, 5.2 mol %, 5.5 mol %, 5.8 mol %, 6.1 mol %, 6.4 mol %, 6.7 mol %, 7 mol %, 7.3 mol %, 7.6 mol %, 7.9 mol %, 8.2 mol %, 8.5 mol %, 8.8 mol %, 9.1 mol %, 9.4 mol %, 9.7 mol %, or 10 mol % of all lipids in the lipid composition.

In some embodiments, the peptide-lipid conjugate comprises about 0.5 mol % to about 5 mol % of all lipids in the lipid composition. In some embodiments, the peptide-lipid conjugate comprises about 1.0 mol % to about 3 mol % of all lipids in the lipid composition.

In some embodiments, the lipid composition encapsulates the nucleic acid.

In some embodiments, the lipid composition is complexed to the nucleic acid.

In some embodiments, the lipid of the peptide lipid-conjugate is conjugated to the peptide via a linker. In some embodiments, the linker is a bond, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. In embodiments, the linker is a substituted or unsubstituted alkyl (e.g., C₁-C₈, C₁-C₆, or C₁-C₄), substituted or unsubstituted heteroalkyl, substituted or unsubstituted cycloalkyl (e.g., 2 to 8 membered, 2 to 6 membered, or 2 to 4 membered), substituted or unsubstituted heterocycloalkyl (e.g., 3 to 8 membered, 3 to 6 membered, or 5 to 6 membered), substituted or unsubstituted aryl (e.g., C₆-C₁₀ or phenyl), or substituted or unsubstituted heteroaryl (e.g., 5 to 10 membered, 5 to 9 membered, or 5 to 6 membered). In some embodiments, the linker is a substituted or unsubstituted alkyl. In some embodiments, the linker is a substituted or unsubstituted heteroalkyl. In some embodiments, the linker is a substituted or unsubstituted cycloalkyl. In some embodiments, the linker is a substituted or unsubstituted heterocycloalkyl. In some embodiments, the linker is a substituted or unsubstituted aryl. In some embodiments, the linker is a substituted or unsubstituted heteroaryl.

In some embodiments, the linker has a structure comprising a group selected from, —S—, —C(O)O—, amido (—C(O)NH—) or amino (—NR^{N}—) wherein R^{N} is selected from H, C₁₋₆ alkyl, carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl (—NHC(O)CH₂CH₂C(O)NH—), ether, carbonate (—OC(O)O—), succinoyl, phosphate esters (—O—(O)POH—O—), and sulfonate esters. In some embodiments, the linker comprises —S—. In some embodiments, the linker comprises —C(O)O—. In some embodiments, the linker has a structure comprising an amido (—C(O)NH—). In some embodiments, the linker has a structure comprising an amino (—NR^{N}—) wherein R^{N} is selected from H, C₁₋₆ alkyl, carbonyl (—C(O)—), carbamate (—NHC(O)O—), urea (—NHC(O)NH—), disulfide (—S—S—), ether (—O—), succinyl (—(O)CCH₂CH₂C(O)—), succinamidyl (—NHC(O)CH₂CH₂C(O)NH—), ether, carbonate (—OC(O)O—), succinoyl, phosphate esters (—O—(O)POH—O—), and sulfonate esters. In some embodiments, R^{N} is an H. In some embodiments, R^{N} is a C₁₋₆ alkyl. In some embodiments, R^{N} is a C₁ alkyl. In some embodiments, R^{N} is a C₂ alkyl. In some embodiments, R^{N} is a C₃ alkyl. In some embodiments, R^{N} is a C₄ alkyl. In some embodiments, R^{N} is a C₅ alkyl. In some embodiments, R^{N} is a C₆ alkyl. In some embodiments, R^{N} is a carbonyl (—C(O)—). In some embodiments, R^{N} is a carbamate (—NHC(O)O—). In some embodiments, R^{N} is urea (—NHC(O)NH—). In some embodiments, R^{N} is disulfide (—S—S—). In some embodiments, R^{N} is ether (—O—). In some embodiments, R^{N} is succinyl (—(O)CCH₂CH₂C(O)—). In some embodiments, R^{N} is succinamidyl (—NHC(O)CH₂CH₂C(O)NH—). In some embodiments, R^{N} is ether. In some embodiments, R^{N} is carbonate (—OC(O)O—). In some embodiments, R^{N} is succinoyl. In some embodiments, R^{N} is a phosphate ester (—O—(O)POH—O—).

In some embodiments, R^{N} is a sulfonate ester.

In some embodiments, the lipid of the peptide-lipid conjugate is selected from a didecyloxypropyl (C10), a dilauryloxypropyl (C12), a dimyristyloxypropyl (C14), a dipalmityloxypropyl (C16), or a distearyloxypropyl (C18), a 1,2-dimyristyloxypropyl-3-amine (DOMG), a 1,2-dimyristyloxypropylamine (DMG), a 1,2-Dilauroyl-sn-glycero-3-phosphorylethanolamine (DLPE), a dimyristoyl-phosphatidylethanolamine (DMPE), a dipalmitoyl-phosphatidylethanolamine (DPPE), a dipalmitoylphosphatidylcholine (DPPC), a dioleoyl-phosphatidylethanolamine (DOPE), and a distearoyl-phosphatidylethanolamine (DSPE).

In some embodiments, the lipid of the peptide-lipid conjugate is a didecyloxypropyl (C10). In some embodiments, the lipid of the peptide-lipid conjugate is a dilauryloxypropyl (C12). In some embodiments, the lipid of the peptide-lipid conjugate is a dimyristyloxypropyl (C14). In some embodiments, the lipid of the peptide-lipid conjugate is a dipalmityloxypropyl (C16). In some embodiments, the lipid of the peptide-lipid conjugate is a distearyloxypropyl (C18). In some embodiments, the lipid of the peptide-lipid conjugate is a 1,2-dimyristyloxypropyl-3-amine (DOMG). In some embodiments, the lipid of the peptide-lipid conjugate is a 1,2-dimyristyloxypropylamine (DMG). In some embodiments, the lipid of the peptide-lipid conjugate is a 1,2-Dilauroyl-sn-glycero-3-phosphorylethanolamine (DLPE). In some embodiments, the lipid of the peptide-lipid conjugate is a dimyristoyl-phosphatidylethanolamine (DMPE). In some embodiments, the lipid of the peptide-lipid conjugate is a dipalmitoyl-phosphatidylethanolamine (DPPE). In some embodiments, the lipid of the peptide-lipid conjugate is a dipalmitoylphosphatidylcholine (DPPC). In some embodiments, the lipid of the peptide-lipid conjugate is a dioleoyl-phosphatidylethanolamine (DOPE). In some embodiments, the lipid of the peptide-lipid conjugate is a distearoyl-phosphatidylethanolamine (DSPE). In some embodiments, the lipid of the peptide-lipid conjugate is cholesterol or a cholesterol derivative.

In some embodiments, the peptide is conjugated to the lipid of the peptide-lipid conjugate at its C-terminus and the amino group of the N-terminus of the peptide is substituted with one or two C₁₋₆ alkyl groups or amido groups. In some embodiments, the N-terminus of the peptide is substituted with one or two amido groups. In some embodiments, the N-terminus of the peptide is substituted with one or two C₁₋₆ alkyl groups. In some embodiments, the N-terminus of the peptide is substituted with one or two C₁ alkyl groups. In some embodiments, the N-terminus of the peptide is substituted with one or two C₂ alkyl groups. In some embodiments, the N-terminus of the peptide is substituted with one or two C₃ alkyl groups. In some embodiments, the N-terminus of the peptide is substituted with one or two C₄ alkyl groups. In some embodiments, the N-terminus of the peptide is substituted with one or two C₅ alkyl groups. In some embodiments, the N-terminus of the peptide is substituted with one or two C₆ alkyl groups.

In some embodiments, the peptide is conjugated to the lipid of the peptide-lipid conjugate at its N-terminus, and the amino acid at the C-terminus of the peptide is alkylated to form a C₁₋₆ alkyl ester or is amidated. In some embodiments, the amino acid at the C-terminus of the peptide is amidated. In some embodiments, the amino acid at the C-terminus of the peptide is alkylated to form a C₁₋₆ alkyl ester. In some embodiments, the amino acid at the C-terminus of the peptide is alkylated to form a C₁ alkyl ester. In some embodiments, the amino acid at the C-terminus of the peptide is alkylated to form a C₂ alkyl ester. In some embodiments, the amino acid at the C-terminus of the peptide is alkylated to form a C₃ alkyl ester. In some embodiments, the amino acid at the C-terminus of the peptide is alkylated to form a C₄ alkyl ester. In some embodiments, the amino acid at the C-terminus of the peptide is alkylated to form a C₅ alkyl ester. In some embodiments, the amino acid at the C-terminus of the peptide is alkylated to form a C₆ alkyl ester.

In some embodiments, the lipid composition further comprises one or more cationic lipids selected from 5-carboxyspermylglycinedioctadecylamide (DOGS), 2,3-dioleyloxy-N-[2(spermine-carboxamido)ethyl]-N,N-dimethyl-1-propanaminium (DOSPA), 1,2-Dioleoyl-3-Dimethylammonium-Propane (DODAP), 1,2-Dioleoyl-3-Trimethylammonium-Propane (DOTAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), and 2,2-dilinoleyl-4-dimethylaminoethyl-[1,3]-dioxolane or (DLin-K-XTC2-DMA). In some embodiments, the lipid composition further comprises 5-carboxyspermylglycinedioctadecylamide (DOGS). In some embodiments, the lipid composition further comprises 2,3-dioleyloxy-N-[2(spermine-carboxamido)ethyl]-N,N-dimethyl-1-propanaminium (DOSPA). In some embodiments, the lipid composition further comprises 1,2-Dioleoyl-3-Dimethylammonium-Propane (DODAP). In some embodiments, the lipid composition further comprises 1,2-Dioleoyl-3-Trimethylammonium-Propane (DOTAP). In some embodiments, the lipid composition further comprises 1,2-distearyloxy-N,N-dimethyl-3-aminopropane (DSDMA). In some embodiments, the lipid composition further comprises 1,2-dioleyloxy-N,N-dimethyl-3-aminopropane (DODMA). In some embodiments, the lipid composition further comprises 1,2-dilinoleyloxy-N,N-dimethyl-3-aminopropane (DLinDMA). In some embodiments, the lipid composition further comprises N-dioleyl-N,N-dimethylammonium chloride (DODAC). In some embodiments, the lipid composition further comprises N,N-distearyl-N,N-dimethylammonium bromide (DDAB). In some embodiments, the lipid composition further comprises N-(1,2-dimyristyloxyprop-3-yl)-N,N-dimethyl-N-hydroxyethyl ammonium bromide (DMRIE). In some embodiments, the lipid composition further comprises 3-dimethylamino-2-(cholest-5-en-3-beta-oxybutan-4-oxy)-1-(cis,cis-9,12-oc-tadecadienoxy)propane (CLinDMA). In some embodiments, the lipid composition further comprises N,N-dimethyl-3,4-dioleyloxybenzylamine (DMOBA). In some embodiments, the lipid composition further comprises 1,2-N,N′-dioleylcarbamyl-3-dimethylaminopropane (DOcarbDAP). In some embodiments, the lipid composition further comprises 2,3-Dilinoleoyloxy-N,N-dimethylpropylamine (DLinDAP). In some embodiments, the lipid composition further comprises 1,2-N,N′-Dilinoleylcarbamyl-3-dimethylaminopropane (DLincarbDAP). In some embodiments, the lipid composition further comprises 1,2-Dilinoleoylcarbamyl-3-dimethylaminopropane (DLinCDAP). In some embodiments, the lipid composition further comprises 2,2-dilinoleyl-4-dimethylaminomethyl-[1,3]-dioxolane (DLin-K-DMA). In some embodiments, the lipid composition further comprises 2,2-dilinoleyl-4-dimethylaminoethyl-[1,3]-dioxolane. In some embodiments, the lipid composition further comprises 2,2-dilinoleyl-4-dimethylaminoethyl-[1,3]-dioxolane or (DLin-K-XTC2-DMA).

In some embodiments, the lipid composition comprises an ionizable cationic lipid. In some embodiments, the one or more ionizable cationic lipids is selected from

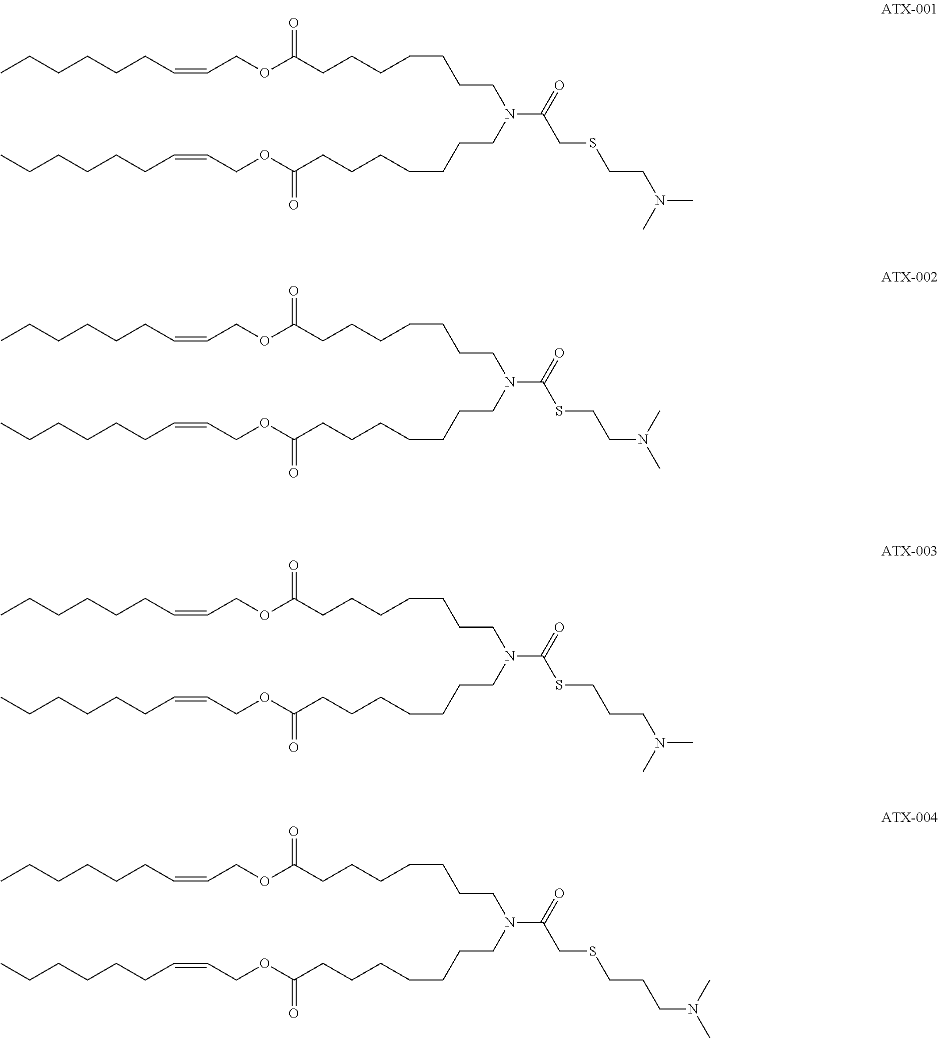

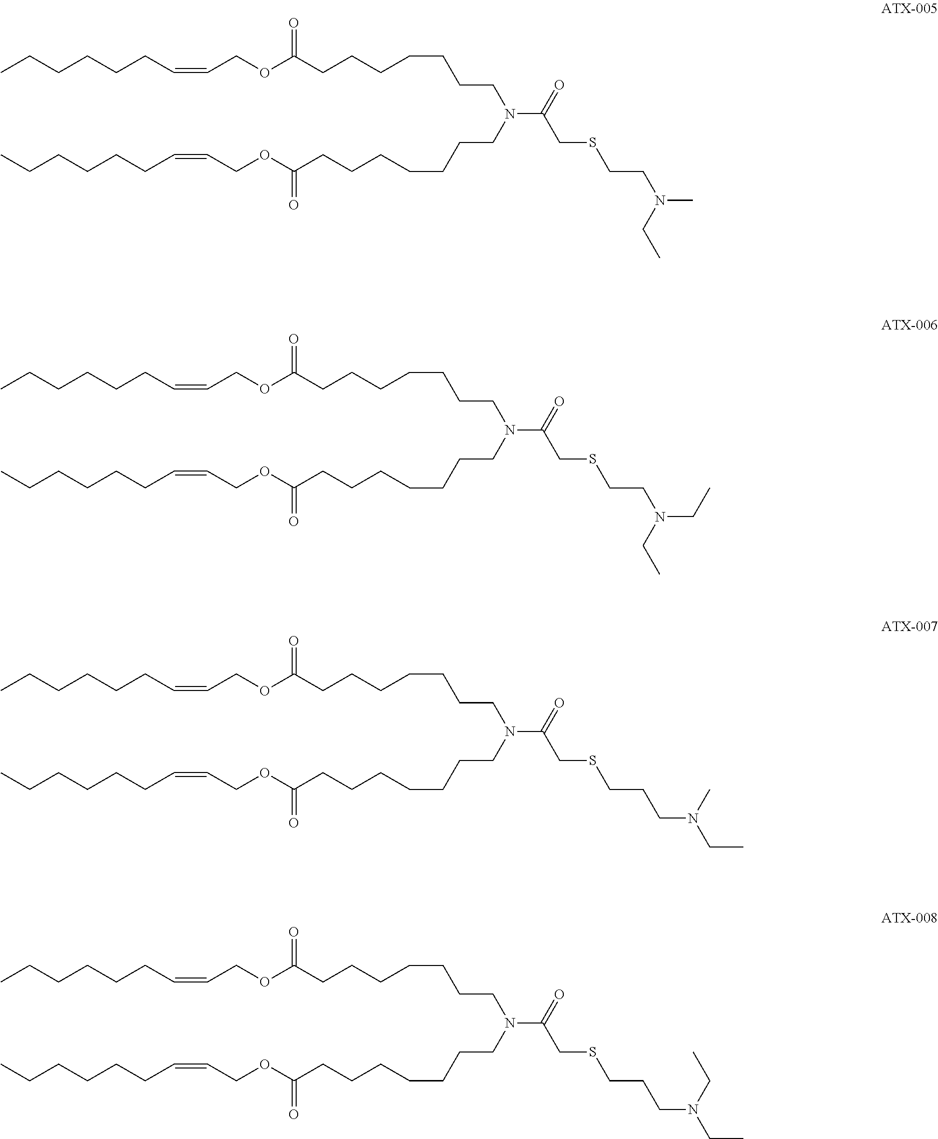

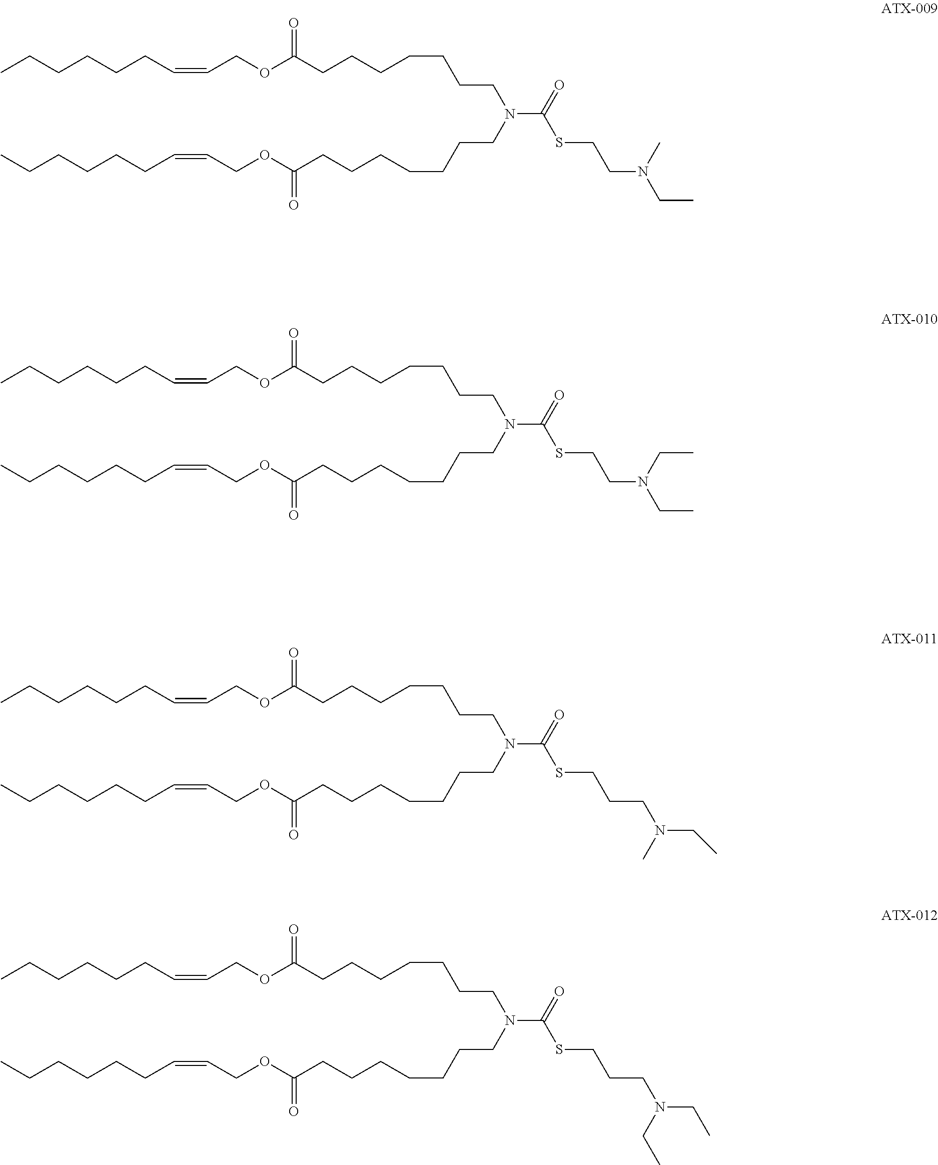

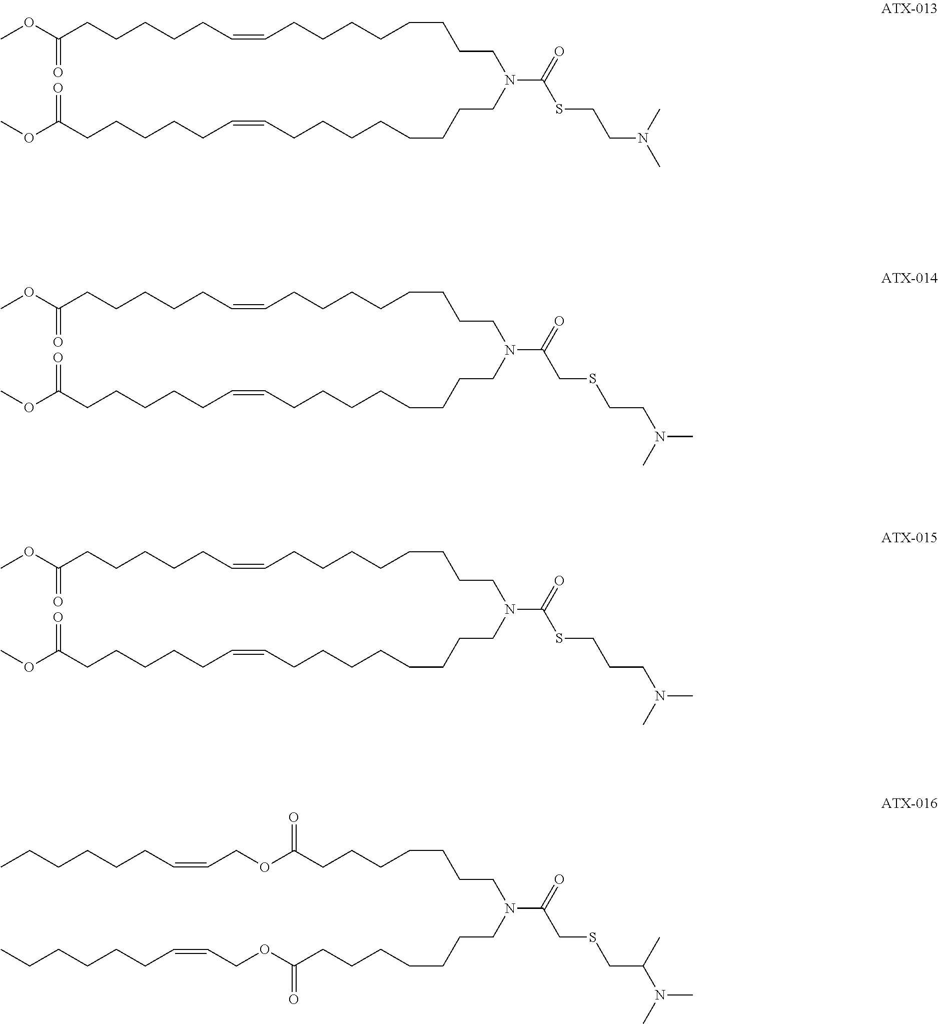

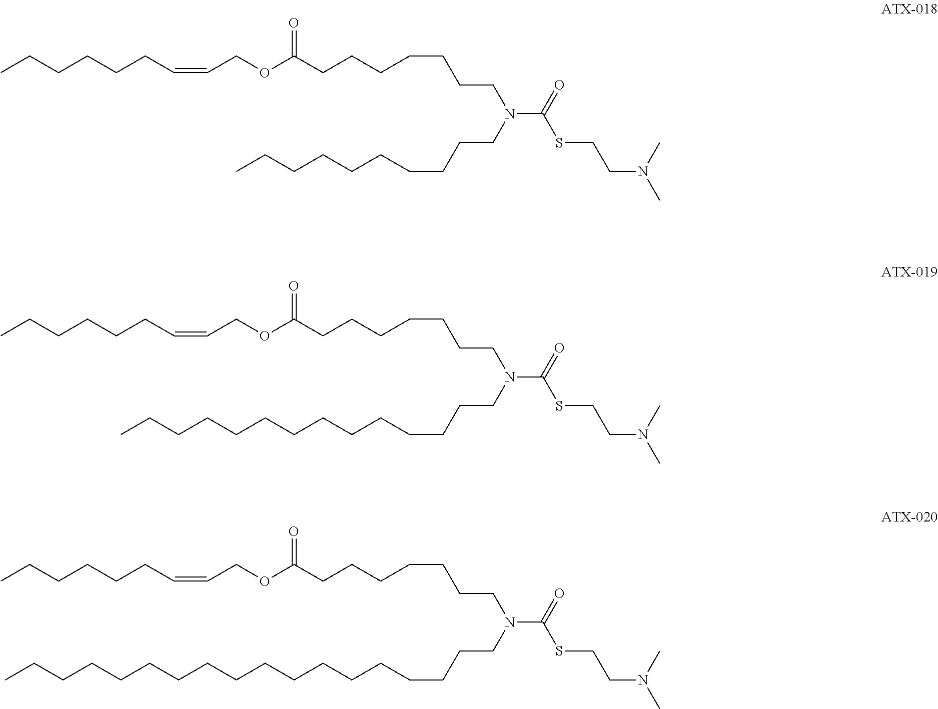

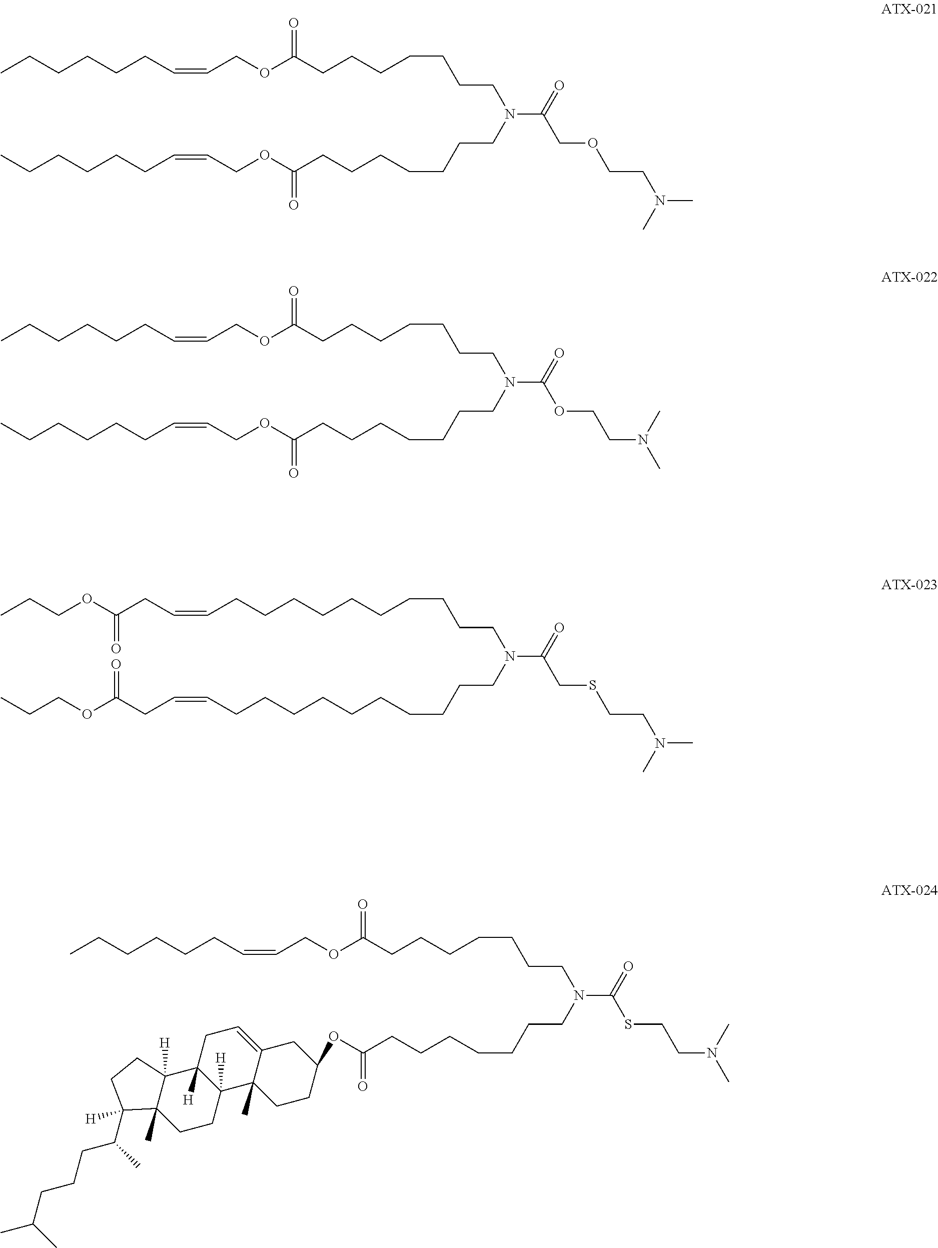

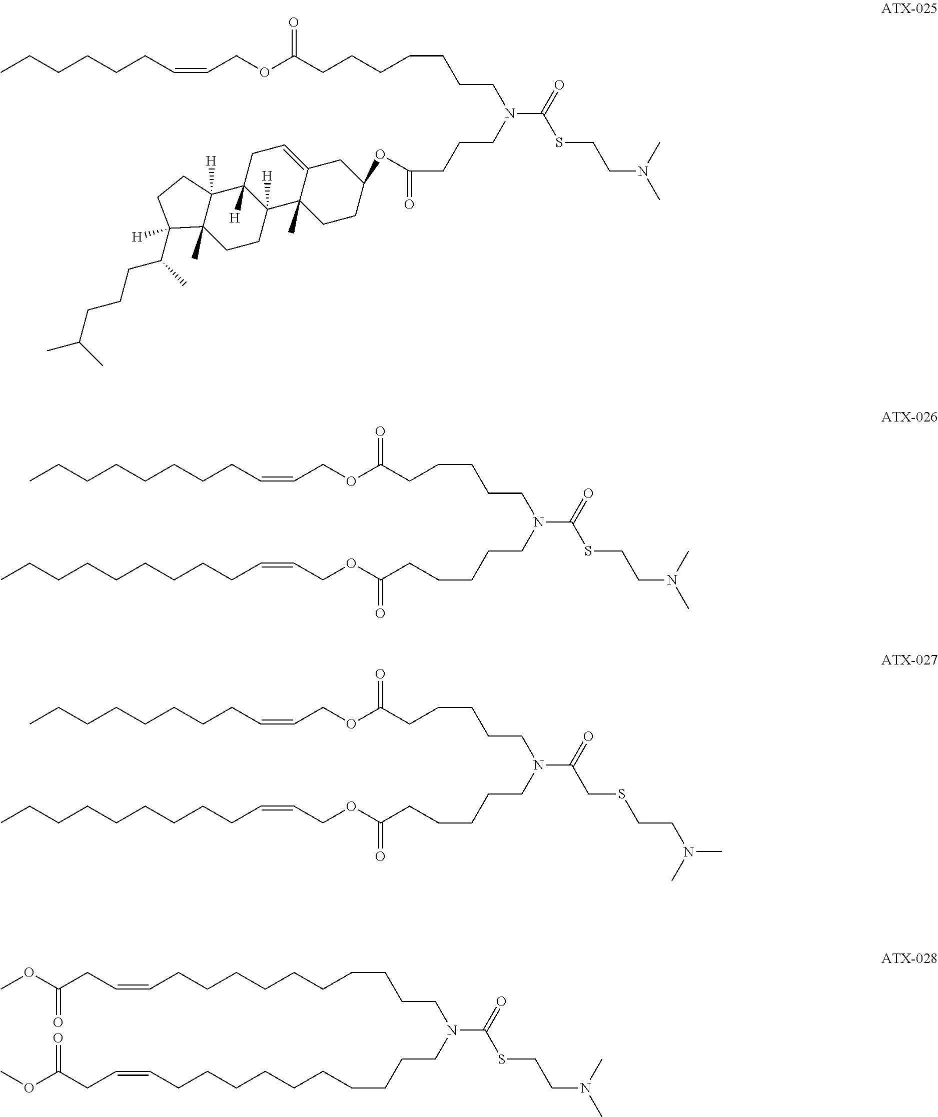

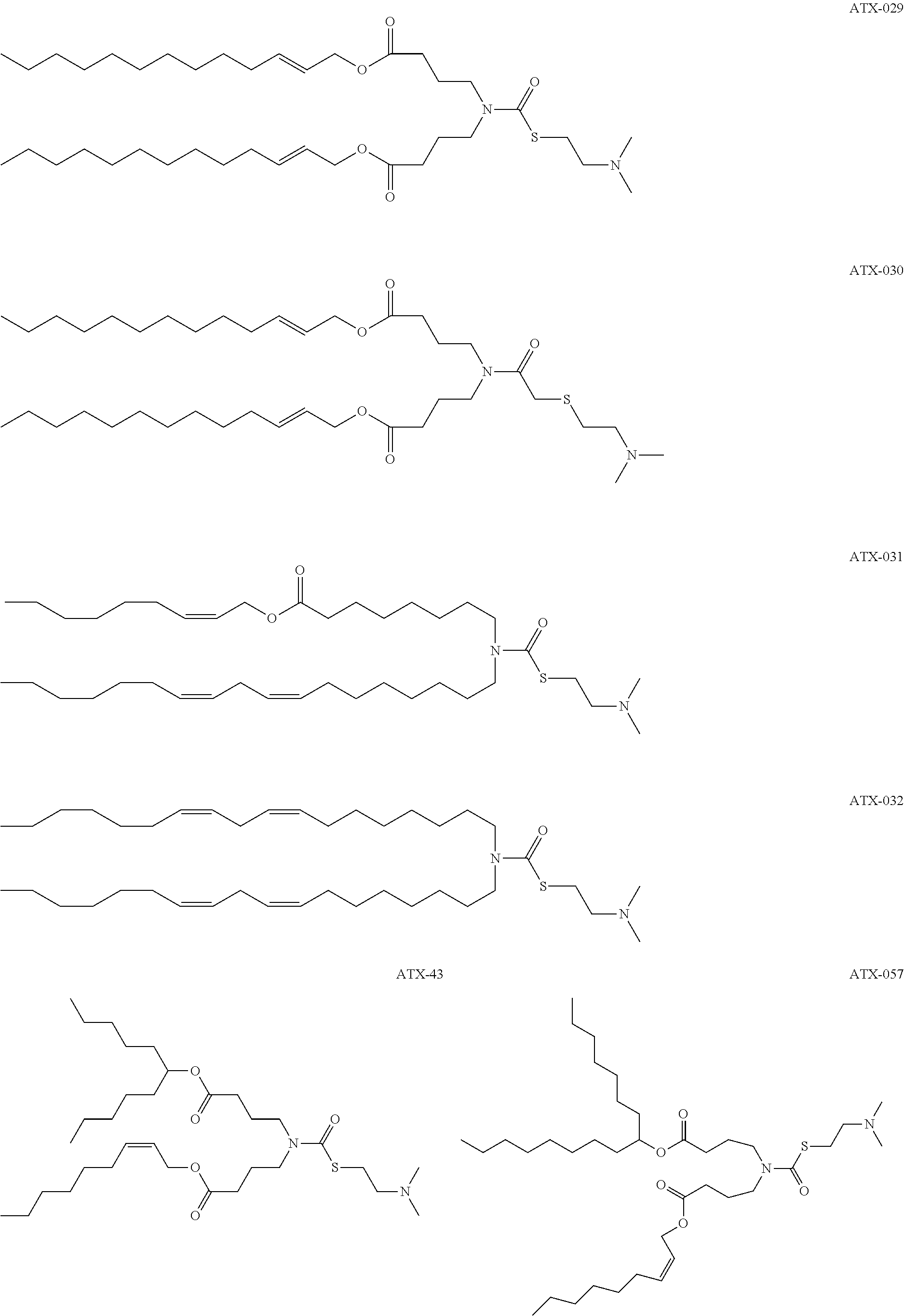

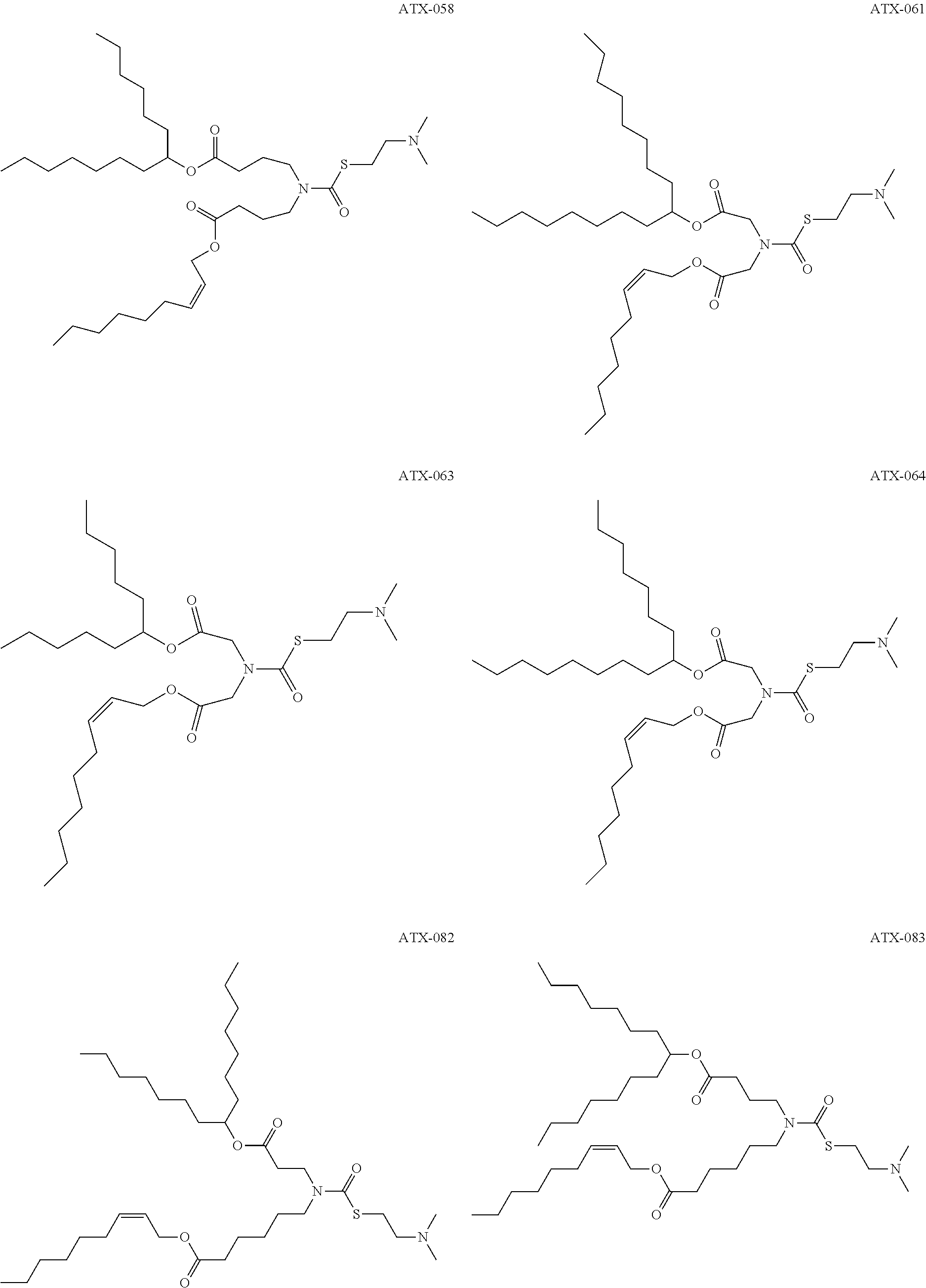

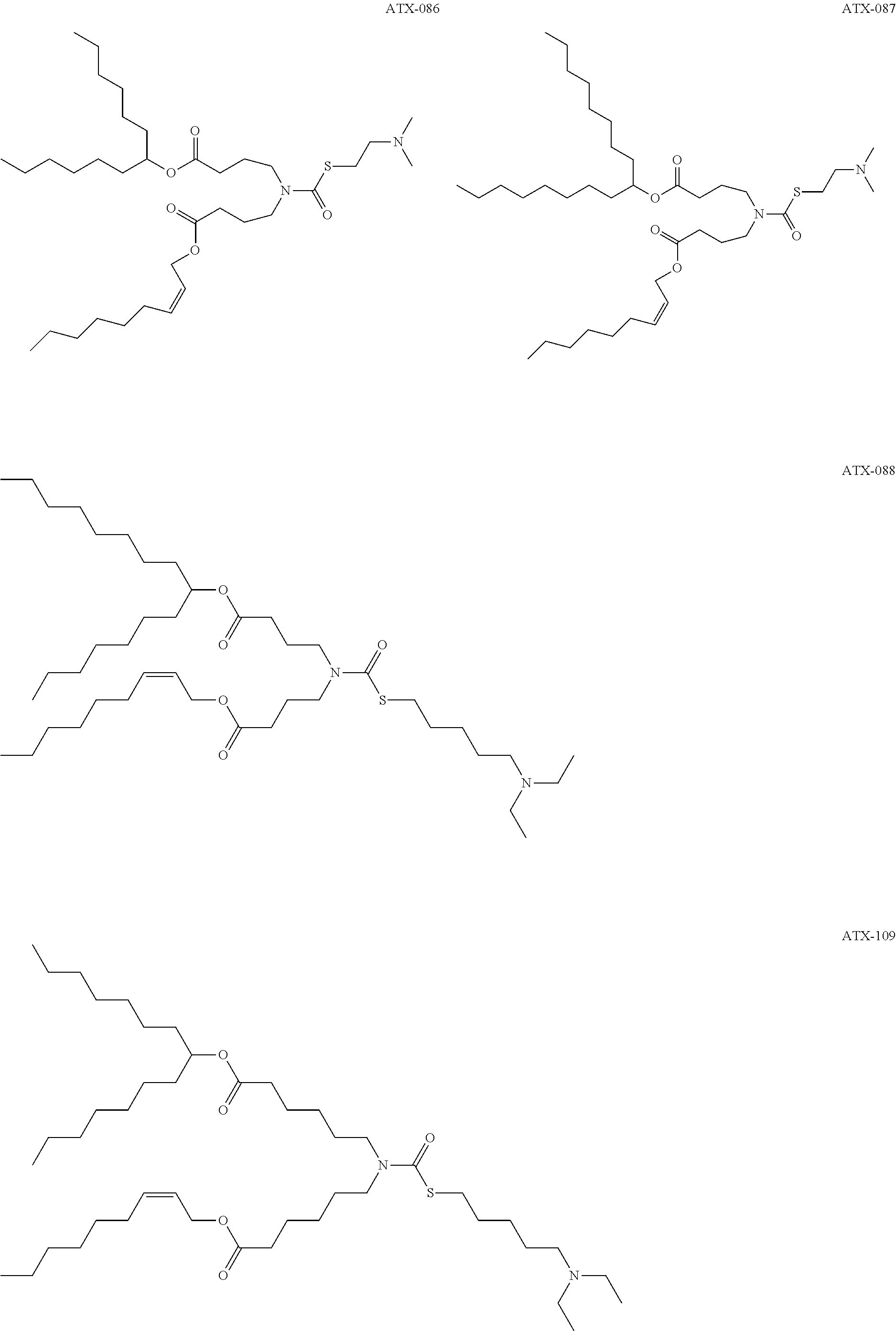

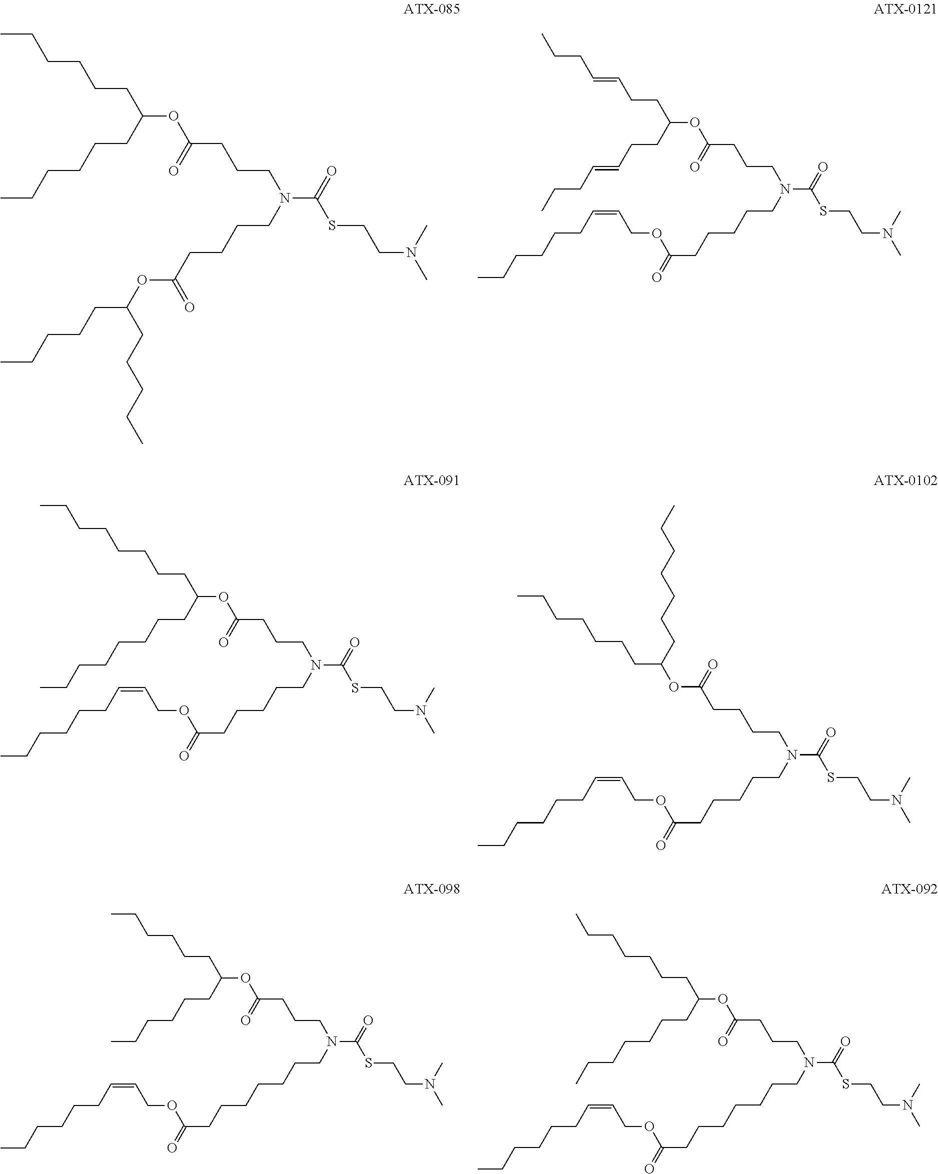 [SMILES: CCCCCCC(CCCCCC)OC(=O)CCCN(CCCCC(=O)OC(CCCCC)CCCCC)C(=O)SCCN(C)C ; CCC/C=C/CCC(CC/C=C/CCC)OC(=O)CCCN(CCCCCC(=O)OC/C=C\CCCCCC)C(=O)SCCN(C)C ; CCCCCC/C=C\COC(=O)CCCCCN(CCCC(=O)OC(CCCCCCCC)CCCCCCCC)C(=O)SCCN(C)C ; CCCCCC/C=C\COC(=O)CCCCCN(CCCCC(=O)OC(CCCCCCC)CCCCCCC)C(=O)SCCN(C)C ; CCCCCC/C=C\COC(=O)CCCCCCCN(CCCC(=O)OC(CCCCCC)CCCCCC)C(=O)SCCN(C)C ; CCCCCC/C=C\COC(=O)CCCCCCCN(CCCC(=O)OC(CCCCCCC)CCCCCCC)C(=O)SCCN(C)C]

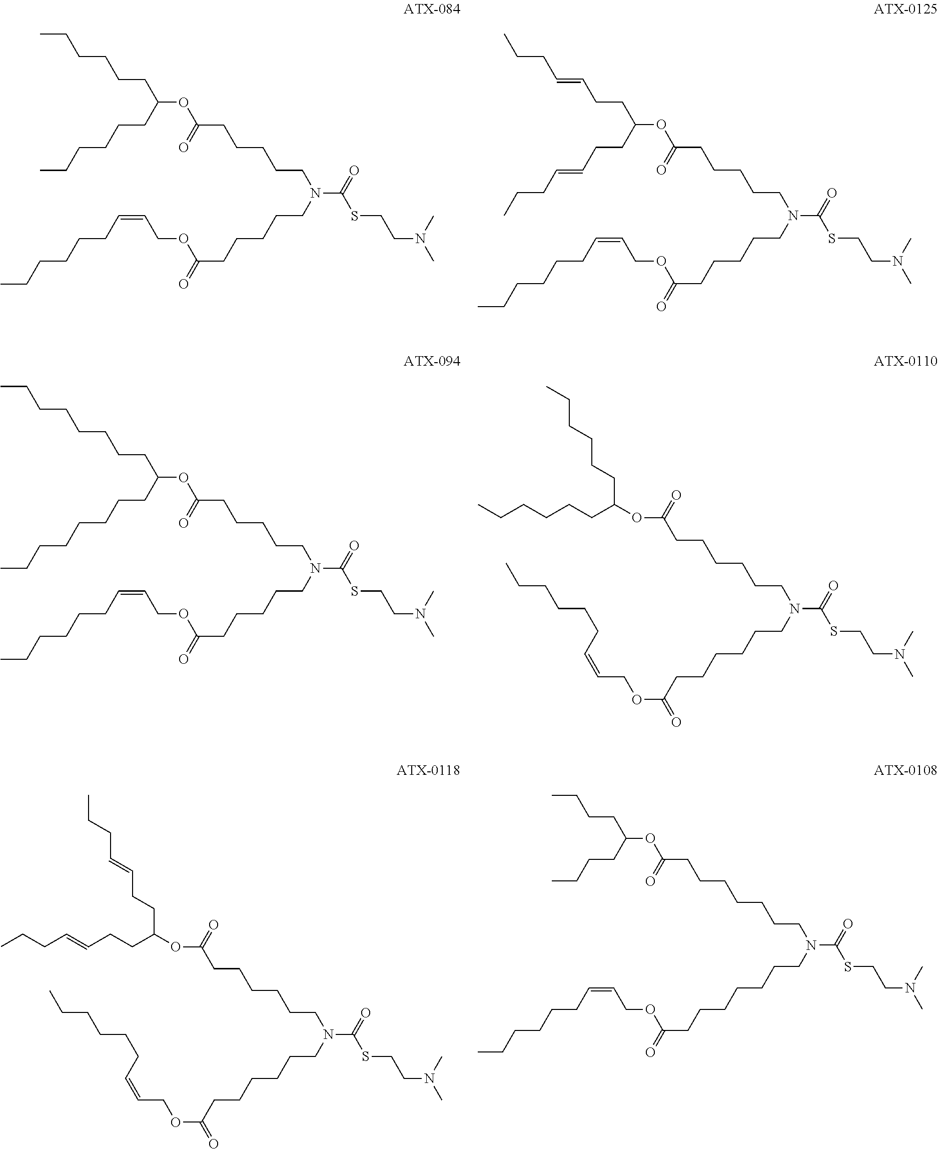

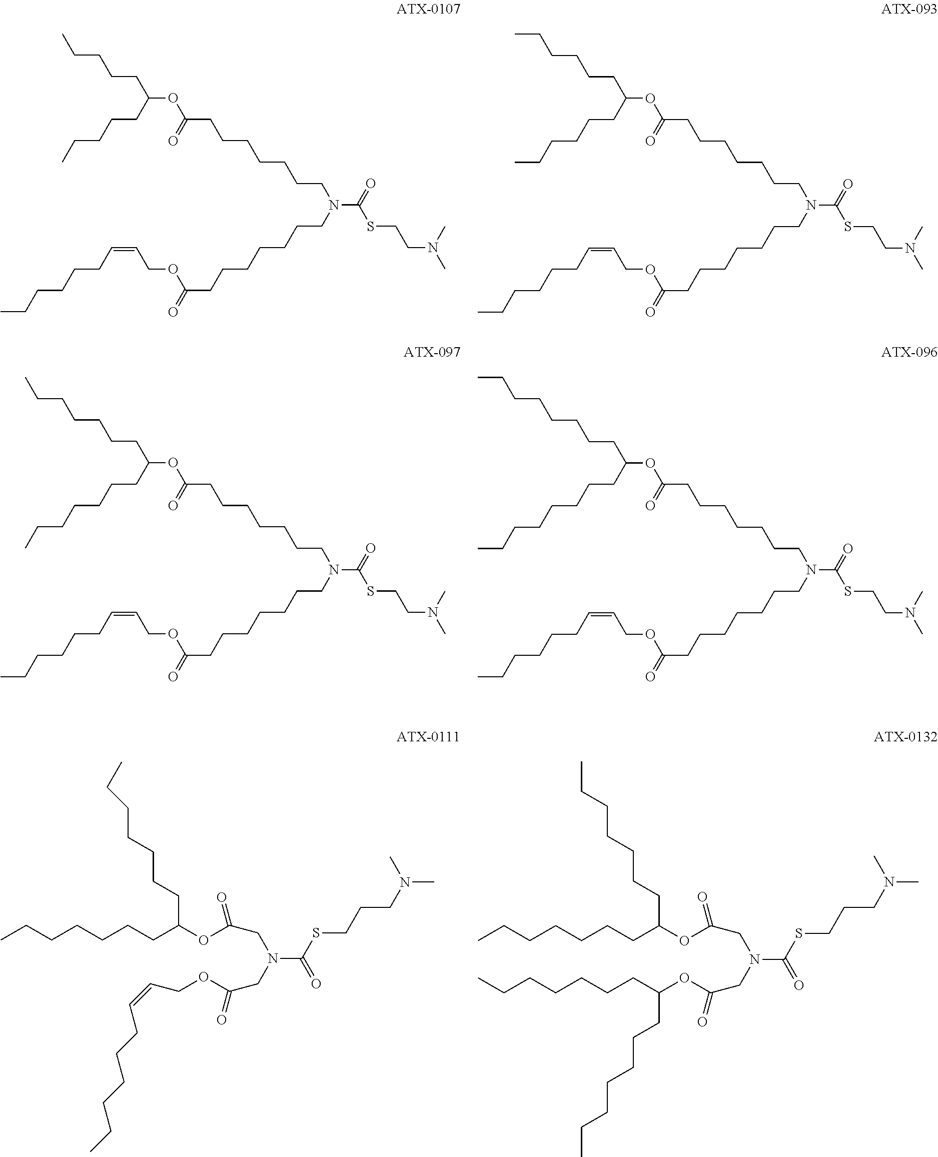 [SMILES: CCCCCC/C=C\COC(=O)CCCCCCCN(CCCCCCCC(=O)OC(CCCCC)CCCCC)C(=O)SCCN(C)C ; CCCCCC/C=C\COC(=O)CCCCCCCN(CCCCCCCC(=O)OC(CCCCCC)CCCCCC)C(=O)SCCN(C)C ; CCCCCC/C=C\COC(=O)CCCCCCCN(CCCCCCCC(=O)OC(CCCCCCC)CCCCCCC)C(=O)SCCN(C)C ; CCCCCC/C=C\COC(=O)CCCCCCCN(CCCCCCCC(=O)OC(CCCCCCCC)CCCCCCCC)C(=O)SCCN(C)C ; CCCCCC/C=C\COC(=O)CN(CC(=O)OC(CCCCCCC)CCCCCCC)C(=O)SCCCN(C)C ; CCCCCCCC(CCCCCCC)OC(=O)CN(CC(=O)OC(CCCCCCC)CCCCCCC)C(=O)SCCCN(C)C]

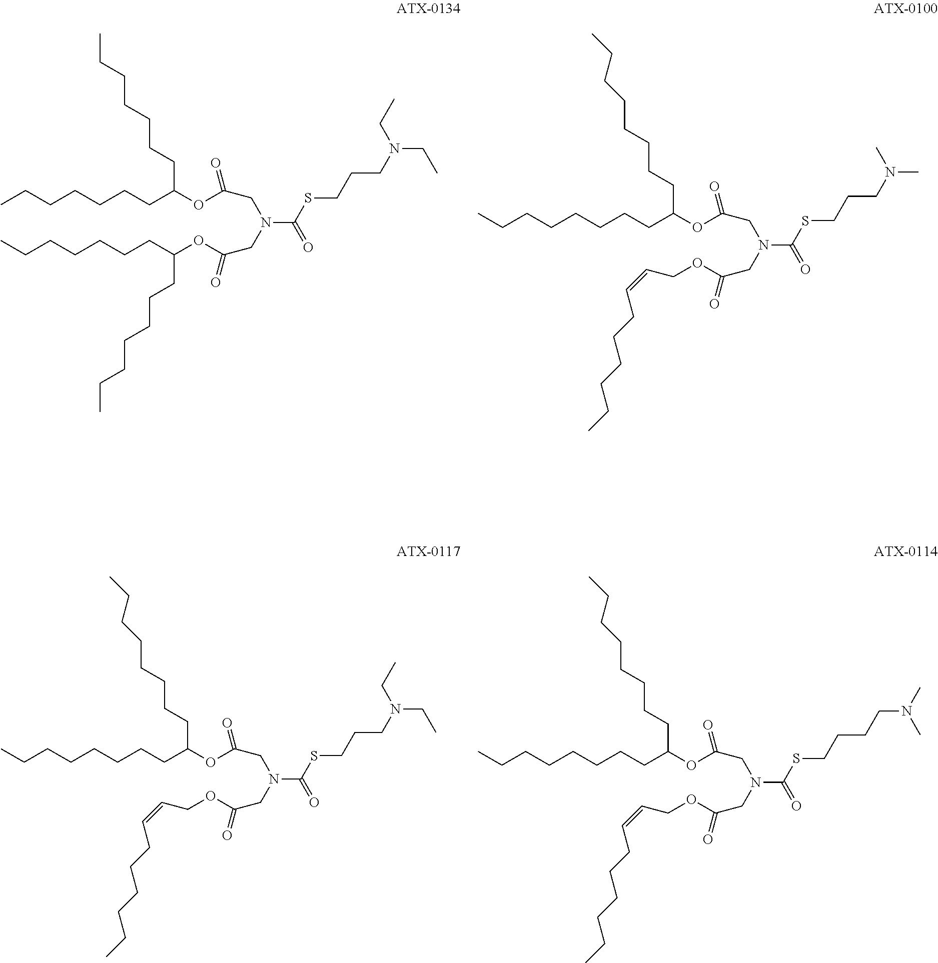 [SMILES: CCCCCCCC(CCCCCCC)OC(=O)CN(CC(=O)OC(CCCCCCC)CCCCCCC)C(=O)SCCCN(CC)CC ; CCCCCC/C=C\COC(=O)CN(CC(=O)OC(CCCCCCCC)CCCCCCCC)C(=O)SCCCN(C)C ; CCCCCC/C=C\COC(=O)CN(CC(=O)OC(CCCCCCCC)CCCCCCCC)C(=O)SCCCN(CC)CC ; CCCCCC/C=C\COC(=O)CN(CC(=O)OC(CCCCCCCC)CCCCCCCC)C(=O)SCCCCN(C)C]

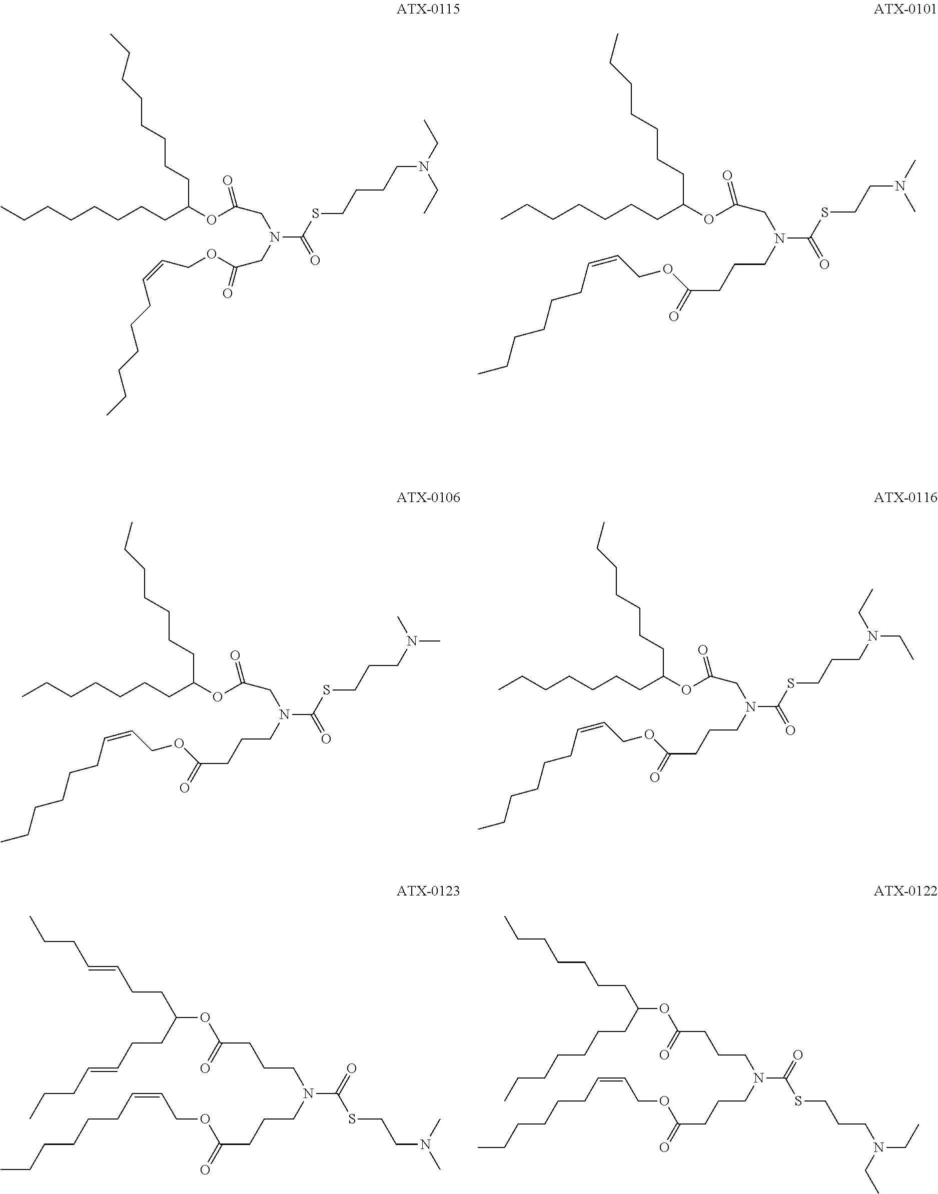 [SMILES: CCCCCC/C=C\COC(=O)CN(CC(=O)OC(CCCCCCCC)CCCCCCCC)C(=O)SCCCCN(CC)CC ; CCCCCC/C=C\COC(=O)CCCN(CC(=O)OC(CCCCCCC)CCCCCCC)C(=O)SCCN(C)C ; CCCCCC/C=C\COC(=O)CCCN(CC(=O)OC(CCCCCCC)CCCCCCC)C(=O)SCCCN(C)C ; CCCCCC/C=C\COC(=O)CCCN(CC(=O)OC(CCCCCCC)CCCCCCC)C(=O)SCCCN(CC)CC ; CCC/C=C/CCC(CC/C=C/CCC)OC(=O)CCCN(CCCC(=O)OC/C=C\CCCCCC)C(=O)SCCN(C)C ; CCCCCC/C=C\COC(=O)CCCN(CCCC(=O)OC(CCCCCCC)CCCCCCC)C(=O)SCCCN(CC)CC]

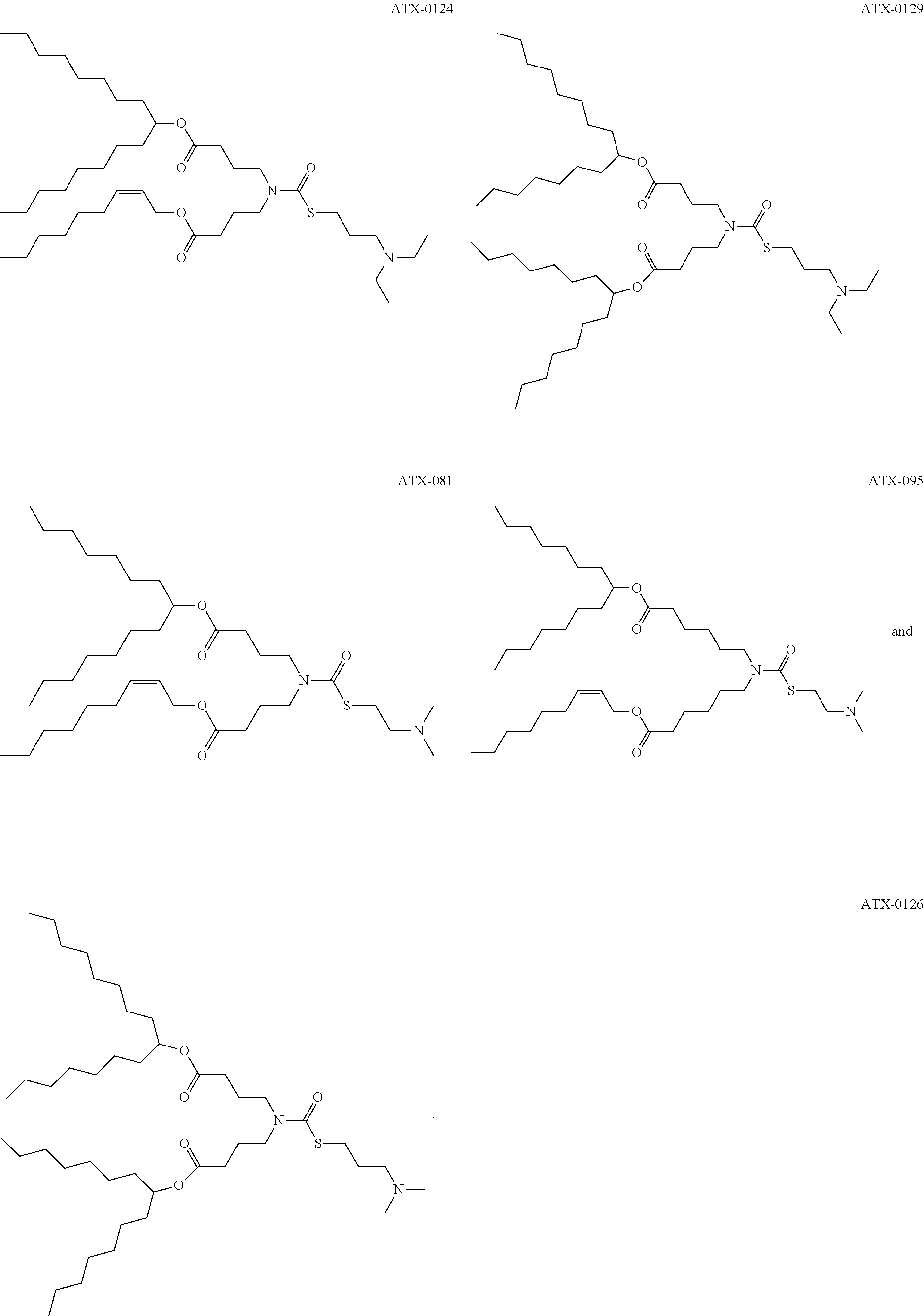 [SMILES: CCCCCC/C=C\COC(=O)CCCN(CCCC(=O)OC(CCCCCCCC)CCCCCCCC)C(=O)SCCCN(CC)CC ; CCCCCCCCC(CCCCCCC)OC(=O)CCCN(CCCC(=O)OC(CCCCCCC)CCCCCCC)C(=O)SCCCN(CC)CC ; CCCCCC/C=C\COC(=O)CCCN(CCCC(=O)OC(CCCCCCC)CCCCCCC)C(=O)SCCN(C)C ; CCCCCC/C=C\COC(=O)CCCCCN(CCCCCC(=O)OC(CCCCCCC)CCCCCCC)C(=O)SCCN(C)C ; CCCCCCCCC(CCCCCCC)OC(=O)CCCN(CCCC(=O)OC(CCCCCCC)CCCCCCC)C(=O)SCCCN(C)C]

In some embodiments, the lipid composition further comprises one or more helper lipids.

In some embodiments, the lipid composition further comprises a sterol. In some embodiments, the sterol is cholesterol.

In one embodiment, a lipid composition is provided comprising a peptide-lipid conjugate provided herein including embodiments thereof, and a nucleic acid, wherein:
i. the peptide of the peptide-lipid conjugate consists of about 4 to about 52 amino acids;
ii. at least about 14% of the amino acids in the peptide of the peptide-lipid conjugate are proline;
iii. about 28% to about 80% of the amino acids in the peptide of the peptide-lipid conjugate have a hydrophilic side chain;
iv. less than about 43% of the amino acids in the peptide of the peptide-lipid conjugate are glycine;
v. the lipid composition comprises one or more cationic lipids, one or more helper lipids, and a sterol; and
vi. the peptide-lipid conjugate comprises about 0.1 mol % to about 10 mol % of all lipids in the lipid composition.

In some embodiments, the nucleic acid is selected from a siRNA, an antisense oligonucleotide, a UNA oligomer, an mRNA, a microRNA, and a DNA. In some embodiments, the nucleic acid is a siRNA. In some embodiments, the nucleic acid is an antisense oligonucleotide. In some embodiments, the nucleic acid is a UNA oligomer. In some embodiments, the nucleic acid is a microRNA. In some embodiments, the nucleic acid is a DNA. In some embodiments, the nucleic acid is an mRNA. In some embodiments, the mRNA is a self-replicating mRNA. In some embodiments, the nucleic acid is a siRNA.

In some embodiments, a method of treating a disease in a subject in need thereof is provided comprising administering a lipid composition of any one of the preceding claims. The disease may be a cancer. The disease may be an autoimmune disease. The disease may be an inflammatory disease. The disease may be an infectious disease. In some embodiments, the disease is Ornithine Transcarbamylase Deficiency (OTC deficiency). In some embodiments, the disease is Cystic Fibrosis (CF). In some embodiments, the disease is autoimmune hepatitis. In some embodiments, the disease is a hepatic viral disease (e.g., hepatitis A, B, C, D, E). In some embodiments, the disease is hepatitis B. In some embodiments, the disease is Non-alcoholic steatohepatitis (NASH). In some embodiments, a method of expressing a protein or polypeptide of interest in a cell is provided comprising contacting the cell with a lipid composition of the present disclosure.

In some embodiments, a vaccine is provided comprising a lipid composition of claim of the present disclosure. In some embodiments, the vaccine is a SARS-CoV-2 vaccine. In some embodiments, the vaccine is an influenza vaccine. In some embodiments, the vaccine an HIV vaccine. In some embodiments, the vaccine an ebola vaccine. In some embodiments, the vaccine a cancer vaccine.

In one embodiment, a method of inducing an immune response in a subject is provided comprising administering the vaccine to the subject. Thus, in some embodiments a method of preventing a disease in a subject is provided, the method comprising administering a lipid composition provided herein including embodiments thereof to the subject.

In some embodiments, a method of inhibiting expression of a gene or messenger RNA of interest in a cell is provided, comprising contacting the cell with a lipid composition of the disclosure.

In some embodiments, a method of expressing a protein or polypeptide of interest in a subject deficient in the protein or polypeptide of interest is provided comprising administering to the subject a lipid composition of the disclosure, wherein the lipid composition comprise an mRNA that encodes the protein or polypeptide of interest.

For the methods provided herein, in some embodiments, the lipid composition is administered intravenously or intramuscularly. In some embodiments, the lipid composition is administered intravenously. In some embodiments, the lipid composition is administered intramuscularly.

In some embodiments, a method of editing a gene in a cell comprising contacting the cell with a lipid composition of the present disclosure comprising an mRNA, wherein the mRNA encodes a gene-editing enzyme. In some embodiments, the gene editing enzyme comprises one or more components required for gene editing by Zinc finger nucleases (ZFNs), transcription activator like effector nucleases (TALEN), meganucleases, or clustered regularly interspaced short palindromic repeats system (CRISPR/Cas). In some embodiments, the gene editing enzyme is a Zinc finger nuclease (ZFN), transcription activator like effector nuclease (TALEN), meganuclease, or a clustered regularly interspaced short palindromic repeats system (CRISPR/Cas). In some embodiments, the gene-editing enzyme is a TALEN enzyme.

In some embodiments, a method of delivering a nucleic acid into a cell is provided, the method comprising contacting the cell with a lipid composition provided herein, including embodiments thereof.

In some embodiments, a method of imaging a cell is provided, the method comprising contacting a cell with a lipid composition provided herein including embodiments thereof, wherein the nucleic acid encodes a detectable protein.

In embodiments, a method of making a lipid composition provided herein including embodiments thereof, the method including i) contacting the lipid the nucleic acid with the peptide-lipid conjugate, and ii) allowing the peptide-lipid conjugate to encapsulate the nucleic acid.

### Lipid-Based Formulations

Therapies based on the intracellular delivery of nucleic acids to target cells face both extracellular and intracellular barriers. Indeed, naked nucleic acid materials cannot be easily systemically administered due to their toxicity, low stability in serum, rapid renal clearance, reduced uptake by target cells, phagocyte uptake and their ability in activating the immune response, all features that preclude their clinical development. When exogenous nucleic acid material (e.g., mRNA) enters the human biological system, it is recognized by the reticuloendothelial system (RES) as foreign pathogens and cleared from blood circulation before having the chance to encounter target cells within or outside the vascular system. It has been reported that the half-life of naked nucleic acid in the blood stream is around several minutes (Kawabata K, Takakura Y, Hashida MPharm Res. 1995 June; 12(6):825-30). Chemical modification and a proper delivery method can reduce uptake by the RES and protect nucleic acids from degradation by ubiquitous nucleases, which increase stability and efficacy of nucleic acid-based therapies. In addition, RNAs or DNAs are anionic hydrophilic polymers that are not favorable for uptake by cells, which are also anionic at the surface. The success of nucleic acid-based therapies thus depends largely on the development of vehicles or vectors that can efficiently and effectively deliver genetic material to target cells and obtain sufficient levels of expression in vivo with minimal toxicity.

Moreover, upon internalization into a target cell, nucleic acid delivery vectors are challenged by intracellular barriers, including endosome entrapment, lysosomal degradation, nucleic acid unpacking from vectors, translocation across the nuclear membrane (for DNA), and release at the cytoplasm (for RNA). Successful nucleic acid-based therapy thus depends upon the ability of the vector to deliver the nucleic acids to the target sites inside of the cells in order to obtain sufficient levels of a desired activity such as expression of a gene.

While several gene therapies have been able to successfully utilize a viral delivery vector (e.g., AAV), lipid-based formulations have been increasingly recognized as one of the most promising delivery systems for RNA and other nucleic acid compounds due to their biocompatibility and their ease of large-scale production. One of the most significant advances in lipid-based nucleic acid therapies happened in August 2018 when Patisiran (ALN-TTR02) was the first siRNA therapeutic approved by the Food and Drug Administration (FDA) and by the European Commission (EC). ALN-TTR02 is an siRNA formulation based upon the so-called Stable Nucleic Acid Lipid Particle (SNALP) transfecting technology. Despite the success of Patisiran, the delivery of nucleic acid therapeutics, including mRNA, via lipid formulations is still undergoing development.

Some art-recognized lipid-formulated delivery vehicles for nucleic acid therapeutics include, according to various embodiments, polymer based carriers, such as polyethyleneimine (PEI), lipid nanoparticles and liposomes, nanoliposomes, ceramide-containing nanoliposomes, multivesicular liposomes, proteoliposomes, both natural and synthetically-derived exosomes, natural, synthetic and semi-synthetic lamellar bodies, nanoparticulates, micelles, and emulsions. These lipid formulations can vary in their structure and composition, and as can be expected in a rapidly evolving field, several different terms have been used in the art to describe a single type of delivery vehicle. At the same time, the terms for lipid formulations have varied as to their intended meaning throughout the scientific literature, and this inconsistent use has caused confusion as to the exact meaning of several terms for lipid formulations. Among the several potential lipid formulations, liposomes, cationic liposomes, and lipid nanoparticles are specifically described in detail and defined herein for the purposes of the present disclosure.

### Liposomes

Conventional liposomes are vesicles that consist of at least one bilayer and an internal aqueous compartment. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). They generally present as spherical vesicles and can range in size from 20 nm to a few microns. Liposomal formulations can be prepared as a colloidal dispersion or they can be lyophilized to reduce stability risks and to improve the shelf-life for liposome-based drugs. Methods of preparing liposomal compositions are known in the art and are within the skill of an ordinary artisan.

Liposomes that have only one bilayer are referred to as being unilamellar, and those having more than one bilayer are referred to as multilamellar. The most common types of liposomes are small unilamellar vesicles (SUV), large unilamellar vesicles (LUV), and multilamellar vesicles (MLV). In contrast to liposomes, lysosomes, micelles, and reversed micelles are composed of monolayers of lipids. Generally, a liposome is thought of as having a single interior compartment, however some formulations can be multivesicular liposomes (MVL), which consist of numerous discontinuous internal aqueous compartments separated by several nonconcentric lipid bilayers.

Liposomes have long been perceived as drug delivery vehicles because of their superior biocompatibility, given that liposomes are basically analogs of biological membranes, and can be prepared from both natural and synthetic phospholipids (Int. J. Nanomedicine. 2014; 9:1833-1843). In their use as drug delivery vehicles, because a liposome has an aqueous solution core surrounded by a hydrophobic membrane, hydrophilic solutes dissolved in the core cannot readily pass through the bilayer, and hydrophobic compounds will associate with the bilayer. Thus, a liposome can be loaded with hydrophobic and/or hydrophilic molecules. When a liposome is used to carry a nucleic acid such as RNA, the nucleic acid is contained within the liposomal compartment in an aqueous phase.

### Cationic Liposomes

Liposomes can be composed of cationic, anionic, and/or neutral lipids. As an important subclass of liposomes, cationic liposomes are liposomes that are made in whole or part from positively charged lipids, or more specifically a lipid that comprises both a cationic group and a lipophilic portion. In addition to the general characteristics profiled above for liposomes, the positively charged moieties of cationic lipids used in cationic liposomes provide several advantages and some unique structural features. For example, the lipophilic portion of the cationic lipid is hydrophobic and thus will direct itself away from the aqueous interior of the liposome and associate with other nonpolar and hydrophobic species. Conversely, the cationic moiety will associate with aqueous media and more importantly with polar molecules and species with which it can complex in the aqueous interior of the cationic liposome. For these reasons, cationic liposomes are increasingly being researched for use in gene therapy due to their favorability towards negatively charged nucleic acids via electrostatic interactions, resulting in complexes that offer biocompatibility, low toxicity, and the possibility of the large-scale production required for in vivo clinical applications. Cationic lipids suitable for use in cationic liposomes are listed hereinbelow.

### Lipid Nanoparticles

In contrast to liposomes and cationic liposomes, lipid nanoparticles (LNP) have a structure that includes a single monolayer or bilayer of lipids that encapsulates a compound in a solid phase. Thus, unlike liposomes, lipid nanoparticles do not have an aqueous phase or other liquid phase in its interior, but rather the lipids from the bilayer or monolayer shell are directly complexed to the internal compound thereby encapsulating it in a solid core. Lipid nanoparticles are typically spherical vesicles having a relatively uniform dispersion of shape and size. While sources vary on what size qualifies a lipid particle as being a nanoparticle, there is some overlap in agreement that a lipid nanoparticle can have a diameter in the range of from 10 nm to 1000 nm. However, more commonly they are considered to be smaller than 120 nm or even 100 nm.

For lipid nanoparticle nucleic acid delivery systems, the lipid shell can be formulated to include an ionizable cationic lipid which can complex to and associate with the negatively charged backbone of the nucleic acid core. Ionizable cationic lipids with apparent pKa values below about 7 have the benefit of providing a cationic lipid for complexing with the nucleic acid's negatively charged backbone and loading into the lipid nanoparticle at pH values below the pKa of the ionizable lipid where it is positively charged. Then, at physiological pH values, the lipid nanoparticle can adopt a relatively neutral exterior allowing for a significant increase in the circulation half-lives of the particles following i.v. administration. In the context of nucleic acid delivery, lipid nanoparticles offer many advantages over other lipid-based nucleic acid delivery systems including high nucleic acid encapsulation efficiency, potent transfection, improved penetration into tissues to deliver therapeutics, and low levels of cytotoxicity and immunogenicity.

Prior to the development of lipid nanoparticle delivery systems for nucleic acids, cationic lipids were widely studied as synthetic materials for delivery of nucleic acid medicines. In these early efforts, after mixing together at physiological pH, nucleic acids were condensed by cationic lipids to form lipid-nucleic acid complexes known as lipoplexes. However, lipoplexes proved to be unstable and characterized by broad size distributions ranging from the submicron scale to a few microns. Lipoplexes, such as the Lipofectamine® reagent, have found considerable utility for in vitro transfection. However, these first-generation lipoplexes have not proven useful in vivo. The large particle size and positive charge (imparted by the cationic lipid) result in rapid plasma clearance, hemolytic and other toxicities, as well as immune system activation.

### Lipid-Nucleic Acid Formulations

A nucleic acid or a pharmaceutically acceptable salt thereof can be incorporated into a lipid formulation (i.e., a lipid-based delivery vehicle).

In the context of the present disclosure, a lipid-based delivery vehicle typically serves to transport a desired nucleic acid (siRNA, plasmid DNA, mRNA, self-replicating RNA, etc.) to a target cell or tissue. The lipid-based delivery vehicle can be any suitable lipid-based delivery vehicle known in the art. In some embodiments, the lipid-based delivery vehicle is a liposome, a cationic liposome, or a lipid nanoparticle containing a nucleic acid. In some embodiments, the lipid-based delivery vehicle comprises a nanoparticle or a bilayer of lipid molecules and a nucleic acid. In some embodiments, the lipid bilayer preferably further comprises a neutral lipid or a polymer. In some embodiments, the lipid formulation preferably comprises a liquid medium. In some embodiments, the formulation preferably further encapsulates a nucleic acid. In some embodiments, the lipid formulation preferably further comprises a nucleic acid and a neutral lipid or a polymer. In some embodiments, the lipid formulation preferably encapsulates the nucleic acid.

The description provides lipid formulations comprising one or more therapeutic nucleic acid molecules encapsulated within the lipid formulation. In some embodiments, the lipid formulation comprises liposomes. In some embodiments, the lipid formulation comprises cationic liposomes. In some embodiments, the lipid formulation comprises lipid nanoparticles.

In some embodiments, the nucleic acid is fully encapsulated within the lipid portion of the lipid formulation such that the nucleic acid in the lipid formulation is resistant in aqueous solution to nuclease degradation. In other embodiments, the lipid formulations described herein are substantially non-toxic to mammals such as humans.

The lipid formulations of the disclosure also typically have a total lipid:nucleic acid ratio (mass/mass ratio) of from about 1:1 to about 100:1, from about 1:1 to about 50:1, from about 2:1 to about 45:1, from about 3:1 to about 40:1, from about 5:1 to about 38:1, or from about 6:1 to about 40:1, or from about 7:1 to about 35:1, or from about 8:1 to about 30:1; or from about 10:1 to about 25:1; or from about 8:1 to about 12:1; or from about 13:1 to about 17:1; or from about 18:1 to about 24:1; or from about 20:1 to about 30:1. In some preferred embodiments, the total lipid:nucleic acid ratio (mass/mass ratio) is from about 10:1 to about 25:1. The ratio may be any value or subvalue within the recited ranges, including endpoints.

The lipid formulations of the present disclosure typically have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, or about 150 nm, and are substantially non-toxic. The diameter may be any value or subvalue within the recited ranges, including endpoints. In addition, nucleic acids, when present in the lipid nanoparticles of the present disclosure, are resistant in aqueous solution to degradation with a nuclease.

In preferred embodiments, the lipid formulations comprise a nucleic acid, a cationic lipid (e.g., one or more cationic lipids or salts thereof described herein), a phospholipid, and a conjugated lipid that inhibits aggregation of the particles (e.g., one or more PEG-lipid conjugate and/or a peptide-lipid conjugate of the disclosure). The lipid formulations can also include cholesterol.

In the nucleic acid-lipid formulations, the nucleic acid may be fully encapsulated within the lipid portion of the formulation, thereby protecting the nucleic acid from nuclease degradation.

In preferred embodiments, a lipid formulation comprising a nucleic acid is fully encapsulated within the lipid portion of the lipid formulation, thereby protecting the nucleic acid from nuclease degradation. In certain instances, the nucleic acid in the lipid formulation is not substantially degraded after exposure of the particle to a nuclease at 37° C. for at least 20, 30, 45, or 60 minutes. In certain other instances, the nucleic acid in the lipid formulation is not substantially degraded after incubation of the formulation in serum at 37° C. for at least 30, 45, or 60 minutes or at least 2, 3, 4, 5, 6, 7, 8, 9, 10, 12, 14, 16, 18, 20, 22, 24, 26, 28, 30, 32, 34, or 36 hours. In other embodiments, the nucleic acid is complexed with the lipid portion of the formulation.

In the context of nucleic acids, full encapsulation may be determined by performing a membrane-impermeable fluorescent dye exclusion assay, which uses a dye that has enhanced fluorescence when associated with nucleic acid. Encapsulation is determined by adding the dye to a lipid formulation, measuring the resulting fluorescence, and comparing it to the fluorescence observed upon addition of a small amount of nonionic detergent. Detergent-mediated disruption of the lipid layer releases the encapsulated nucleic acid, allowing it to interact with the membrane-impermeable dye. Nucleic acid encapsulation may be calculated as E=(I0−I)/I0, where I and I0 refer to the fluorescence intensities before and after the addition of detergent.

In other embodiments, the present disclosure provides a nucleic acid-lipid composition comprising a plurality of nucleic acid-liposomes, nucleic acid-cationic liposomes, or nucleic acid-lipid nanoparticles. In some embodiments, the nucleic acid-lipid composition comprises a plurality of nucleic acid-liposomes. In some embodiments, the nucleic acid-lipid composition comprises a plurality of nucleic acid-cationic liposomes. In some embodiments, the nucleic acid-lipid composition comprises a plurality of nucleic acid-lipid nanoparticles.

In some embodiments, the lipid formulations comprise a nucleic acid that is fully encapsulated within the lipid portion of the formulation, such that from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% (or any fraction thereof or range therein) of the particles have the nucleic acid encapsulated therein. The amount may be any value or subvalue within the recited ranges, including endpoints.

Depending on the intended use of the lipid formulation, the proportions of the components can be varied, and the delivery efficiency of a particular formulation can be measured using assays known in the art.

According to some embodiments, expressible polynucleotides, nucleic acid active agents, and mRNA constructs can be lipid formulated. The lipid formulation is preferably selected from, but not limited to, liposomes, cationic liposomes, and lipid nanoparticles. In one preferred embodiment, a lipid formulation is a cationic liposome or a lipid nanoparticle (LNP) comprising:
(a) a nucleic acid (mRNA, siRNA, etc.),
(b) a cationic lipid,
(c) a peptide-lipid conjugate of the disclosure,
(d) optionally a non-cationic lipid (such as a neutral lipid), and
(e) optionally, a sterol.

In one some embodiments, the cationic lipid is an ionizable cationic lipid. In one embodiment, the lipid nanoparticle formulation consists of (i) at least one cationic lipid; (ii) a helper lipid; (iii) a sterol (e.g., cholesterol); and (iv) a peptide-lipid conjugate of the disclosure, in a molar ratio of about 20% to about 40% ionizable cationic lipid: about 25% to about 45% helper lipid: about 25% to about 45% sterol; about 0.5-5% peptide lipid conjugate. Example cationic lipids (including ionizable cationic lipids), helper lipids (e.g., neutral lipids), and sterols are described hereinbelow.

### Cationic Lipids

The lipid formulation preferably includes a cationic lipid suitable for forming a cationic liposome or lipid nanoparticle. Cationic lipids are widely studied for nucleic acid delivery because they can bind to negatively charged membranes and induce uptake. Generally, cationic lipids are amphiphiles containing a positive hydrophilic head group, two (or more) lipophilic tails, or a steroid portion and a connector between these two domains. Preferably, the cationic lipid carries a net positive charge at about physiological pH. Cationic liposomes have been traditionally the most commonly used non-viral delivery systems for oligonucleotides, including plasmid DNA, antisense oligos, and siRNA/small hairpin RNA-shRNA. Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids by electrostatic interaction, providing high in vitro transfection efficiency.

In the presently disclosed lipid formulations, the cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (7-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanediol (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,3 1-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination thereof. Other cationic lipids include, but are not limited to, N,N-distearyl-N,N-dimethylammonium bromide (DDAB), 3P-(N—(N,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., PNAS, 107(5), 1864-69, 2010, the contents of which are herein incorporated by reference.

Other suitable cationic lipids include those having alternative fatty acid groups and other dialkylamino groups, including those, in which the alkyl substituents are different (e.g., N-ethyl-N-methylamino-, and N-propyl-N-ethylamino-). These lipids are part of a subcategory of cationic lipids referred to as amino lipids. In some embodiments of the lipid formulations described herein, the cationic lipid is an amino lipid. In general, amino lipids having less saturated alkyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

In some embodiments, the lipid formulation comprises the cationic lipid with Formula I according to the patent application PCT/EP2017/064066. In this context, the disclosure of PCT/EP2017/064066 is also incorporated herein by reference.

In some embodiments, amino or cationic lipids of the present disclosure are ionizable and have at least one protonatable or deprotonatable group, such that the lipid is positively charged at a pH at or below physiological pH (e.g., pH 7.4), and neutral at a second pH, preferably at or above physiological pH. Of course, it will be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11. In some embodiments, the ionizable cationic lipid has a pKa of about 5 to about 7. In some embodiments, the pKa of an ionizable cationic lipid is about 6 to about 7.

In some embodiments, the lipid formulation comprises an ionizable cationic lipid of Formula I:

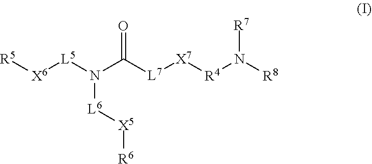

or a pharmaceutically acceptable salt or solvate thereof, wherein R⁵ and R⁶ are each independently selected from the group consisting of a linear or branched C₁₋C₃₁ alkyl, C₂₋C₃₁ alkenyl or C₂₋C₃₁ alkynyl and cholesteryl; L⁵ and L⁶ are each independently selected from the group consisting of a linear C₁₋C₂₀ alkyl and C₂₋C₂₀ alkenyl; X⁵ is —C(O)O—, whereby —C(O)O—R⁶ is formed or —OC(O)— whereby —OC(O)—R⁶ is formed; X⁶ is —C(O)O— whereby —C(O)O—R⁵ is formed or —OC(O)— whereby —OC(O)—R⁵ is formed; X⁷ is S or O; L⁷ is absent or lower alkyl; R⁴ is a linear or branched C₁-C₆ alkyl; and R⁷ and R⁸ are each independently selected from the group consisting of a hydrogen and a linear or branched C₁-C₆ alkyl.

In some embodiments, X⁷ is S.

In some embodiments, X⁵ is —C(O)O—, whereby —C(O)O—R⁶ is formed and X⁶ is —C(O)O— whereby —C(O)O—R⁵ is formed.

In some embodiments, R⁷ and R⁸ are each independently selected from the group consisting of methyl, ethyl and isopropyl.

In some embodiments, L⁵ and L⁶ are each independently a C₁-C₁₀ alkyl. In some embodiments, L⁵ is C₁-C₃ alkyl, and L6 is C₁-C₅ alkyl. In some embodiments, L⁶ is C₁-C₂ alkyl. In some embodiments, L⁵ and L⁶ are each a linear C₇ alkyl. In some embodiments, L⁵ and L⁶ are each a linear C₉ alkyl.

In some embodiments, R⁵ and R⁶ are each independently an alkenyl. In some embodiments, R⁶ is alkenyl. In some embodiments, R⁶ is C₂-C₉ alkenyl. In some embodiments, the alkenyl comprises a single double bond. In some embodiments, R⁵ and R⁶ are each alkyl. In some embodiments, R⁵ is a branched alkyl. In some embodiments, R⁵ and R⁶ are each independently selected from the group consisting of a C₉ alkyl, C₉ alkenyl and C₉ alkynyl. In some embodiments, R⁵ and R⁶ are each independently selected from the group consisting of a Cu alkyl, C₁₁ alkenyl and C₁₁ alkynyl. In some embodiments, R⁵ and R⁶ are each independently selected from the group consisting of a C₇ alkyl, C₇ alkenyl and C₇ alkynyl. In some embodiments, R⁵ is —CH((CH₂)ₚCH₃)₂ or —CH((CH₂)ₚCH₃)((CH₂)ₚ₋₁CH₃), wherein p is 4-8. In some embodiments, p is 5 and L⁵ is a C₁-C₃ alkyl. In some embodiments, p is 6 and L⁵ is a C₃ alkyl. In some embodiments, p is 7. In some embodiments, p is 8 and L⁵ is a C₁-C₃ alkyl. In some embodiments, R⁵ consists of —CH((CH₂)ₚCH₃)((CH₂)ₚ₋₁CH₃), wherein p is 7 or 8.

In some embodiments, R⁴ is ethylene or propylene. In some embodiments, R⁴ is n-propylene or isobutylene.

In some embodiments, L⁷ is absent, R⁴ is ethylene, X⁷ is S and R⁷ and R⁸ are each methyl. In some embodiments, L⁷ is absent, R⁴ is n-propylene, X⁷ is S and R⁷ and R⁸ are each methyl. In some embodiments, L⁷ is absent, R⁴ is ethylene, X⁷ is S and R⁷ and R⁸ are each ethyl.

In some embodiments, X⁷ is S, X⁵ is —C(O)O—, whereby —C(O)O—R⁶ is formed, X⁶ is —C(O)O— whereby —C(O)O—R⁵ is formed, L⁵ and L⁶ are each independently a linear C₃-C₇ alkyl, L⁷ is absent, R⁵ is —CH((CH₂)ₚCH₃)₂, and R⁶ is C₇-C₁₂ alkenyl. In some further embodiments, p is 6 and R⁶ is C₉ alkenyl.

In some embodiments, the lipid formulation comprises an ionizable cationic lipid selected from the group ATX lipids disclosed hereinabove.

In some embodiments, any one or more lipids recited herein may be expressly excluded.

### Helper Lipids and Sterols

The mRNA-lipid formulations of the present disclosure can comprise a helper lipid, which can be referred to as a neutral lipid, a neutral helper lipid, non-cationic lipid, non-cationic helper lipid, anionic lipid, anionic helper lipid, or a zwitterionic lipid. It has been found that lipid formulations, particularly cationic liposomes and lipid nanoparticles have increased cellular uptake if helper lipids are present in the formulation. (Curr. Drug Metab. 2014; 15(9):882-92). For example, some studies have indicated that neutral and zwitterionic lipids such as 1,2-dioleoyl-sn-glycero-3-phosphatidylcholine (DOPC), Di-Oleoyl-Phosphatidyl-Ethanoalamine (DOPE) and 1,2-DiStearoyl-sn-glycero-3-PhosphoCholine (DSPC), being more fusogenic (i.e., facilitating fusion) than cationic lipids, can affect the polymorphic features of lipid-nucleic acid complexes, promoting the transition from a lamellar to a hexagonal phase, and thus inducing fusion and a disruption of the cellular membrane. (Nanomedicine (Lond). 2014 January; 9(1):105-20). In addition, the use of helper lipids can help to reduce any potential detrimental effects from using many prevalent cationic lipids such as toxicity and immunogenicity.

Non-limiting examples of non-cationic lipids suitable for lipid formulations of the present disclosure include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl.

Additional examples of non-cationic lipids include sterols such as cholesterol and derivatives thereof. One study concluded that as a helper lipid, cholesterol increases the spacing of the charges of the lipid layer interfacing with the nucleic acid making the charge distribution match that of the nucleic acid more closely. (J. R. Soc. Interface. 2012 Mar. 7; 9(68): 548-561). Non-limiting examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In preferred embodiments, the cholesterol derivative is a polar analogue such as cholesteryl-(4′-hydroxy)-butyl ether.

In some embodiments, the helper lipid present in the lipid formulation comprises or consists of a mixture of one or more phospholipids and cholesterol or a derivative thereof. In other embodiments, the helper lipid present in the lipid formulation comprises or consists of one or more phospholipids, e.g., a cholesterol-free lipid formulation. In yet other embodiments, the helper lipid present in the lipid formulation comprises or consists of cholesterol or a derivative thereof, e.g., a phospholipid-free lipid formulation.

Other examples of helper lipids include nonphosphorous containing lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyldimethyl ammonium bromide, ceramide, and sphingomyelin.

In some embodiments, the helper lipid comprises from about 20 mol % to about 50 mol %, from about 22 mol % to about 48 mol %, from about 24 mol % to about 46 mol %, about 25 mol % to about 44 mol %, from about 26 mol % to about 42 mol %, from about 27 mol % to about 41 mol %, from about 28 mol % to about 40 mol %, or about 29 mol %, about 30 mol %, about 31 mol %, about 32 mol %, about 33 mol %, about 34 mol %, about 35 mol %, about 36 mol %, about 37 mol %, about 38 mol %, or about 39 mol % (or any fraction thereof or the range therein) of the total lipid present in the lipid formulation.

In some embodiments, the total of helper lipid in the formulation comprises two or more helper lipids and the total amount of helper lipid comprises from about 20 mol % to about 50 mol %, from about 22 mol % to about 48 mol %, from about 24 mol % to about 46 mol %, about 25 mol % to about 44 mol %, from about 26 mol % to about 42 mol %, from about 27 mol % to about 41 mol %, from about 28 mol % to about 40 mol %, or about 29 mol %, about 30 mol %, about 31 mol %, about 32 mol %, about 33 mol %, about 34 mol %, about 35 mol %, about 36 mol %, about 37 mol %, about 38 mol %, or about 39 mol % (or any fraction thereof or the range therein) of the total lipid present in the lipid formulation. In some embodiments, the helper lipids are a combination of DSPC and DOTAP. In some embodiments, the helper lipids are a combination of DSPC and DOTMA.

The cholesterol or cholesterol derivative in the lipid formulation may comprise up to about 40 mol %, about 45 mol %, about 50 mol %, about 55 mol %, or about 60 mol % of the total lipid present in the lipid formulation. In some embodiments, the cholesterol or cholesterol derivative comprises about 15 mol % to about 45 mol %, about 20 mol % to about 40 mol %, about 30 mol % to about 40 mol %, or about 35 mol %, about 36 mol %, about 37 mol %, about 38 mol %, about 39 mol %, or about 40 mol % of the total lipid present in the lipid formulation.

The percentage of helper lipid present in the lipid formulation is a target amount, and the actual amount of helper lipid present in the formulation may vary, for example, by ±5 mol %.

A lipid formulation containing a cationic lipid compound or ionizable cationic lipid compound may be on a molar basis about 20-40% cationic lipid compound, about 25-40% cholesterol, about 25-50% helper lipid, and about 0.5-5% of a peptide-lipid conjugate of the disclosure, wherein the percent is of the total lipid present in the formulation. In some embodiments, the composition is about 22-30% cationic lipid compound, about 30-40% cholesterol, about 30-40% helper lipid, and about 0.5-3% of a peptide-lipid conjugate of the disclosure, wherein the percent is of the total lipid present in the formulation.

### Lipid Conjugates

In some embodiments, one or more peptide-lipid conjugates of the present disclosure comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 0.9 mol % to about 1.6 mol %, from about 0.9 mol % to about 1.8 mol %, from about 1 mol % to about 1.8 mol %, from about 1 mol % to about 1.7 mol %, from about 1.2 mol % to about 1.8 mol %, from about 1.2 mol % to about 1.7 mol %, from about 1.3 mol % to about 1.6 mol %, or from about 1.4 mol % to about 1.6 mol % (or any fraction thereof or range therein) of the total lipid present in the lipid formulation. In other embodiments, one or more peptide-lipid conjugates comprise about 0.5%, 0.6%, 0.7%, 0.8%, 0.9%, 1.0%, 1.2%, 1.3%, 1.4%, 1.5%, 1.6%, 1.7%, 1.8%, 1.9%, 2.0%, 2.5%, 3.0%, 3.5%, 4.0%, 4.5%, or 5%, (or any fraction thereof or range therein) of the total lipid present in the lipid formulation. The amount may be any value or subvalue within the recited ranges, including endpoints.

The percentage of peptide-lipid conjugate present in the lipid formulations of the disclosure is a target amount, and the actual amount of peptide-lipid conjugate present in the formulation may vary, for example, by ±0.5 mol %. One of ordinary skill in the art will appreciate that the concentration of the lipid conjugate can be varied depending on the lipid conjugate employed and the rate at which the lipid formulation is to become fusogenic.

### Mechanism of Action for Cellular Uptake of Lipid Formulations

Lipid formulations for the intracellular delivery of nucleic acids, particularly liposomes, cationic liposomes, and lipid nanoparticles, are designed for cellular uptake by penetrating target cells through exploitation of the target cells' endocytic mechanisms where the contents of the lipid delivery vehicle are delivered to the cytosol of the target cell. (Nucleic Acid Therapeutics, 28(3):146-157, 2018). Specifically, in the case of a nucleic acid-lipid formulations described herein, the lipid formulation enters cells through receptor mediated endocytosis. Prior to endocytosis, functionalized ligands such as a peptide-lipid conjugate of the disclosure at the surface of the lipid delivery vehicle can be shed from the surface, which triggers internalization into the target cell. During endocytosis, some part of the plasma membrane of the cell surrounds the vector and engulfs it into a vesicle that then pinches off from the cell membrane, enters the cytosol and ultimately undergoes the endolysosomal pathway. For ionizable cationic lipid-containing delivery vehicles, the increased acidity as the endosome ages results in a vehicle with a strong positive charge on the surface. Interactions between the delivery vehicle and the endosomal membrane then result in a membrane fusion event that leads to cytosolic delivery of the payload. For mRNA or self-replicating RNA payloads, the cell's own internal translation processes will then translate the RNA into the encoded protein. The encoded protein can further undergo post-translational processing, including transportation to a targeted organelle or location within the cell.

By controlling the composition and concentration of the lipid conjugate, one can control the rate at which the lipid conjugate exchanges out of the lipid formulation and, in turn, the rate at which the lipid formulation becomes fusogenic. In addition, other variables including, e.g., pH, temperature, or ionic strength, can be used to vary and/or control the rate at which the lipid formulation becomes fusogenic. Other methods which can be used to control the rate at which the lipid formulation becomes fusogenic will become apparent to those of skill in the art upon reading this disclosure. Also, by controlling the composition and concentration of the lipid conjugate, one can control the liposomal or lipid particle size.

### Lipid Formulation Manufacture

There are many different methods for the preparation of lipid formulations comprising a nucleic acid. (Curr. Drug Metabol. 2014, 15, 882-892; Chem. Phys. Lipids 2014, 177, 8-18; Int. J. Pharm. Stud. Res. 2012, 3, 14-20). The techniques of thin film hydration, double emulsion, reverse phase evaporation, microfluidic preparation, dual asymmetric centrifugation, ethanol injection, detergent dialysis, spontaneous vesicle formation by ethanol dilution, and encapsulation in preformed liposomes are briefly described herein.

### Thin Film Hydration

In Thin Film Hydration (TFH) or the Bangham method, the lipids are dissolved in an organic solvent, then evaporated through the use of a rotary evaporator leading to a thin lipid layer formation. After the layer hydration by an aqueous buffer solution containing the compound to be loaded, Multilamellar Vesicles (MLVs) are formed, which can be reduced in size to produce Small or Large Unilamellar vesicles (LUV and SUV) by extrusion through membranes or by the sonication of the starting MLV.

### Double Emulsion

Lipid formulations can also be prepared through the Double Emulsion technique, which involves lipids dissolution in a water/organic solvent mixture. The organic solution, containing water droplets, is mixed with an excess of aqueous medium, leading to a water-in-oil-in-water (W/O/W) double emulsion formation. After mechanical vigorous shaking, part of the water droplets collapse, giving Large Unilamellar Vesicles (LUVs).

### Reverse Phase Evaporation

The Reverse Phase Evaporation (REV) method also allows one to achieve LUVs loaded with nucleic acid. In this technique a two-phase system is formed by phospholipids dissolution in organic solvents and aqueous buffer. The resulting suspension is then sonicated briefly until the mixture becomes a clear one-phase dispersion. The lipid formulation is achieved after the organic solvent evaporation under reduced pressure. This technique has been used to encapsulate different large and small hydrophilic molecules including nucleic acids.

### Microfluidic Preparation

The Microfluidic method, unlike other bulk techniques, gives the possibility of controlling the lipid hydration process. The method can be classified in continuous-flow microfluidic and droplet-based microfluidic, according to the way in which the flow is manipulated. In the microfluidic hydrodynamic focusing (MIF) method, which operates in a continuous flow mode, lipids are dissolved in isopropyl alcohol which is hydrodynamically focused in a microchannel cross junction between two aqueous buffer streams. Vesicles size can be controlled by modulating the flow rates, thus controlling the lipids solution/buffer dilution process. The method can be used for producing oligonucleotide (ON) lipid formulations by using a microfluidic device consisting of three-inlet and one-outlet ports.

### Dual Asymmetric Centrifugation

Dual Asymmetric Centrifugation (DAC) differs from more common centrifugation as it uses an additional rotation around its own vertical axis. An efficient homogenization is achieved due to the two overlaying movements generated: the sample is pushed outwards, as in a normal centrifuge, and then it is pushed towards the center of the vial due to the additional rotation. By mixing lipids and an NaCl-solution a viscous vesicular phospholipid gel (VPC) is achieved, which is then diluted to obtain a lipid formulation dispersion. The lipid formulation size can be regulated by optimizing DAC speed, lipid concentration and homogenization time.

### Ethanol Injection

The Ethanol Injection (EI) method can be used for nucleic acid encapsulation. This method provides the rapid injection of an ethanolic solution, in which lipids are dissolved, into an aqueous medium containing nucleic acids to be encapsulated, through the use of a needle. Vesicles are spontaneously formed when the phospholipids are dispersed throughout the medium.

### Detergent Dialysis

The Detergent dialysis method can be used to encapsulate nucleic acids. Briefly lipid and plasmid are solubilized in a detergent solution of appropriate ionic strength, after removing the detergent by dialysis, a stabilized lipid formulation is formed. Unencapsulated nucleic acid is then removed by ion-exchange chromatography and empty vesicles by sucrose density gradient centrifugation. The technique is highly sensitive to the cationic lipid content and to the salt concentration of the dialysis buffer, and the method is also difficult to scale.

### Spontaneous Vesicle Formation by Ethanol Dilution

Stable lipid formulations can also be produced through the Spontaneous Vesicle Formation by Ethanol Dilution method in which a stepwise or dropwise ethanol dilution provides the instantaneous formation of vesicles loaded with nucleic acid by the controlled addition of lipid dissolved in ethanol to a rapidly mixing aqueous buffer containing the nucleic acid.

### Pharmaceutical Compositions and Delivery Methods

To facilitate nucleic acid activity (e.g., mRNA expression, or knockdown by an ASO or siRNA) in vivo, the nucleic acid lipid formulation delivery vehicles described herein can be combined with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition.

The lipid formulations and pharmaceutical compositions of the present disclosure may be administered and dosed in accordance with current medical practice, taking into account the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein (e.g., enzyme) production. Another example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations.

The pharmaceutical compositions described herein can be an inhalable composition. Suitable routes of administration include, for example, intratracheal, inhaled, or intranasal. In some embodiments, the administration results in delivery of the nucleic acid to a lung epithelial cell. In some embodiments, the administration shows a selectivity towards lung epithelial cells over other types of lung cells and cells of the airways.

The pharmaceutical compositions disclosed herein can be formulated using one or more excipients to: (1) increase stability; (2) increase cell transfection; (3) permit a sustained or delayed release (e.g., from a depot formulation of the nucleic acid); (4) alter the biodistribution (e.g., target the nucleic acid to specific tissues or cell types); (5) increase the activity of the nucleic acid or a protein expressed therefrom in vivo; and/or (6) alter the release profile of the nucleic acid or an encoded protein in vivo.

Preferably, the lipid formulations may be administered in a local rather than systemic manner. Local delivery can be affected in various ways, depending on the tissue to be targeted. For example, aerosols containing compositions of the present disclosure can be inhaled (for nasal, tracheal, or bronchial delivery).

Pharmaceutical compositions may be administered to any desired tissue. In some embodiments, the nucleic acid delivered by a lipid formulation or composition of the present disclosure is active in the tissue in which the lipid formulation and/or composition was administered. In some embodiments, the nucleic acid is active in a tissue different from the tissue in which the lipid formulation and/or composition was administered. Example tissues in which the nucleic acid may be delivered include, but are not limited to the lung, trachea, and/or nasal passages, muscle, liver, eye, or the central nervous system.

The pharmaceutical compositions described herein may be prepared by any method known or hereafter developed in the art of pharmacology. In general, such preparatory methods include the step of associating the active ingredient (i.e., nucleic acid) with an excipient and/or one or more other accessory ingredients. A pharmaceutical composition in accordance with the present disclosure may be prepared, packaged, and/or sold in bulk, as a single unit dose, and/or as a plurality of single unit doses.

Pharmaceutical compositions may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with a primary DNA construct, or mRNA (e.g., for transplantation into a subject), hyaluronidase, nanoparticle mimics and combinations thereof.

Accordingly, the formulations described herein can include one or more excipients, each in an amount that together increases the stability of the nucleic acid in the lipid formulation, increases cell transfection by the nucleic acid (e.g., mRNA or siRNA), increases the expression of an encoded protein, and/or alters the release profile of the encoded protein, or increases knockdown of a target native nucleic acid. Further, a nucleic acid may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

A dosage form of the composition of this disclosure can be solid, which can be reconstituted in a liquid prior to administration. The solid can be administered as a powder. In some embodiments, the pharmaceutical composition comprises a nucleic acid lipid formulation that has been lyophilized.

In a preferred embodiment, the dosage form of the pharmaceutical compositions described herein can be a liquid suspension of nucleic acid-lipid nanoparticles described herein. In some embodiments, the liquid suspension is in a buffered solution. In some embodiments, the buffered solution comprises a buffer selected from the group consisting of HEPES, MOPS, TES, and TRIS. In some embodiments, the buffer has a pH of about 7.4. In some preferred embodiments, the buffer is HEPES. In some further embodiments, the buffered solution further comprises a cryoprotectant. In some embodiments, the cryoprotectant is selected from a sugar and glycerol or a combination of a sugar and glycerol. In some embodiments, the sugar is a dimeric sugar. In some embodiments, the sugar is sucrose. In some preferred embodiments, the buffer comprises HEPES, sucrose, and glycerol at a pH of 7.4. In some embodiments, the suspension is frozen during storage and thawed prior to administration. In some embodiments, the suspension is frozen at a temperature below about −70° C. In some embodiments, the suspension is diluted with sterile water prior to inhalable administration. In some embodiments, an inhalable administration comprises diluting the suspension with about 1 volume to about 4 volumes of sterile water. In some embodiments, a lyophilized nucleic acid-lipid nanoparticle formulation can be resuspended in a buffer as described herein.

The compositions and methods of the disclosure may be administered to subjects by a variety of mucosal administration modes, including intranasal and/or intrapulmonary. In some aspects of this disclosure, the mucosal tissue layer includes an epithelial cell layer. The epithelial cell can be pulmonary, tracheal, bronchial, alveolar, nasal, and/or buccal. Compositions of this disclosure can be administered using conventional actuators such as mechanical spray devices, as well as pressurized, electrically activated, or other types of actuators.

The compositions of this disclosure may be administered in an aqueous solution as a nasal or pulmonary spray and may be dispensed in spray form by a variety of methods known to those skilled in the art. Pulmonary delivery of a composition of this disclosure is achieved by administering the composition in the form of drops, particles, or spray, which can be, for example, aerosolized, atomized, or nebulized. Particles of the composition, spray, or aerosol can be in either a liquid or solid form, for example, a lyophilized lipid formulation. Preferred systems for dispensing liquids as a nasal spray are disclosed in U.S. Pat. No. 4,511,069. Such formulations may be conveniently prepared by dissolving compositions according to the present disclosure in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in TRANSDERMAL SYSTEMIC MEDICATION, Y. W. Chien ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the nucleic acid-lipid formulation or suspended in a pharmaceutical solvent, e.g., water, ethanol, or mixtures thereof.

Nasal and pulmonary spray solutions of the present disclosure typically comprise the nucleic acid, optionally formulated with a surface-active agent, such as a nonionic surfactant (e.g., polysorbate-80), and one or more buffers, provided that the inclusion of the surfactant does not disrupt the structure of the lipid formulation. In some embodiments of the present disclosure, the nasal spray solution further comprises a propellant. The pH of the nasal spray solution may be from pH 6.8 to 7.2. The pharmaceutical solvents employed can also be a slightly acidic aqueous buffer of pH 4-6. Other components may be added to enhance or maintain chemical stability, including preservatives, surfactants, dispersants, or gases.

In some embodiments, this disclosure provides a pharmaceutical product which includes a solution containing a composition of this disclosure and an actuator for a pulmonary, mucosal, or intranasal spray or aerosol.

A dosage form of the composition of this disclosure can be liquid, in the form of droplets or an emulsion, or in the form of an aerosol.

A dosage form of the composition of this disclosure can be solid, which can be reconstituted in a liquid prior to administration. The solid can be administered as a powder. The solid can be in the form of a capsule, tablet, or gel.

To formulate compositions for pulmonary delivery within the present disclosure, the nucleic acid-lipid formulation can be combined with various pharmaceutically acceptable additives, as well as a base or carrier for dispersion of the nucleic acid-lipid formulation(s).

Examples of additives include pH control agents such as arginine, sodium hydroxide, glycine, hydrochloric acid, citric acid, and mixtures thereof. Other additives include local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione). When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the mucosa at the site of administration. Generally, the tonicity of the solution is adjusted to a value of 1/3 to 3, more typically 1/2 to 2, and most often 3/4 to 1.7.

The nucleic acid-lipid formulation may be dispersed in a base or vehicle, which may comprise a hydrophilic compound having a capacity to disperse the nucleic acid-lipid formulation and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl(meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer, and mixtures thereof. Alternatively or additionally, synthetic fatty acid esters such as polyglycerin fatty acid esters, sucrose fatty acid esters, etc., can be employed as carriers. Hydrophilic polymers and other carriers can be used alone or in combination and enhanced structural integrity can be imparted to the carrier by partial crystallization, ionic bonding, crosslinking, and the like. The carrier can be provided in a variety of forms, including fluid or viscous solutions, gels, pastes, powders, microspheres, and films for direct application to the nasal mucosa. The use of a selected carrier in this context may result in promotion of absorption of the nucleic acid-lipid formulation.

The compositions of this disclosure may alternatively contain as pharmaceutically acceptable carriers substances as required to approximate physiological conditions, such as pH adjusting and buffering agents, tonicity adjusting agents, and wetting agents, for example, sodium acetate, sodium lactate, sodium chloride, potassium chloride, calcium chloride, sorbitan monolaurate, triethanolamine oleate, and mixtures thereof. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the disclosure, the nucleic acid-lipid formulation may be administered in a time release formulation, for example in a composition which includes a slow release polymer. The nucleic acid-lipid formulation can be prepared with carriers that will protect against rapid release, for example a controlled release vehicle such as a polymer, microencapsulated delivery system, or a bioadhesive gel. Prolonged delivery of the nucleic acid-lipid formulation, in various compositions of the disclosure can be brought about by including in the composition agents that delay absorption, for example, aluminum monostearate hydrogels and gelatin.

It has been demonstrated that nucleic acids can be delivered to the lungs by intratracheal administration of a liquid suspension of the nucleic acid composition and inhalation of an aerosol mist produced by a liquid nebulizer or the use of a dry powder apparatus such as that described in U.S. Pat. No. 5,780,014, incorporated herein by reference.

In certain embodiments, the compositions of the disclosure may be formulated such that they may be aerosolized or otherwise delivered as a particulate liquid or solid prior to or upon administration to the subject. Such compositions may be administered with the assistance of one or more suitable devices for administering such solid or liquid particulate compositions (such as, e.g., an aerosolized aqueous solution or suspension) to generate particles that are easily respirable or inhalable by the subject. In some embodiments, such devices (e.g., a metered dose inhaler, jet-nebulizer, ultrasonic nebulizer, dry-powder-inhalers, propellant-based inhaler or an insufflator) facilitate the administration of a predetermined mass, volume or dose of the compositions (e.g., about 0.010 to about 0.5 mg/kg of nucleic acid per dose) to the subject. For example, in certain embodiments, the compositions of the disclosure are administered to a subject using a metered dose inhaler containing a suspension or solution comprising the composition and a suitable propellant. In certain embodiments, the compositions of the disclosure may be formulated as a particulate powder (e.g., respirable dry particles) intended for inhalation. In certain embodiments, compositions of the disclosure formulated as respirable particles are appropriately sized such that they may be respirable by the subject or delivered using a suitable device (e.g., a mean D50 or D90 particle size less than about 500 μm, 400 μm, 300 μm, 250 μm, 200 μm, 150 μm, 100 μm, 75 μm, 50 μm, 25 μm, 20 μm, 15 μm, 12.5 μm, 10 μm, 5 μm, 2.5 μm or smaller). In yet other embodiments, the compositions of the disclosure are formulated to include one or more pulmonary surfactants (e.g., lamellar bodies). In some embodiments, the compositions of the disclosure are administered to a subject such that a concentration of at least 0.010 mg/kg, at least 0.015 mg/kg, at least 0.020 mg/kg, at least 0.025 mg/kg, at least 0.030 mg/kg, at least 0.035 mg/kg, at least 0.040 mg/kg, at least 0.045 mg/kg, at least 0.05 mg/kg, at least 0.1 mg/kg, at least 0.5 mg/kg, at least 1.0 mg/kg, at least 2.0 mg/kg, at least 3.0 mg/kg, at least 4.0 mg/kg, at least 5.0 mg/kg, at least 6.0 mg/kg, at least 7.0 mg/kg, at least 8.0 mg/kg, at least 9.0 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg body weight is administered in a single dose. In some embodiments, the compositions of the disclosure are administered to a subject such that a total amount of at least 0.1 mg, at least 0.5 mg, at least 1.0 mg, at least 2.0 mg, at least 3.0 mg, at least 4.0 mg, at least 5.0 mg, at least 6.0 mg, at least 7.0 mg, at least 8.0 mg, at least 9.0 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg or at least 100 mg nucleic acid is administered in one or more doses.

In some embodiments, a pharmaceutical composition of the present disclosure is administered to a subject once per month. In some embodiments, a pharmaceutical composition of the present disclosure is administered to a subject twice per month. In some embodiments, a pharmaceutical composition of the present disclosure is administered to a subject three times per month. In some embodiments, a pharmaceutical composition of the present disclosure is administered to a subject four times per month.

According to the present disclosure, a therapeutically effective dose of the provided composition, when administered regularly, results in an increased nucleic acid activity level in a subject as compared to a baseline activity level before treatment. Typically, the activity level is measured in a biological sample obtained from the subject such as blood, plasma or serum, urine, or solid tissue extracts. The baseline level can be measured immediately before treatment. In some embodiments, administering a pharmaceutical composition described herein results in an increased nucleic acid activity level in a biological sample (e.g., plasma/serum or lung epithelial swab) by at least about 10%, 20%, 30%, 40%, 50%, 60%, 70%, 80%, 90%, or 95% as compared to a baseline level before treatment. In some embodiments, administering the provided composition results in an increased nucleic acid activity level in a biological sample (e.g., plasma/serum or lung epithelial swab) by at least about 10%, 20%, 30%, 40%, 50%, 60%, 70%, 80%, 90%, or 95% as compared to a baseline level before treatment for at least about 24 hours, at least about 48 hours, at least about 72 hours, at least about 4 days, at least about 5 days, at least about 6 days, at least about 7 days, at least about 8 days, at least about 9 days, at least about 10 days, at least about 11 days, at least about 12 days, at least about 13 days, at least about 14 days, or at least about 15 days.

### Definitions

At various places in the present specification, substituents of compounds of the present disclosure are disclosed in groups or in ranges. It is specifically intended that the present disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

The phrases “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically engineered animal, or a clone.

The term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

The term “acyl,” as used herein, represents a hydrogen or an alkyl group (e.g., a haloalkyl group), as defined herein, that is attached to the parent molecular group through a carbonyl group, as defined herein, and is exemplified by formyl (i.e., a carboxyaldehyde group), acetyl, trifluoroacetyl, propionyl, butanoyl and the like. Example unsubstituted acyl groups include from 1 to 7, from 1 to 11, or from 1 to 21 carbons. In some embodiments, the alkyl group is further substituted with 1, 2, 3, or 4 substituents as described herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, 2-butenyl, and the like. Alkenyls include both cis and trans isomers. Alkenyl groups may be optionally substituted with 1, 2, 3, or 4 substituent groups that are selected, independently, from amino, aryl, cycloalkyl, or heterocyclyl (e.g., heteroaryl), as defined herein, or any of the example alkyl substituent groups described herein.

The term “alkoxy” represents a chemical substituent of formula —OR, where R is a C₁₋₂₀ alkyl group (e.g., C₁₋₆ or C₁₋₁₀ alkyl), unless otherwise specified. Example alkoxy groups include methoxy, ethoxy, propoxy (e.g., n-propoxy and isopropoxy), t-butoxy, and the like. In some embodiments, the alkyl group can be further substituted with 1, 2, 3, or 4 substituent groups as defined herein (e.g., hydroxy or alkoxy).

The term “alkoxyalkyl” represents an alkyl group that is substituted with an alkoxy group. Example unsubstituted alkoxyalkyl groups include between 2 to 40 carbons (e.g., from 2 to 12 or from 2 to 20 carbons, such as C₁₋₆ alkoxy-C₁₋₆ alkyl, C₁₋₁₀ alkoxy-C₁₋₁₀ alkyl, or C₁₋₂₀ alkoxy-C₁₋₂₀ alkyl). In some embodiments, the alkyl and the alkoxy each can be further substituted with 1, 2, 3, or 4 substituent groups as defined herein for the respective group.

The term “alkoxycarbonyl,” as used herein, represents an alkoxy, as defined herein, attached to the parent molecular group through a carbonyl atom (e.g., —C(O)—OR, where R is H or an optionally substituted C₁₋₆, C₁₋₁₀, or C₁₋₂₀ alkyl group). Example unsubstituted alkoxycarbonyl include from 1 to 21 carbons (e.g., from 1 to 11 or from 1 to 7 carbons). In some embodiments, the alkoxy group is further substituted with 1, 2, 3, or 4 substituents as described herein.

The term “alkoxycarbonylalkyl,” as used herein, represents an alkyl group, as defined herein, that is substituted with an alkoxycarbonyl group, as defined herein (e.g., -alkyl-C(O)—OR, where R is an optionally substituted C₁₋₂₀, C₁₋₁₀, or C₁₋₆ alkyl group). Example unsubstituted alkoxycarbonylalkyl include from 3 to 41 carbons (e.g., from 3 to 10, from 3 to 13, from 3 to 17, from 3 to 21, or from 3 to 31 carbons, such as C₁₋₆ alkoxycarbonyl-C₁₋₆ alkyl, C₁₋₁₀ alkoxycarbonyl-C₁₋₁₀ alkyl, or C₁₋₂₀ alkoxycarbonyl-C₁₋₂₀ alkyl). In some embodiments, each alkyl and alkoxy group is further independently substituted with 1, 2, 3, or 4 substituents as described herein (e.g., a hydroxy group).

The term “alkoxycarbonylalkenyl,” as used herein, represents an alkenyl group, as defined herein, that is substituted with an alkoxycarbonyl group, as defined herein (e.g., -alkenyl-C(O)—OR, where R is an optionally substituted C₁₋₂₀, C₁₋₁₀, or C₁₋₆ alkyl group). Example unsubstituted alkoxycarbonylalkenyl include from 4 to 41 carbons (e.g., from 4 to 10, from 4 to 13, from 4 to 17, from 4 to 21, or from 4 to 31 carbons, such as C₁₋₆ alkoxycarbonyl-C₂₋₆ alkenyl, C₁₋₁₀ alkoxycarbonyl-C₂₋₁₀ alkenyl, or C₁₋₂₀ alkoxycarbonyl-C₂₋₂₀ alkenyl). In some embodiments, each alkyl, alkenyl, and alkoxy group is further independently substituted with 1, 2, 3, or 4 substituents as described herein (e.g., a hydroxy group).

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 6 carbon atoms. The alkyl group may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, isopropyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

The term “lower alkyl” means a group having one to six carbons in the chain which chain may be straight or branched. Non-limiting examples of suitable alkyl groups include methyl, ethyl, n-propyl, isopropyl, n-butyl, t-butyl, n-pentyl, and hexyl.

The term “alkylsulfinyl,” as used herein, represents an alkyl group attached to the parent molecular group through an —S(O)— group. Example unsubstituted alkylsulfinyl groups are from 1 to 6, from 1 to 10, or from 1 to 20 carbons. In some embodiments, the alkyl group can be further substituted with 1, 2, 3, or 4 substituent groups as defined herein.

The term “alkylsulfinylalkyl,” as used herein, represents an alkyl group, as defined herein, substituted by an alkylsulfinyl group. Example unsubstituted alkylsulfinylalkyl groups are from 2 to 12, from 2 to 20, or from 2 to 40 carbons. In some embodiments, each alkyl group can be further substituted with 1, 2, 3, or 4 substituent groups as defined herein.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, 1-propynyl, and the like. Alkynyl groups may be optionally substituted with 1, 2, 3, or 4 substituent groups that are selected, independently, from aryl, cycloalkyl, or heterocyclyl (e.g., heteroaryl), as defined herein, or any of the example alkyl substituent groups described herein.

The term “amidine,” as used herein, represents a —C(═NH)NH₂ group.

The term “amino,” as used herein, represents —N(R^{N})₂, wherein each R^{N1} is, independently, H, OH, NO₂, N(R^{N2})₂, SO₂OR^{N2}, SO₂R^{N2}, SOR^{N2}, an N-protecting group, alkyl, alkenyl, alkynyl, alkoxy, aryl, alkaryl, cycloalkyl, alkylcycloalkyl, carboxyalkyl (e.g., optionally substituted with an O-protecting group, such as optionally substituted arylalkoxycarbonyl groups or any described herein), sulfoalkyl, acyl (e.g., acetyl, trifluoroacetyl, or others described herein), alkoxycarbonylalkyl (e.g., optionally substituted with an O-protecting group, such as optionally substituted arylalkoxycarbonyl groups or any described herein), heterocyclyl (e.g., heteroaryl), or alkylheterocyclyl (e.g., alkylheteroaryl), wherein each of these recited R^{N1} groups can be optionally substituted, as defined herein for each group; or two R^{N1} combine to form a heterocyclyl or an N-protecting group, and wherein each R^{N2} is, independently, H, alkyl, or aryl. The amino groups of the disclosure can be an unsubstituted amino (i.e., —NH₂) or a substituted amino (i.e., —N(R′)₂). In a preferred embodiment, amino is —NH₂ or —NHR^{N1}, wherein R^{N1} is, independently, OH, NO₂, NH₂, NR^{N2}₂, SO₂OR^{N2}, SO₂R^{N2}, SOR^{N2}, alkyl, carboxyalkyl, sulfoalkyl, acyl (e.g., acetyl, trifluoroacetyl, or others described herein), alkoxycarbonylalkyl (e.g., t-butoxycarbonylalkyl) or aryl, and each R^{N2} can be H, C₁₋₂₀ alkyl (e.g., C₁₋₆ alkyl), or C₁₋₁₀ aryl.

The term “amino acid,” as described herein, refers to a molecule having a side chain, an amino group, and an acid group (e.g., a carboxy group of —CO₂H or a sulfo group of —SO₃H), wherein the amino acid is attached to the parent molecular group by the side chain, amino group, or acid group (e.g., the side chain). In some embodiments, the amino acid is attached to the parent molecular group by a carbonyl group, where the side chain or amino group is attached to the carbonyl group. Example side chains include an optionally substituted alkyl, aryl, heterocyclyl, alkylaryl, alkylheterocyclyl, aminoalkyl, carbamoylalkyl, and carboxyalkyl. Example amino acids include alanine, arginine, asparagine, aspartic acid, cysteine, glutamic acid, glutamine, glycine, histidine, hydroxynorvaline, isoleucine, leucine, lysine, methionine, norvaline, ornithine, phenylalanine, proline, pyrrolysine, selenocysteine, serine, taurine, threonine, tryptophan, tyrosine, and valine. Amino acid groups may be optionally substituted with one, two, three, or, in the case of amino acid groups of two carbons or more, four substituents independently selected from the group consisting of: (1) C₁₋₆ alkoxy; (2) C₁₋₆ alkylsulfinyl; (3) amino, as defined herein (e.g., unsubstituted amino (i.e., —NH₂) or a substituted amino (i.e., —N(R^{N1})₂, where R^{N1} is as defined for amino); (4) C₆₋₁₀ aryl-C₁₋₆ alkoxy; (5) azido; (6) halo; (7) (C₂₋₉ heterocyclyl)oxy; (8) hydroxy; (9) nitro; (10) oxo (e.g., carboxyaldehyde or acyl); (11) C₁₋₇ spirocyclyl; (12) thioalkoxy; (13) thiol; (14) —CO₂R^{A′}, where R^{A′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alkyl-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (15) —C(O)NR^{B′}R^{C′}, where each of R^{B′} and R^{C′} is, independently, selected from the group consisting of (a) hydrogen, (b) C₁₋₆ alkyl, (c) C₆₋₁₀ aryl, and (d) C₁₋₆ alkyl-C₆₋₁₀ aryl; (16) —SO₂R^{D′}, where R^{D′} is selected from the group consisting of (a) C₁₋₆ alkyl, (b) C₆₋₁₀ aryl, (c) C₁₋₆ alkyl-C₆₋₁₀ aryl, and (d) hydroxy; (17) —SO₂NR^{E′}R^{F′}, where each of R^{E′} and R^{F′} is, independently, selected from the group consisting of (a) hydrogen, (b) C₁₋₆ alkyl, (c) C₆₋₁₀ aryl and (d) C₁₋₆ alkyl-C₆₋₁₀ aryl; (18) —C(O)R^{G′}, where R^{G′} is selected from the group consisting of (a) C₁₋₂₀ alkyl (e.g., C₁₋₆ alkyl), (b) C₂₋₂₀ alkenyl (e.g., C₂₋₆ alkenyl), (c) C₆₋₁₀ aryl, (d) hydrogen, (e) C₁₋₆ alkyl-C₆₋₁₀ aryl, (f) amino-C₁₋₂₀ alkyl, (g) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆alkyl; (19) —NR^{H′}C(O)R^{I′}, wherein R^{H′} is selected from the group consisting of (a1) hydrogen and (b1) C₁₋₆alkyl, and R^{I′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH₂)ₛ₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; (20) —NR^{J′}C(O)OR^{K′}, wherein R^{J′} is selected from the group consisting of (a1) hydrogen and (b1) C₁₋₆ alkyl, and R^{K′} is selected from the group consisting of (a2) C₁₋₂₀ alkyl (e.g., C₁₋₆ alkyl), (b2) C₂₋₂₀ alkenyl (e.g., C₂₋₆ alkenyl), (c2) C₆₋₁₀ aryl, (d2) hydrogen, (e2) C₁₋₆ alkyl-C₆₋₁₀ aryl, (f2) amino-C₁₋₂₀ alkyl, (g2) polyethylene glycol of —(CH₂)ₛ₂(OCH₂CH₂)ₛ₁(CH₂)ₛ₃OR′, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and R′ is H or C₁₋₂₀ alkyl, and (h2) amino-polyethylene glycol of —NR^{N1}(CH)₂(CH₂CH₂O)ₛ₁(CH₂)ₛ₃NR^{N1}, wherein s1 is an integer from 1 to 10 (e.g., from 1 to 6 or from 1 to 4), each of s2 and s3, independently, is an integer from 0 to 10 (e.g., from 0 to 4, from 0 to 6, from 1 to 4, from 1 to 6, or from 1 to 10), and each R^{N1} is, independently, hydrogen or optionally substituted C₁₋₆ alkyl; and (21) amidine. In some embodiments, each of these groups can be further substituted as described herein.

The term “aminoalkyl,” as used herein, represents an alkyl group, as defined herein, substituted by an amino group, as defined herein. The alkyl and amino each can be further substituted with 1, 2, 3, or 4 substituent groups as described herein for the respective group (e.g., CO₂R^{A′}, where R^{A′} is selected from the group consisting of (a) C₁₋₆ alkyl, (b) C₆₋₁₀ aryl, (c) hydrogen, and (d) C₁₋₆ alkyl-C₆₋₁₀ aryl, e.g., carboxy, and/or an N-protecting group).

The term “aminoalkenyl,” as used herein, represents an alkenyl group, as defined herein, substituted by an amino group, as defined herein. The alkenyl and amino each can be further substituted with 1, 2, 3, or 4 substituent groups as described herein for the respective group (e.g., CO₂R^{A′}, where R^{A′} is selected from the group consisting of (a) C₁₋₆ alkyl, (b) C₆₋₁₀ aryl, (c) hydrogen, and (d) C₁₋₆ alkyl-C₆₋₁₀ aryl, e.g., carboxy, and/or an N-protecting group).

The term “anionic lipid” means a lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The terms “include,” “have,” or the like is used in the description or the claims, such term is intended to be inclusive in a manner similar to the term “comprise” as “comprise” is interpreted when employed as a transitional word in a claim.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

The term “biocompatible” means compatible with living cells, tissues, organs or systems posing little to no risk of injury, toxicity or rejection by the immune system.

The term “biodegradable” means capable of being broken down into innocuous products by the action of living things.

The phrase “biologically active” refers to a characteristic of any substance that has activity in a biological system and/or organism. For instance, a substance that, when administered to an organism, has a biological effect on that organism, is considered to be biologically active. In particular embodiments, a polynucleotide of the present disclosure may be considered biologically active if even a portion of the polynucleotide is biologically active or mimics an activity considered biologically relevant.

The terms “carbocyclic” and “carbocyclyl,” as used herein, refer to an optionally substituted C₃₋₁₂ monocyclic, bicyclic, or tricyclic structure in which the rings, which may be aromatic or non-aromatic, are formed by carbon atoms. Carbocyclic structures include cycloalkyl, cycloalkenyl, and aryl groups.

The term “carbamoyl,” as used herein, represents —C(O)—N(R^{N1})₂, where the meaning of each R^{N1} is found in the definition of “amino” provided herein.

The term “carbamoylalkyl,” as used herein, represents an alkyl group, as defined herein, substituted by a carbamoyl group, as defined herein. The alkyl group can be further substituted with 1, 2, 3, or 4 substituent groups as described herein.

The term “carbamyl,” as used herein, refers to a carbamate group having the structure —NR^{N1}C(═O)OR or —OC(═O)N(R^{N1})₂, where the meaning of each R^{N1} is found in the definition of “amino” provided herein, and R is alkyl, cycloalkyl, alkylcycloalkyl, aryl, alkylaryl, heterocyclyl (e.g., heteroaryl), or alkylheterocyclyl (e.g., alkylheteroaryl), as defined herein.

The term “carbonyl,” as used herein, represents a C(O) group, which can also be represented as C═O.

The term “carboxyaldehyde” represents an acyl group having the structure —C(O)H.

The term “carboxy,” as used herein, means —CO₂H.

The term “cationic lipid” means amphiphilic lipids and salts thereof having a positive, hydrophilic head group; one, two, three, or more hydrophobic fatty acid or fatty alkyl chains; and a connector between these two domains. An ionizable or protonatable cationic lipid is typically protonated (i.e., positively charged) at a pH below its pKa and is substantially neutral at a pH above the pKa. Preferred ionizable cationic lipids are those having a pKa that is less than physiological pH, which is typically about 7.4. The cationic lipids of the disclosure may also be termed titratable cationic lipids. The cationic lipids can be an “amino lipid” having a protonatable tertiary amine (e.g., pH-titratable) head group. Some amino exemplary amino lipid can include C18 alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DLinDMA, DLenDMA, 7-DLenDMA, DLin-K-DMA, DLin-K-C2-DMA (also known as DLin-C2K-DMA, XTC2, and C2K), DLin-K-C3-DM A, DLin-K-C4-DMA, DLen-C2K-DMA, y-DLen-C2K-DMA, DLin-M-C2-DMA (also known as MC2), DLin-M-C3-DMA (also known as MC3) and (DLin-MP-DMA)(also known as 1-Bl 1).

The term “comprising” is intended to be open and permits but does not require the inclusion of additional elements or steps. When the term “comprising” is used herein, the term “consisting of” is thus also encompassed and disclosed.

The term “composition” means a product comprising the specified ingredients in the specified amounts, as well as any product that results, directly or indirectly, from combination of the specified ingredients in the specified amounts.

The term “in combination with” means the administration of a lipid formulated mRNA of the present disclosure with other medicaments in the methods of treatment of this disclosure, means-that the lipid formulated mRNA of the present disclosure and the other medicaments are administered sequentially or concurrently in separate dosage forms, or are administered concurrently in the same dosage form.

The term “commercially available chemicals” and the chemicals used in the Examples set forth herein may be obtained from standard commercial sources, where such sources include, for example, Acros Organics (Pittsburgh, Pa.), Sigma-Adrich Chemical (Milwaukee, Wis.), Avocado Research (Lancashire, U.K.), Bionet (Cornwall, U.K.), Boron Molecular (Research Triangle Park, N.C.), Combi-Blocks (San Diego, Calif.), Eastman Organic Chemicals, Eastman Kodak Company (Rochester, N.Y.), Fisher Scientific Co. (Pittsburgh, Pa.), Frontier Scientific (Logan, Utah), ICN Biomedicals, Inc. (Costa Mesa, Calif.), Lancaster Synthesis (Windham, N.H.), Maybridge Chemical Co. (Cornwall, U.K.), Pierce Chemical Co. (Rockford, Ill.), Riedel de Haen (Hannover, Germany), Spectrum Quality Product, Inc. (New Brunswick, N.J.), TCI America (Portland, Oreg.), and Wako Chemicals USA, Inc. (Richmond, Va.).

The phrase “compounds described in the chemical literature” may be identified through reference books and databases directed to chemical compounds and chemical reactions, as known to one of ordinary skill in the art. Suitable reference books and treatise that detail the synthesis of reactants useful in the preparation of compounds disclosed herein, or provide references to articles that describe the preparation of compounds disclosed herein, include for example, “Synthetic Organic Chemistry”, John Wiley and Sons, Inc. New York; S. R. Sandler et al, “Organic Functional Group Preparations,” 2nd Ed., Academic Press, New York, 1983; H. O. House, “Modern Synthetic Reactions,” 2nd Ed., W. A. Benjamin, Inc. Menlo Park, Calif., 1972; T. L. Glichrist, “Heterocyclic Chemistry,” 2nd Ed. John Wiley and Sons, New York, 1992; J. March, “Advanced Organic Chemistry: reactions, Mechanisms and Structure,” 5th Ed., Wiley Interscience, New York, 2001; Specific and analogous reactants may also be identified through the indices of known chemicals prepared by the Chemical Abstract Service of the American Chemical Society, which are available in most public and university libraries, as well as through online databases (the American Chemical Society, Washington, D.C. may be contacted for more details). Chemicals that are known but not commercially available in catalogs may be prepared by custom chemical synthesis houses, where many of the standard chemical supply houses (such as those listed above) provide custom synthesis services.

The term “cycloalkyl,” as used herein represents a monovalent saturated or unsaturated non-aromatic cyclic hydrocarbon group from three to eight carbons, unless otherwise specified, and is exemplified by cyclopropyl, cyclobutyl, cyclopentyl, cyclohexyl, cycloheptyl, bicycle heptyl, and the like. When the cycloalkyl group includes one carbon-carbon double bond, the cycloalkyl group can be referred to as a “cycloalkenyl” group. Exemplary cycloalkenyl groups include cyclopentenyl, cyclohexenyl, and the like. The cycloalkyl groups of this disclosure can be optionally substituted with: (1) C₁₋₇ acyl (e.g., carboxyaldehyde); (2) C₁₋₂₀ alkyl (e.g., C₁₋₆ alkyl, C₁₋₆ alkoxy-C₁₋₆ alkyl, C₁₋₆ alkylsulfinyl-C₁₋₆ alkyl, amino-C₁₋₆ alkyl, azido-C₁₋₆ alkyl, (carboxyaldehyde)-C₁₋₆ alkyl, halo-C₁₋₆ alkyl (e.g., perfluoroalkyl), hydroxy-C₁₋₆ alkyl, nitro-C₁₋₆ alkyl, or C₁₋₆ thioalkoxy-C₁₋₆ alkyl); (3) C₁₂ alkoxy (e.g., C₁₋₆ alkoxy, such as perfluoroalkoxy); (4) C₁₋₆ alkylsulfinyl; (5) C₆₋₁₀ aryl; (6) amino; (7) C₁₋₆ alkyl-C₆₋₁₀ aryl; (8) azido; (9) C₃₋₈ cycloalkyl; (10) C₁₋₆ alkyl-C₃₋₈ cycloalkyl; (11) halo; (12) C₁₋₁₂ heterocyclyl (e.g., C₁₋₁₂ heteroaryl); (13) (C₁₋₁₂ heterocyclyl)oxy; (14) hydroxy; (15) nitro; (16) C₁₋₂₀ thioalkoxy (e.g., C₁₋₆ thioalkoxy); (17) —(CH₂)_{q}CO₂R^{A′}, where q is an integer from zero to four, and R^{A′} is selected from the group consisting of (a) C₁₋₆ alkyl, (b) C₆₋₁₀ aryl, (c) hydrogen, and (d) C₁₋₆ alkyl-C₆₋₁₀ aryl; (18) —(CH₂)_{q}CONR^{B′}R^{C′}, where q is an integer from zero to four and where R^{B′} and R^{C′} are independently selected from the group consisting of (a) hydrogen, (b) C₆₋₁₀ alkyl, (c) C₆₋₁₀ aryl, and (d) C₁₋₆ alkyl-C₆₋₁₀ aryl; (19) —(CH₂)_{q}SO₂R^{D′}, where q is an integer from zero to four and where R^{D′} is selected from the group consisting of (a) C₆₋₁₀ alkyl, (b) C₆₋₁₀ aryl, and (c) C₁₋₆ alkyl-C₆₋₁₀ aryl; (20) —(CH₂)_{q}SO₂NR^{E′}R^{F′}, where q is an integer from zero to four and where each of R^{E′} and R^{F′} is, independently, selected from the group consisting of (a) hydrogen, (b) C₆₋₁₀ alkyl, (c) C₆₋₁₀ aryl, and (d) C₁₋₆ alkyl-C₁₋₁₀ aryl; (21) thiol; (22) C₆₋₁₀ aryloxy; (23) C₃₋₈ cycloalkoxy; (24) C₆₋₁₀ aryl-C₁₋₆ alkoxy; (25) C₁₋₆ alkyl-C₁₋₁₂ heterocyclyl (e.g., C₁₋₆ alkyl-C₁₋₁₂ heteroaryl); (26) oxo; (27) C₂₋₂₀ alkenyl; and (28) C₂₋₂₀ alkynyl. In some embodiments, each of these groups can be further substituted as described herein. For example, the alkyl group of a C₁-alkaryl or a C₁-alkylheterocyclyl can be further substituted with an oxo group to afford the respective aryloyl and (heterocyclyl)oyl substituent group.

The term “diastereomer,” as used herein means stereoisomers that are not mirror images of one another and are non-superimposable on one another.

The term “diacylglycerol” or “DAG” includes a compound having 2 fatty acyl chains, R¹ and R², both of which have independently between 2 and 30 carbons bonded to the 1- and 2-position of glycerol by ester linkages. The acyl groups can be saturated or have varying degrees of unsaturation. Suitable acyl groups include, but are not limited to, lauroyl (C12), myristoyl (C14), palmitoyl (C16), stearoyl (C18), and icosoyl (C20). In preferred embodiments, R¹ and R² are the same, i.e., R¹ and R² are both myristoyl (i.e., dimyristoyl), R¹ and R² are both stearoyl (i.e., distearoyl).

The term “dialkyloxypropyl” or “DAA” includes a compound having 2 alkyl chains, R and R′, both of which have independently between 2 and 30 carbons. The alkyl groups can be saturated or have varying degrees of unsaturation.

The term “effective amount” of an agent, as used herein, is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

The term “enantiomer,” as used herein, means each individual optically active form of a compound of the disclosure, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “fully encapsulated” means that the nucleic acid (e.g., mRNA) in the nucleic acid-lipid particle is not significantly degraded after exposure to serum or a nuclease assay that would significantly degrade free RNA. When fully encapsulated, preferably less than 25% of the nucleic acid in the particle is degraded in a treatment that would normally degrade 100% of free nucleic acid, more preferably less than 10%, and most preferably less than 5% of the nucleic acid in the particle is degraded. “Fully encapsulated” also means that the nucleic acid-lipid particles do not rapidly decompose into their component parts upon in vivo administration.

The terms “halo” and “Halogen”, as used herein, represents a halogen selected from bromine, chlorine, iodine, or fluorine.

The term “haloalkyl,” as used herein, represents an alkyl group, as defined herein, substituted by a halogen group (i.e., F, Cl, Br, or I). A haloalkyl may be substituted with one, two, three, or, in the case of alkyl groups of two carbons or more, four halogens. Haloalkyl groups include perfluoroalkyls (e.g., —CF₃), —CHF₂, —CH₂F, —CCl₃, —CH₂CH₂Br, —CH₂CH(CH₂CH₂Br)CH₃, and —CHICH₃. In some embodiments, the haloalkyl group can be further substituted with 1, 2, 3, or 4 substituent groups as described herein for alkyl groups.

The term “heteroalkyl,” as used herein, refers to an alkyl group, as defined herein, in which one or two of the constituent carbon atoms have each been replaced by nitrogen, oxygen, or sulfur. In some embodiments, the heteroalkyl group can be further substituted with 1, 2, 3, or 4 substituent groups as described herein for alkyl groups.

The term “hydrocarbon,” as used herein, represents a group consisting only of carbon and hydrogen atoms.

The term “hydroxy,” as used herein, represents an —OH group. In some embodiments, the hydroxy group can be substituted with 1, 2, 3, or 4 substituent groups (e.g., O-protecting groups) as defined herein for an alkyl.

The term “hydroxyalkenyl,” as used herein, represents an alkenyl group, as defined herein, substituted by one to three hydroxy groups, with the proviso that no more than one hydroxy group may be attached to a single carbon atom of the alkyl group, and is exemplified by dihydroxypropenyl, hydroxyisopentenyl, and the like. In some embodiments, the hydroxyalkenyl group can be substituted with 1, 2, 3, or 4 substituent groups (e.g., O-protecting groups) as defined herein for an alkyl.

The term “hydroxyalkyl,” as used herein, represents an alkyl group, as defined herein, substituted by one to three hydroxy groups, with the proviso that no more than one hydroxy group may be attached to a single carbon atom of the alkyl group, and is exemplified by hydroxymethyl, dihydroxypropyl, and the like. In some embodiments, the hydroxyalkyl group can be substituted with 1, 2, 3, or 4 substituent groups (e.g., O-protecting groups) as defined herein for an alkyl.

The term “hydrate” means a solvate wherein the solvent molecule is H₂O.

The term “isomer,” as used herein, means any tautomer, stereoisomer, enantiomer, or diastereomer of any compound of the disclosure. It is recognized that the compounds of the disclosure can have one or more chiral centers and/or double bonds and, therefore, exist as stereoisomers, such as double-bond isomers (i.e., geometric E/Z isomers) or diastereomers (e.g., enantiomers (i.e., (+) or (−)) or cis/trans isomers). According to the disclosure, the chemical structures depicted herein, and therefore the compounds of the disclosure, encompass all of the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

The term “nitro,” as used herein, represents an —NO₂ group.

The term “nucleic acid” means deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides.

Examples of such analogs include, without limitation, phosphorothioates, phosphoramidates, methyl phosphonates, chiral-methyl phosphonates, 2′-O-methyl ribonucleotides, peptide-nucleic acids (PNAs).

The term “oxo” as used herein, represents ═O.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers and/or conformers of the basic molecular structure. Some compounds of the present disclosure may exist in different tautomeric forms, all of the latter being included within the scope of the present disclosure.

The term “sulfonyl,” as used herein, represents an —S(O)₂— group.

The term “compound,” is meant to include all stereoisomers, geometric isomers, tautomers, and isotopes of the structures depicted.

The term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

The term “cyclic” refers to the presence of a continuous loop. Cyclic molecules need not be circular, only joined to form an unbroken chain of subunits. Cyclic molecules such as the mRNA of the present disclosure may be single units or multimers or comprise one or more components of a complex or higher order structure.

The term “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

The term “delivery” refers to the act or manner of delivering a compound, substance, entity, moiety, cargo or payload.

The term “delivery agent” refers to any substance which facilitates, at least in part, the in vivo delivery of a polynucleotide to targeted cells.

The term “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

The term “feature” refers to a characteristic, a property, or a distinctive element.

The term “fragment,” as used herein, refers to a portion. For example, fragments of proteins may comprise polypeptides obtained by digesting full-length protein isolated from cultured cells.

The term “functional” biological molecule is a biological molecule in a form in which it exhibits a property and/or activity by which it is characterized.

The term “hydrophobic lipids” means compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacyloxy-3-aminopropane, and 1,2-dialkyl-3-aminopropane.

The term “lipid” means an organic compound that comprises an ester of fatty acid and is characterized by being insoluble in water, but soluble in many organic solvents. Lipids are usually divided into at least three classes: (1) “simple lipids,” which include fats and oils as well as waxes; (2) “compound lipids,” which include phospholipids and glycolipids; and (3) “derived lipids” such as steroids.

The term “lipid delivery vehicle” means a lipid formulation that can be used to deliver a therapeutic nucleic acid (e.g., mRNA) to a target site of interest (e.g., cell, tissue, organ, and the like). The lipid delivery vehicle can be a nucleic acid-lipid particle, which can be formed from a cationic lipid, a non-cationic lipid (e.g., a phospholipid), a conjugated lipid that prevents aggregation of the particle (e.g., a PEG-lipid), and optionally cholesterol. Typically, the therapeutic nucleic acid (e.g., mRNA) may be encapsulated in the lipid portion of the particle, thereby protecting it from enzymatic degradation.

The term “lipid encapsulated” means a lipid particle that provides a therapeutic nucleic acid such as an mRNA with full encapsulation, partial encapsulation, or both. In a preferred embodiment, the nucleic acid (e.g., mRNA) is fully encapsulated in the lipid particle.

The term “amphipathic lipid” or “amphiphilic lipid” means the material in which the hydrophobic portion of the lipid material orients into a hydrophobic phase, while the hydrophilic portion orients toward the aqueous phase. Hydrophilic characteristics derive from the presence of polar or charged groups such as carbohydrates, phosphate, carboxylic, sulfato, amino, sulfhydryl, nitro, hydroxyl, and other like groups. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids.

The term “linker” or “linking moiety” refers to a group of atoms, e.g., 10-100 atoms, and can be comprised of the atoms or groups such as, but not limited to, carbon, amino, alkylamino, oxygen, sulfur, sulfoxide, sulfonyl, carbonyl, and imine. The linker may be of sufficient length as to not interfere with incorporation into an amino acid sequence. Examples of chemical groups that can be incorporated into the linker include, but are not limited to, alkyl, alkenyl, alkynyl, amido, amino, ether, thioether, ester, alkyl, heteroalkyl, aryl, or heterocyclyl, each of which can be optionally substituted, as described herein. Examples of linkers include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers, Other examples include, but are not limited to, cleavable moieties within the linker, such as, for example, a disulfide bond (—S—S—) or an azo bond (—N═N—), which can be cleaved using a reducing agent or photolysis. Non-limiting examples of a selectively cleavable bond include an amido bond, which can be cleaved for example by the use of tris(2-carboxyethyl)phosphine (TCEP), or other reducing agents, and/or photolysis, as well as an ester bond, which can be cleaved for example by acidic or basic hydrolysis.

The term “mammal” means a human or other mammal or means a human being.

The term “messenger RNA” (mRNA) refers to any polynucleotide which encodes a protein or polypeptide of interest and which is capable of being translated to produce the encoded protein or polypeptide of interest in vitro, in vivo, in situ or ex vivo.

The term “modified” refers to a changed state or structure of a molecule of the disclosure. Molecules may be modified in many ways including chemically, structurally, and functionally. In one embodiment, nucleic acid active ingredients are modified by the introduction of non-natural nucleosides and/or nucleotides, e.g., as it relates to the natural ribonucleotides A, U, G, and C. Noncanonical nucleotides such as the cap structures are not considered “modified” although they may differ from the chemical structure of the A, C, G, U ribonucleotides.

The term “naturally occurring” means existing in nature without artificial aid.

The term “nonhuman vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

The term “nucleotide” means natural bases (standard) and modified bases well known in the art. Such bases are generally located at the 1′ position of a nucleotide sugar moiety.

Nucleotides generally comprise a base, sugar, and a phosphate group. The nucleotides can be unmodified or modified at the sugar, phosphate, and/or base moiety, (also referred to interchangeably as nucleotide analogs, modified nucleotides, non-natural nucleotides, non-standard nucleotides and other; see, for example, Usman and McSwiggen, supra; Eckstein, et al., International PCT Publication No. WO 92/07065; Usman, et al., International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include: inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine, thymine and uracil at 1′ position or their equivalents.

The phrase “operably linked” refers to a functional connection between two or more molecules, constructs, transcripts, entities, moieties or the like.

The term “patient” refers to a subject who may seek or be in need of treatment, requires treatment, is receiving treatment, will receive treatment, or a subject who is under care by a trained professional for a particular disease or condition.

The phrase “optionally substituted X” (e.g., optionally substituted alkyl) is intended to be equivalent to “X, wherein X is optionally substituted” (e.g., “alkyl, wherein said alkyl is optionally substituted”). It is not intended to mean that the feature “X” (e.g. alkyl) per se is optional.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The phrase “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

The term “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

The term “pharmaceutically acceptable solvate,” as used herein, means a compound of the disclosure wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “physicochemical” means of or relating to a physical and/or chemical property.

The term “phosphate” is used in its ordinary sense as understood by those skilled in the art and includes its protonated forms, for example

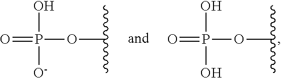 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The term “phosphorothioate” refers to a compound of the general formula

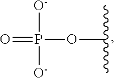

its protonated forms, for example,

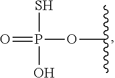 [SMILES: *OP(=O)(O)S]

and its tautomers such as

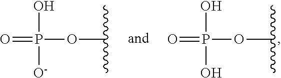 [SMILES: *OP(=O)([O-])O ; *OP(=O)(O)O]

The term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

The term “proteins of interest” or “desired proteins” include those provided herein and fragments, mutants, variants, and alterations thereof.

The terms “purify,” “purified,” “purification” means to make substantially pure or clear from unwanted components, material defilement, admixture or imperfection.

The term “RNA” means a molecule comprising at least one ribonucleotide residue. By “ribonucleotide” is meant a nucleotide with a hydroxyl group at the 2′ position of a β-D-ribo-furanose moiety. The terms includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution, and/or alteration of one or more nucleotides. Such alterations can include addition of non-nucleotide material, such as to the end(s) of an interfering RNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogs or analogs of naturally-occurring RNA. As used herein, the terms “ribonucleic acid” and “RNA” refer to a molecule containing at least one ribonucleotide residue, including siRNA, antisense RNA, single stranded RNA, microRNA, mRNA, noncoding RNA, and multivalent RNA.

The term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

The terms “significant” or “significantly” are used synonymously with the term “substantially.”

The phrase “single unit dose” is a dose of any therapeutic administered in one dose/at one time/single route/single point of contact, i.e., single administration event.

The term “siRNA” or small interfering RNA, sometimes known as short interfering RNA or silencing RNA, refers to a class of double-stranded RNA non-coding RNA molecules, typically 18-27 base pairs in length, similar to miRNA, and operating within the RNA interference (RNAi) pathway. It interferes with the expression of specific genes with complementary nucleotide sequences by degrading mRNA after transcription, thereby preventing translation.

The term “solvate” means a physical association of a compound of this disclosure with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like.

The term “split dose” is the division of single unit dose or total daily dose into two or more doses.

The term “stable” refers to a compound that is sufficiently robust to survive isolation to a useful degree of purity from a reaction mixture, and preferably capable of formulation into an efficacious therapeutic agent.

The terms “stabilize”, “stabilized,” “stabilized region” means to make or become stable.

The term “substituted” means substitution with specified groups other than hydrogen, or with one or more groups, moieties, or radicals which can be the same or different, with each, for example, being independently selected.

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The phrase “Substantially equal” relates to time differences between doses, the term means plus/minus 2%.

The phrase “substantially simultaneously” relates to plurality of doses, the term means within 2 seconds.

The phrase “suffering from” relates to an individual who is “suffering from” a disease, disorder, and/or condition has been diagnosed with or displays one or more symptoms of a disease, disorder, and/or condition.

The phrase “susceptible to” relates to an individual who is “susceptible to” a disease, disorder, and/or condition has not been diagnosed with and/or may not exhibit symptoms of the disease, disorder, and/or condition but harbors a propensity to develop a disease or its symptoms.

In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

The term “synthetic” means produced, prepared, and/or manufactured by the hand of man. Synthesis of polynucleotides or polypeptides or other molecules of the present disclosure may be chemical or enzymatic.

The term “targeted cells” refers to any one or more cells of interest. The cells may be found in vitro, in vivo, in situ or in the tissue or organ of an organism. The organism may be an animal, preferably a mammal, more preferably a human and most preferably a patient.

The term “therapeutic agent” refers to any agent that, when administered to a subject, has a therapeutic, diagnostic, and/or prophylactic effect and/or elicits a desired biological and/or pharmacological effect.

The term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

The term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

The term “total daily dose” is an amount given or prescribed in 24-hour period. It may be administered as a single unit dose.

The term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The term “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

The term “half-life” is the time required for a quantity such as nucleic acid or protein concentration or activity to fall to half of its value as measured at the beginning of a time period.

The term “in vitro” refers to events that occur in an artificial environment, e.g., in a test tube or reaction vessel, in cell culture, in a Petri dish, etc., rather than within an organism (e.g., animal, plant, or microbe).

The term “in vivo” refers to events that occur within an organism (e.g., animal, plant, or microbe or cell or tissue thereof).

The term “monomer” refers to a single unit, e.g., a single nucleic acid, which may be joined with another molecule of the same or different type to form an oligomer. In some embodiments, a monomer may be an unlocked nucleic acid, i.e., a UNA monomer.

The term “neutral lipid” means a lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” means an amphipathic lipid or a neutral lipid or anionic lipid and is described herein.

The terms “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

The term “translatable” may be used interchangeably with the term “expressible” and refers to the ability of polynucleotide, or a portion thereof, to be converted to a polypeptide by a host cell. As is understood in the art, translation is the process in which ribosomes in a cell's cytoplasm create polypeptides. In translation, messenger RNA (mRNA) is decoded by tRNAs in a ribosome complex to produce a specific amino acid chain, or polypeptide. Furthermore, the term “translatable” when used in this specification in reference to an oligomer, means that at least a portion of the oligomer, e.g., the coding region of an oligomer sequence (also known as the coding sequence or CDS), is capable of being converted to a protein or a fragment thereof.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

The term “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

While this disclosure has been described in relation to certain embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications, and equivalents. In particular, this disclosure includes any combination of the features, terms, or elements of the various illustrative components and examples.

### EXAMPLES

The present disclosure is further described in the following examples, which do not limit the scope of the disclosure described in the claims.

### Example 1: Synthesis of Peptides and Example Peptide-Lipid Conjugate Peptide Synthesis

Generally, peptides were synthesized on a peptide synthesizer using standard N-(9-Fluorenylmethoxycarbonyloxy) (Fmoc) protecting group (B) chemistry and purified with HPLC on a C18 column. Briefly, Peptide synthesis was done on a Prelude X peptide synthesizer (Protein Technologies, Inc.; Tucson, Ariz.) in a linear fashion following Solid Phase Peptide Synthesis protocol using, Fmoc protected amino acids, Fmoc-Glu(OtBu)-OH, Fmoc-Pro-OH, Fmoc-Ser(tBu)-OH, Fmoc-Thr(tBu)-OH, Fmoc-Ser(Me)-OH, FmocThr(Me)-OH as building block reagents and N,N-dimethylformamide, acetonitrile, diethyl ether and dichloromethane as solvents of choice for various steps. First, Fmoc-Pro-OH was loaded on to 2-ClTrityl resin (0.6 eq. relative to the resin, 4 eq. N,N-Diisopropylethylamine (DIEA)). Then, Fmoc was deprotected using 20% piperidine (2× for 5 min.). This was followed by coupling 7.5 eq. of desired Fmoc-AA, HCT as an activator and 15 eq. NNM as a base. A double coupling approach for 25 min. and 20 min. was used to ensure complete coupling. The Fmoc deprotection and double coupling steps were repeated for all amino acids and until desired peptide is synthesized. Each peptide on the resin was dried and cleaved from the resin using a cocktail of 90% TFA, 5% thioanisole, 2.5% H2O, 1.5% ethanedithiol and 1% phenol by volume for 2 hours at ambient temperature. Further, each peptide was purified on reverse phase high performance liquid chromatography (RP-HPLC) using a Jupiter 10 u Proteo column of 250×21.2 mm size (Phenomenex, Torrance, Calif.). A Mobile phase of solvent A of 0.1% TFA in H2O and solvent B of 0.1% TFA in 80% Acetonitrile was used with gradient of mobile phase B from 18% to 38% within 20 minutes. A flow rate of 15 ml/min and a UV detection wavelength of 214 nm were used. Major product-containing fractions were analyzed, pooled and solvent removed to get pure peptide.

To form a peptide-lipid conjugate from the peptides, each peptide was coupled at the N-terminal amine with (R)-2,3-bis(tetradecanoyloxy)propyl (2,5-dioxopyrrolidin-1-yl) succinate (Compound 3 below) to get the final DMG-SA-peptide conjugate. Briefly, the linear peptide obtained above and DMG-SA-NHS (N-hydroxy succinimide) (eq 1:1.2) are dissolved in DMF (dimethyl formamide) in the presence of 2 eq. DIEA overnight. The product formed (DMG-SA-peptide) was then precipitated in cold ether. These conjugates were further purified on a C8 column and lyophilized without any additional additives at −80° C. on a Labconco lyophilizer (Kansas City, Mo.) to get the pure products as white powders. The final yield ranged from about 60-80%. This coupling reaction and conjugated lipid described in this example were chosen to provide a proof of concept for the conjugated peptides of the disclosure, and a person of ordinary skill in the art will recognize other suitable coupling reactions and lipids known in the art for conjugation with the peptides of the disclosure. In addition, methods for coupling the peptide at its C-terminus or at one of the amino acid side chains are well known in the art.

The example peptides made in this study are listed in Table 1 below.

TABLE 1
Example Peptides Synthesized

| Peptide | | |
|---|---|---|
| Lipid | | |
| Conjugate | | Molecular |
| Reference | Sequence (In an N-terminal to C-terminal Direction) | Weight |
| Peptide 2 | X-STEPSTEPSTEPSTEP-OH | 2270.13 |
| Peptide 3 | X-STEPSTEPSTEPSTEPSTEP-OH | 2684.55 |
| Peptide 5 | X-S(Me)T(Me)QPS(Me)T(Me)QPS(Me)T(Me)QPS(Me)T(Me)QP-OH | 2378.21 |
| Peptide 6 | X-S(Me)T(Me)QPS(Me)T(Me)QPS(Me)T(Me)QPS(Me)T(Me)QPS(Me)T(Me)QP-OH | 2819.65 |
| Peptide 7 | X-STEPSTEPSTEPSTEP-NH | 2269.14 |
| Peptide 8 | X-S(Me)T(Me)QPS(Me)T(Me)QPS(Me)T(Me)QPS(Me)T(Me)QP-NH₂ | 2377.22 |

X = A conjugated lipid after coupling of Compound 3 of Scheme 1 below
S = Serine
T = Threonine
E = Glutamic Acid
P = Proline, with P-OH representing proline, and P-NH₂ representing prolinamide, which was used to masking a negative charge at the C-terminus.
S(Me) = Methyl Serine
T(Me) = Methyl Threonine
Q = Glutamine

### Synthesis of DMG-Peptide Conjugates

Example peptide-lipid conjugates were made using the peptides described herein conjugated to an example lipid compound (R)-2,3-bis(tetradecanoyloxy)propyl (2,5-dioxopyrrolidin-1-yl) succinate (Group 3) per synthetic Scheme 1 described herein.

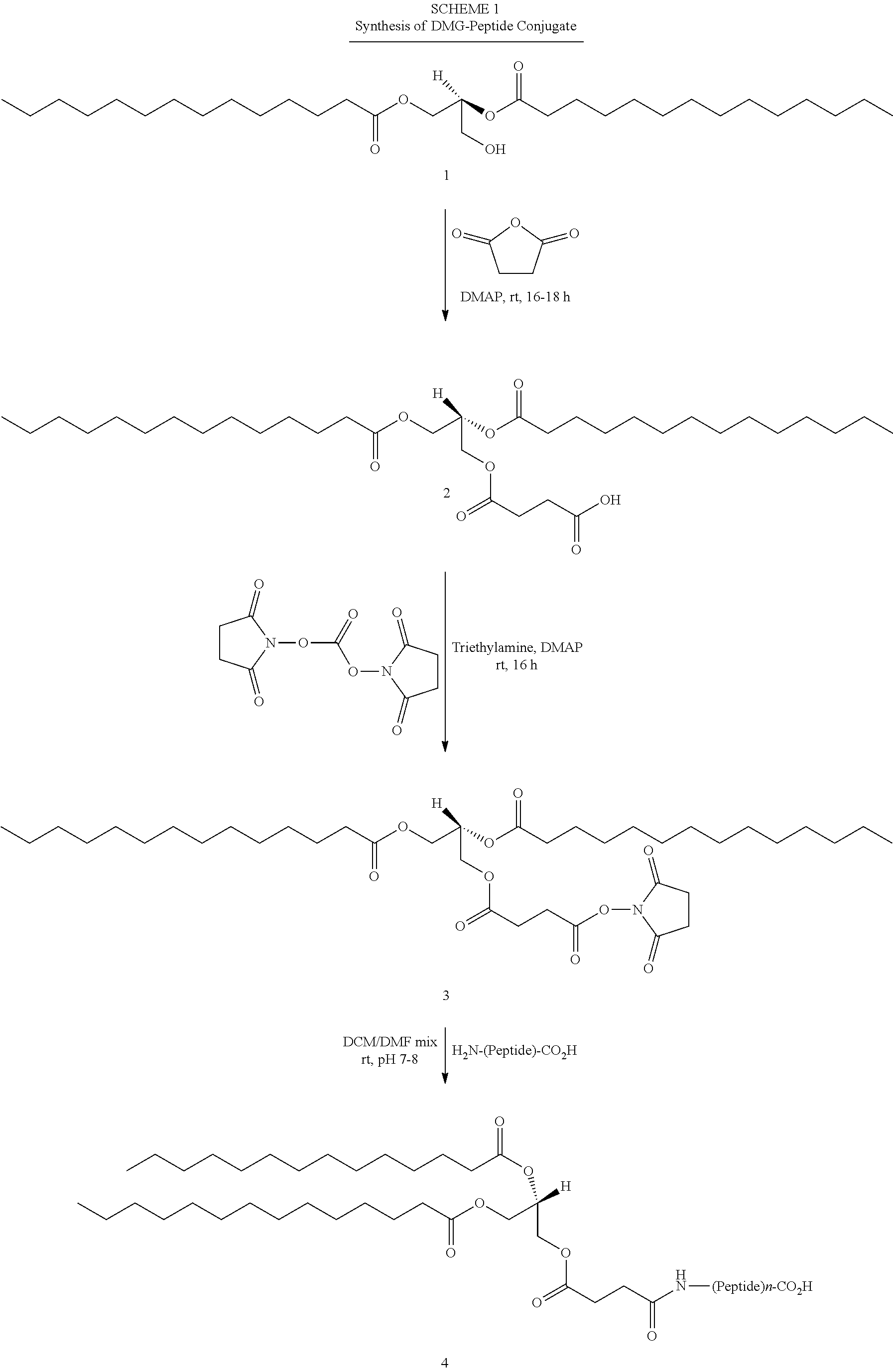

### i (R)-4-(2,3-bis(tetradecanoyloxy)propoxy)-4-oxobutanoic acid (Compound 2 in Scheme 1)

Succinic anhydride (670 mg, 6.6 mmol) and N,N-dimethylaminopyridine (DMAP, 1.0 g, 8.3 mmol) was added to a solution of (S)-3-hydroxypropane-1,2-diyl ditetradecanoate (Compound 1 in Scheme 1, 2.05 g, 4 mmol) in 40 mL of dichloromethane at room temperature. This mixture was stirred at ambient temperature for 16-18 hours. A 1 M aqueous hydrochloric acid aliquot (8.5 mL) was added to quench the reaction. The mixture was diluted with 20 mL water and the organic layer was separated. The aqueous layer was extracted with another 40 mL dichloromethane and the combined organic solution was washed with 1 M aqueous HCl (1×100 mL), dried over anhydrous sodium sulfate, and concentrated on a rotoevaporator under reduced pressure. The resulting semi-solid was dried under high vacuum over phosphorus pentoxide to obtain 2.4 g of product as a white solid. m/z 612.46 (Calculated) M−H 611.7 (Observed).

### ii. (R)-2,3-bis(tetradecanoyloxy)propyl (2,5-dioxopyrrolidin-1-yl) succinate (Compound 3 in Scheme 1)

To a mixture of (R)-4-(2,3-bis(tetradecanoyloxy)propoxy)-4-oxobutanoic acid (8.8 g, 28.7 mmol), triethylamine (2.9 g, 19.6 mmol) and 80 mg DMAP in 160 mL dichloromethane was added succinimidyl carbonate (5.04 g, 19.6 mmol), and the mixture was stirred at room temperature for 16 hours. Two equivalents of glacial acetic acid were added to quench the reaction. The mixture was diluted with another 100 mL DCM and washed with ice-cold water (2×300 mL), followed by brine (1×300 mL). The organic phase was separated, dried (anhydrous sodium sulfate), and solvent was removed under reduced pressure. The residue was purified on a 80 g Teledyne ISCO silica gel column using a gradient of dichloromethane:ethylacetate. Fractions eluted at 10-12% ethyl acetate concentration was pooled and concentrated under reduced pressure to obtain 9 g of product as a while solid. m/z 709.5 (Calculated) M+Na 732.2 (Observed).

### iii. DMG-SA-(Peptide) Peptide Synthesis (Illustrated by Compound 4 of Scheme 1)

Each synthetic peptide as described in this example was coupled at the N-terminal amine with (R)-2,3-bis(tetradecanoyloxy)propyl (2,5-dioxopyrrolidin-1-yl) succinate to get the final DMG-SA-(Peptide) conjugate. These conjugates were further purified on a C8 column and lyophilized to get pure products as white powders.

### Example 2: Protocol for Lipid Nanoparticle Preparation

The peptide-lipid conjugates of the present disclosure were tested in nucleic acid-lipid formulations. Lipid nanoparticles (LNPs) encapsulating FVII siRNA or human erythropoietin (hEPO) mRNA were prepared in accordance with the methods described by Ramaswamy et al. (Proc. Natl. Acad. Sci. USA. 2017 Mar. 7; 114(10):E1941-E1950) by mixing an ethanolic solution of lipids with an aqueous solution of RNA. Briefly, lipid excipients (ionizable lipid, DSPC, Cholesterol and PEG2000-DMG or peptide-lipid conjugate of the disclosure) are dissolved in ethanol at a specific mole ratio. An aqueous solution of the RNA is prepared in citrate buffer between pH 3-4. The lipid mixture is then combined with the RNA solution at a flow rate ratio of 1:3 (V/V) using the Nanoassemblr microfluidic system (Precision NanoSystems, Vancouver, BC, Canada). Nanoparticles thus formed are purified by a tangential flow filtration (TFF) process. The concentration of the resulting formulation is then adjusted to a final target RNA concentration using 100,000 MWCO Amicon Ultra centrifuge tubes (Millipore Sigma) followed by filtration through a 0.2 μm PES sterilizing-grade filter. Post filtration, bulk formulation is aseptically filled into sterile Eppendorf tubes and frozen at −70 f 10° C. Analytical characterization of the lipid nanoparticles includes measurement of particle size and polydispersity using dynamic light scattering (ZEN3600, Malvern Instruments), RNA content and encapsulation efficiency by a fluorometric assay using RiboGreen RNA reagent (Thermo Fisher Scientific).

### Example 3: Protocol for Factor VII Knock Down Evaluation

Lipid formulations comprising a FVII siRNA further described below were evaluated for their knockdown activity using the protocol of this example. In the FVII evaluation, seven to eight week-old, female Balb/C mice were purchased from Charles River Laboratories (Hollister, Calif.). The mice were held in a pathogen-free environment and all procedures involving the mice were performed in accordance with guidelines established by the Institutional Animal Care and Use Committee (IACUC). Lipid nanoparticles containing factor VII siRNA were administered intravenously at a dosing volume of 10 mL/kg and two dose levels (0.03 and 0.01 mg/kg). After 48 h, the mice were anesthetized with isoflurane and blood was collected retro-orbitally into Microtainer® tubes coated with 0.109 M sodium citrate buffer (BD Biosciences, San Diego, Calif.) and processed to plasma. Plasma specimens were tested for factor VII levels immediately or stored at −80° C. for later analysis. Measurement of FVII protein in plasma was determined using the colorimetric Biophen VII assay kit (Aniara Diagnostica, USA). Absorbance was measured at 405 nm and a calibration curve was generated using the serially diluted control plasma to determine levels of factor VII in plasma from treated animals, relative to the saline-treated control animals.

### Example 4: Protocol for hEPO mRNA Expression Evaluation

Lipid formulations comprising a hEPO mRNA below were evaluated for their ability to express hEPO in vivo according to the protocol of this example. All animal experiments were conducted using institutionally-approved protocols (IACUC). In this protocol, female Balb/c mice at least 6-8 weeks of age were purchased from Charles River Laboratory. The mice were intravenously injected with hEPO-LNPs via the tail vein with one of two dose levels of hEPO (0.1 and 0.03 mg/kg). After 6 hr, blood was collected with serum separation tubes, and the serum was isolated by centrifugation. Serum hEPO levels were then measured using an ELISA assay (Human Erythropoietin Quantikine IVD ELISA Kit, R&D Systems, Minneapolis, Md.).

### Example 5: Biodistribution and Immunostaining Protocol

Studies assessing the biodistribution and immunostaining of formulations described herein were conducted per the protocol described in this example. In this protocol, transgenic floxed tdTomato mice were used. These mice were engineered to have a gene encoding tdTomato fluorescent reporter protein but also includes a CRE-based stop cassette (i.e., floxed cassette), which prevents complete transcription of the tdTomato gene in the absence of a protein called CRE recombinase (CRE). The floxed tdTomato mice are further deficient in the CRE gene.

A total of six floxed tdTomato mice were divided into three groups of two mice. The control group was injected with PBS and the two remaining groups were injected with LNP formulations containing CRE-tdTomato mRNA. The LNP formulations included either PEG-DMG or Peptide 7. One mouse from each group received an intravenous (IV) injection and the other mouse revived an intramuscular (IM) injection. The animals were dosed at 1 mg/kg of mRNA and a volume of 10 mL/kg. At 72 h post injection, the mice were euthanized. For mice dosed by IV injection, organs including the liver, spleen, lung, kidney and heart were removed.

For mice dosed by IM injection, the sites of injection were removed, including the left rectus femoris, right rectus femoris, liver and spleen. The organs were fixed in 10% neutral buffered formalin, embedded into paraffin blocks, and cut into 5 μm sections. Each section was stained by using tdTomato antibody and for secondary detection by immunohistochemistry. The sections were then incubated with 1:300 dilutions of biotin-labeled anti-rabbit (ab6801) and stained using streptavidin-horseradish peroxidase (HRP) (20774, Millipore) and 3,3′-diaminobenzidine (DAB) substrate (SK-4100, Vector Laboratories). Confocal immunofluorescence microscopy was used to collect images of the samples.

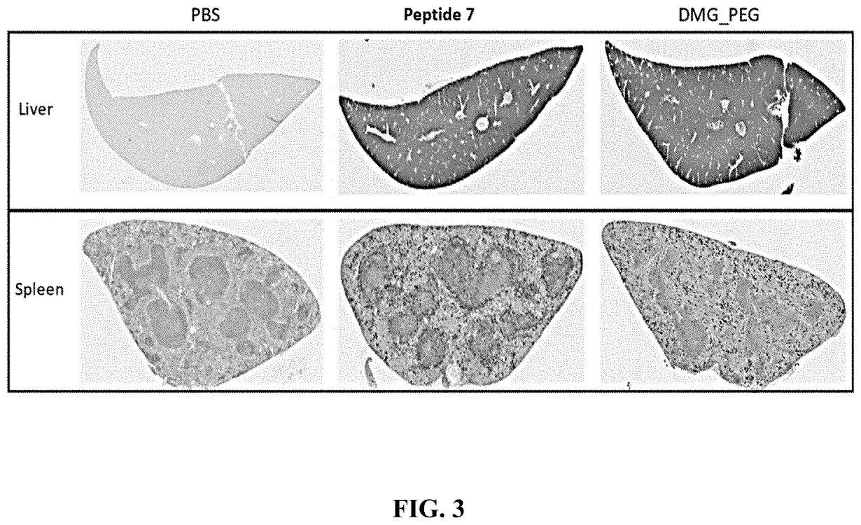

The degree of successful treatment of mice transfected with CRE mRNA-lipid formulations is indicated by expression of the tdTomato proteins as such mice are able to generate a CRE protein that excises out the floxed cassette, allowing the expression of the tdTomato protein. As illustrated in FIG. 3, LNP formulations including the peptides described herein are able to efficiently deliver mRNA to organs in mice.

### Example 6: Example Lipid Nanoparticle Formulations

Lipid nanoparticle formulation encapsulating either a FVII siRNA or hEPO mRNA were prepared as described in the protocol of Example 2 above. These lipid nanoparticle formulations included an ionizable cationic lipid (“Cat”), helper lipid (distearoylphosphatidylcholine, “DSPC”), cholesterol (“Chol”), and either a lipid-peptide conjugate or a PEG-lipid conjugate. The ionizable cationic lipid used in these formulations was selected to provide a common lipid that could serve as a basis for comparison, however a person of skill in the art would recognize that the lipid-peptide conjugates of the disclosure can be combined with any cationic lipid suitable for use in a lipid nanoparticle formulation for the delivery of an active agent such as a nucleic acid. The ionizable cationic lipid used in these formulations has the following structure:

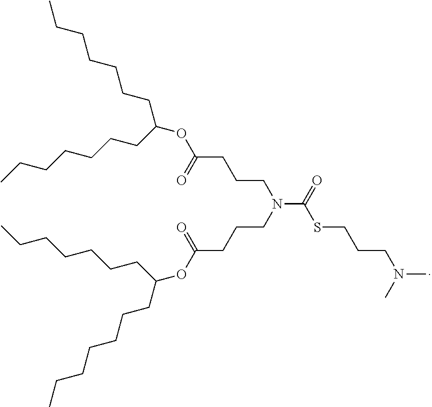 [SMILES: CCCCCCCC(CCCCCCC)OC(=O)CCCN(CCCC(=O)OC(CCCCCCC)CCCCCCC)C(=O)SCCCN(C)C]

The example lipid nanoparticle formulations were prepared and characterized as described in Example 2, the details of each formulation together with the resultant characteristics are provided in Table 2 below. In this table, “N/P” refers to the ratio of cationic amino groups from the ionizable cationic lipid to the anionic phosphate backbone groups of the encapsulated nucleic acid. The results indicate that the peptide-lipid conjugates of the disclosure integrate well into lipid nanoparticle formulations with good particle size, polydispersity, and percent encapsulation of the nucleic acid.

TABLE 2
Example Lipid Nanoparticle Formulations

| | Nucleic | Diameter | | Percent |
|---|---|---|---|---|
| Lipid Composition | Acid | (nm) | Polydispersity | Encapsulation |
| Cat:DSPC:Chol:Peptide 2-DMG | FVII | 81.16 | 0.074 | 99.28 |
| (45:10:44:1), N/P 9 | siRNA | | | |
| Cat:DSPC:Chol:Peptide 3-DMG | FVII | 90.16 | 0.036 | 99.28 |
| (45:10:44:1), N/P 9 | siRNA | | | |
| Cat:DSPC:Chol:Peptide 5-DMG | FVII | 76.04 | 0.1 | 98.86 |
| (45:10:44:1), N/P 9 | siRNA | | | |
| Cat:DSPC:Chol:Peptide 6-DMG | FVII | 79.82 | 0.216 | 99.15 |
| (45:10:44:1), N/P 9 | siRNA | | | |
| Cat:DSPC:Chol:Peptide 7-DMG | FVII | 83.87 | 0.07 | 99.30 |
| (45:10:44:1), N/P 9 | siRNA | | | |
| Cat:DSPC:Chol:Peptide 8-DMG | FVII | 62.96 | 0.096 | 98.95 |
| (45:10:44:1), N/P 9 | siRNA | | | |
| Cat:DSPC:Chol:Peptide 2-DMG | hEPO | 74.01 | 0.127 | 98.9 |
| (40:15:44:1), N/P 9 | mRNA | | | |
| Cat: DSPC: Chol: PEG200-DMG | FVII | 70.71 | 0.3 | 98.8 |
| (40:15:44:1), N/P 9 | siRNA | | | |
| Cat:DSPC:Chol:Peptide 3-DMG | hEPO | 91.31 | 0.207 | 99 |
| (40:15:44:1), N/P 9 | mRNA | | | |
| Cat:DSPC:Chol:Peptide 5-DMG | hEPO | 85.11 | 0.131 | 93.8 |
| (40:15:44:1), N/P 9 | mRNA | | | |
| Cat:DSPC:Chol:Peptide 6-DMG | hEPO | 65.95 | 0.185 | 92.2 |
| (40:15:44:1), N/P 9 | mRNA | | | |
| Cat:DSPC:Chol:Peptide 7-DMG | hEPO | 73.21 | 0.15 | 95.2 |
| (40:15:44:1), N/P 9 | mRNA | | | |
| Cat:DSPC:Chol:Peptide 8-DMG | hEPO | 81.02 | 0.136 | 98.7 |
| (40:15:44:1), N/P 9 | mRNA | | | |
| Cat: DSPC: Chol: PEG200-DMG | hEPO | 77.77 | 0.26 | 96.3 |
| (40:15:44:1), N/P 9 | mRNA | | | |

### Example 7: EPO Expression In Vivo

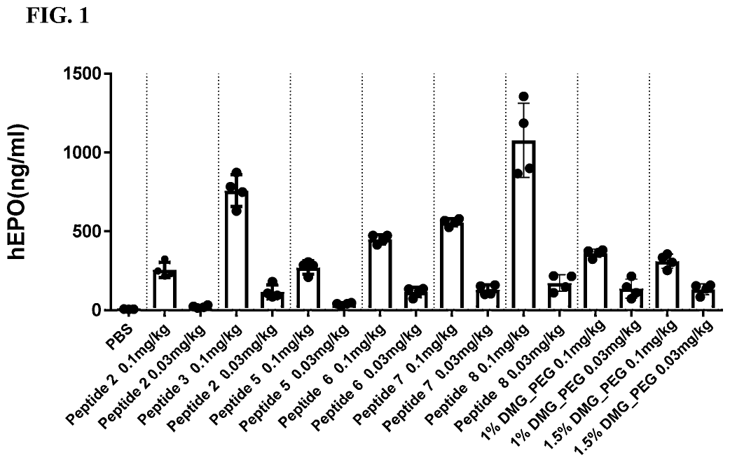

Each of the peptide-lipid conjugates was evaluated for its effectiveness in delivering hEPO mRNA for in vivo expression according to the protocol outlined in Example 4 at mRNA concentrations of 0.1 and 0.03 mg/kg. The PEG2000-DMG formulations were also tested at two different mole percent of the lipid portion of the composition of 1% and 1.5%. The results of this study are shown in FIG. 1. At the 0.1 mg/kg level, peptide 2 and peptide 5 formulations are comparable to the PEG2000-DMG formulations. The peptide 6 and peptide 7 show significantly higher EPO expression over the PEG2000-DMG formulations, while the peptide 3 and peptide 8 formulations show a far superior level of expression over the PEG2000-DMG formulations. These results show that the peptide-lipid conjugates of the present disclosure are at least suitable alternatives the use of PEG conjugates in lipid nanoparticles, and in some instances far superior in enhancing protein expression levels of mRNA delivered in vivo.

### Example 8: FVII Knockdown In Vivo

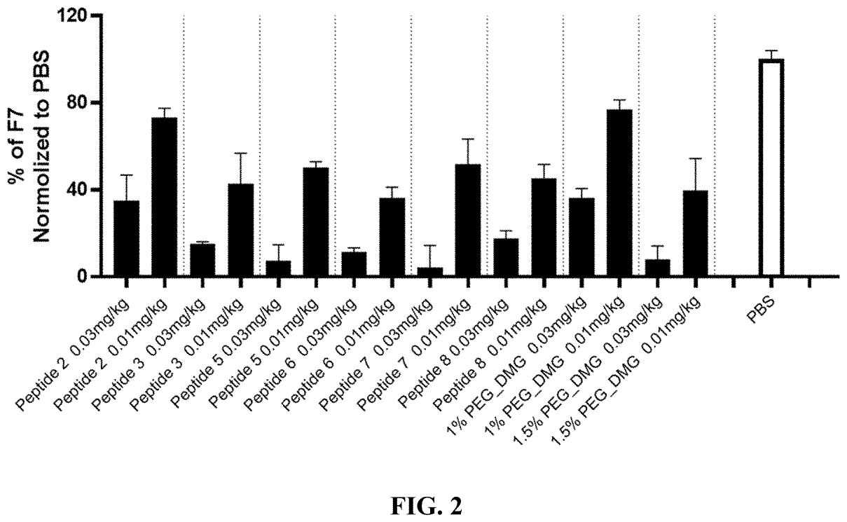

The peptide-lipid conjugates were further evaluated for effectiveness in knockdown of Factor VII (FVII Knockdown) by formulating lipid nanoparticles as described above encapsulating a siRNA targeted to knockdown FVII. These formulations were tested at FVII siRNA dose levels of 0.01 and 0.03 mg/kg. Comparative formulations that were otherwise identical as to lipid structure, but used either 1.0% or 1.5% PEG2000-DMG as well as a negative control of phosphate-buffered saline (PBS) were also tested. The results, normalized to PBS expression FVII expression levels, are provided in FIG. 2. It can be seen that Peptide 2 shows comparable expression levels to the 1% PEG-DMG formulations. Peptides 3, 5, 6, 7, and 8 all showed better knockdown activity than the 1% PEG-DMG formulations and were comparable to the 1.5% PEG-DMG formulations. Peptide 7 showed particularly improved knockdown at the 0.03 mg/kg dose level as compared to the 1.5% PEG-DMG formulations. Thus, the peptide-lipid conjugates of the present disclosure are at least suitable alternatives the use of PEG conjugates in lipid nanoparticles, and in some instances far superior in enhancing delivery and knockdown activity in vivo.

### Example 9: Further Peptide-Lipid Conjugates and Synthesis Thereof

Additional peptide-lipid conjugates were designed and described in this example as outlined in Table 3 and Schemes 2-8 below.

TABLE 3
Additional Peptide-Lipid Conjugates

| Peptide- | | |
|---|---|---|
| Lipid | | |
| Conjugate | | Molecular |
| Reference | Sequence (In an N-terminal to C-terminal Direction) | Weight |
| Peptide 9 | AcNH-STEPSTEPSTEPSTEP-X (Compound X is conjugated at C-termius and N- | 2283.59 |
| | terminus is capped with an acetyl group) | |
| Peptide 10 | AcNH-S(Me)T(Me)QPS(Me)T(Me)QPS(Me)T(Me)QPS(Me)T(Me)QP-X (Compound X | 2391.87 |
| | is conjugated at C-termius and N-terminus is capped with an acetyl group) | |
| Peptide 11 | X-STEP_{β}ASTEP_{β}ASTEP_{β}ASTEP-OH | 2483,79 |
| Peptide 12 | X-S(Me)T(Me)QP_{β}AS(Me)T(Me)QP_{β}AS(Me)T(Me)QP_{β}AS(Me)T(Me)QP-OH | 2592.07 |
| Peptide 13 | X-STEPSTEPSTEPSTEP-OH | 2144.40 |
| Peptide 14 | X-S(Me)T(Me)QPS(Me)T(Me)QPS(Me)T(Me)QPS(Me)T(Me)QP-OH | 2254.65 |
| Peptide 15 | X-STEPSTEPSTEPSTEP-OH | 2269,57 |
| Peptide 16 | X-S(Me)T(Me)QPS(Me)T(Me)QPS(Me)T(Me)QPS(Me)T(Me)QP-OH | 2377.85 |
| Peptide 17 | X-STEPSTEPSTEPSTEP-OH | 2314.70 |
| Peptide 18 | X-S(Me)T(Me)QPS(Me)T(Me)QPS(Me)T(Me)QPS(Me)T(Me)QP-OH | 2422.70 |
| Peptide 19 | X-STEPSTEPSTEPSTEP-OH | 2326.80 |
| Peptide 20 | X-S(Me)T(Me)QPS(Me)T(Me)QPS(Me)T(Me)QPS(Me)T(Me)QP-OH | 2434.80 |
| Peptide 21 | X-STEPSTEPSTEPSTEP-OH | 2382,90 |
| Peptide 22 | X-S(Me)T(Me)QPS(Me)T(Me)QPS(Me)T(Me)QPS(Me)T(Me)QP-OH | 2490.90 |

X = Is a compound (e.g., lipid with linker or a cholesterol with linker, etc.) conjugated to the peptide as provided in Schemes 2-8 below.
S = Serine
T = Threonine
E = Glutamic Acid
P = Proline, with P-OH representing proline, and P-NH₂ representing prolinamide, which was used to mask a negative charge at the C-terminus.
S(Me) = Methyl Serine
T(Me) = Methyl Threonine
Q = Glutamine
_{β}A = beta-Alanine

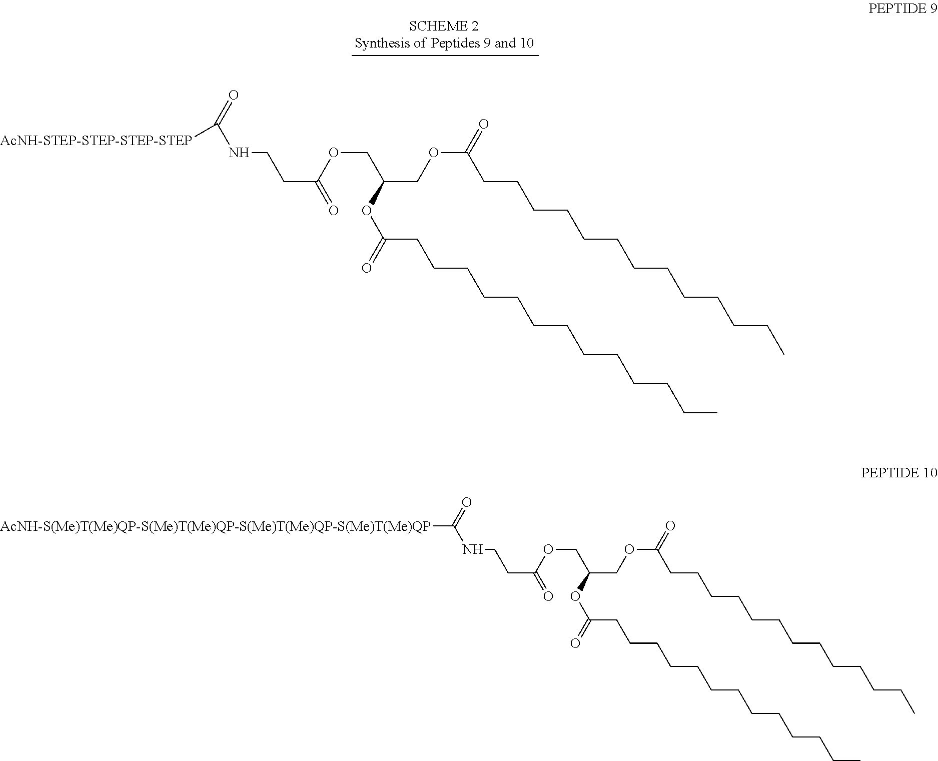

Synthesis of Intermediates for Peptides 9 and 10

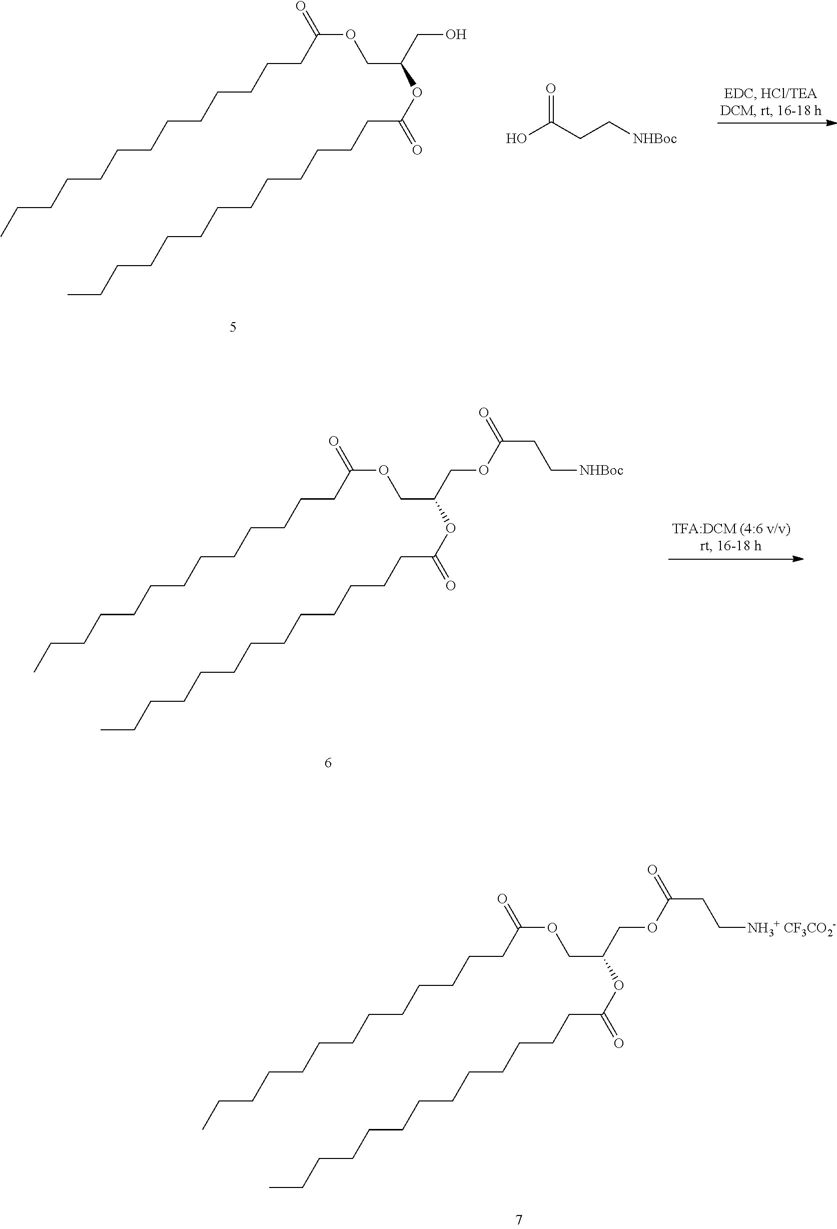

### Scheme 2, Step 1: (R)-3-((3-((tert-butoxycarbonyl)amino)propanoyl)oxy)propane-1,2-diyl ditetradecanoate (6)

[(2R)-3-hydroxy-2-tetradecanoyloxy-propyl] tetradecanoate (513 mg, 1 mmol), 3-(tert-butoxycarbonylamino)propanoic acid (227 mg, 1.2 mmol), EDC.HCl (238 mg, 1.3 mmol) and triethylamine (0.21 mL, 1.7 mmol) were mixed in 5 mL dichloromethane and stirred overnight. Diluted with another 5 mL dichloromethane and washed with 1N HCl (1×10 mL) followed by water (1×10 mL), dried (Na₂SO₄), filtered and concentrated under reduced pressure. The crude product was purified on silica gel column (TELEDYNE ISCO gold, 12 g) using dichloromethane/ethyl acetate gradient (0-60% over 15 minutes). Product eluted at 15-20% ethyl acetate concentration gradient was collected, analyzed and concentrated under reduced pressure to afford 540 mg (79%) pure product. m/z 684.0 (Calculated) M−H+Na 706.4 (Observed).

### Scheme 2, Step 2: (R)-3-((3-aminopropanoyl)oxy)propane-1,2-diyl ditetradecanoate (7)

Boc protected compound [(2R)-3-[3-(tert-butoxycarbonylamino)propanoyloxy]-2-tetradecanoyloxy-propyl] tetradecanoate (500 mg, 0.73 mmol) was taken in 6 mL dichloromethane and 4 mL TFA was added. The mixture was stirred at rt overnight. Solvent was evaporated and the residue was purified on silica gel column using dichloromethane/Methanol gradient (0-60% over 15 minutes). Product eluted at 20% Methanol was collected, concentrated under vacuum and dried to get pure product (360 mg, 84%) that was used in the coupling to peptide. m/z 583.9 (Calculated) M 584.3 (Observed).

Compound 7 can be coupled to the C-terminus of a pre-synthesized STEP peptide sequence that is derivatized at the N-terminus with an acetyl group and the glutamic acid side chain carboxylic acids are protected with benzyl ester as is known in the peptide synthesis protocol using Boc-Glu(OBz)-OH, using standard coupling agents such as diisopropylcarbodimide (DIC) and 1-hydroxybenzotriazole (HOBt) reagents. If Fmoc chemistry is used in the peptide synthesis, these amino acid side chains can be typically protected as tert-butyl ester Fmoc-Glu(OtBu)-OH. In the end, such side chain protection groups can be removed under either hydrogenation conditions or using formic acid or trifluoroacetic acid to get the crude peptides 9 and 10 which may be purified on C4 column as explained previously.

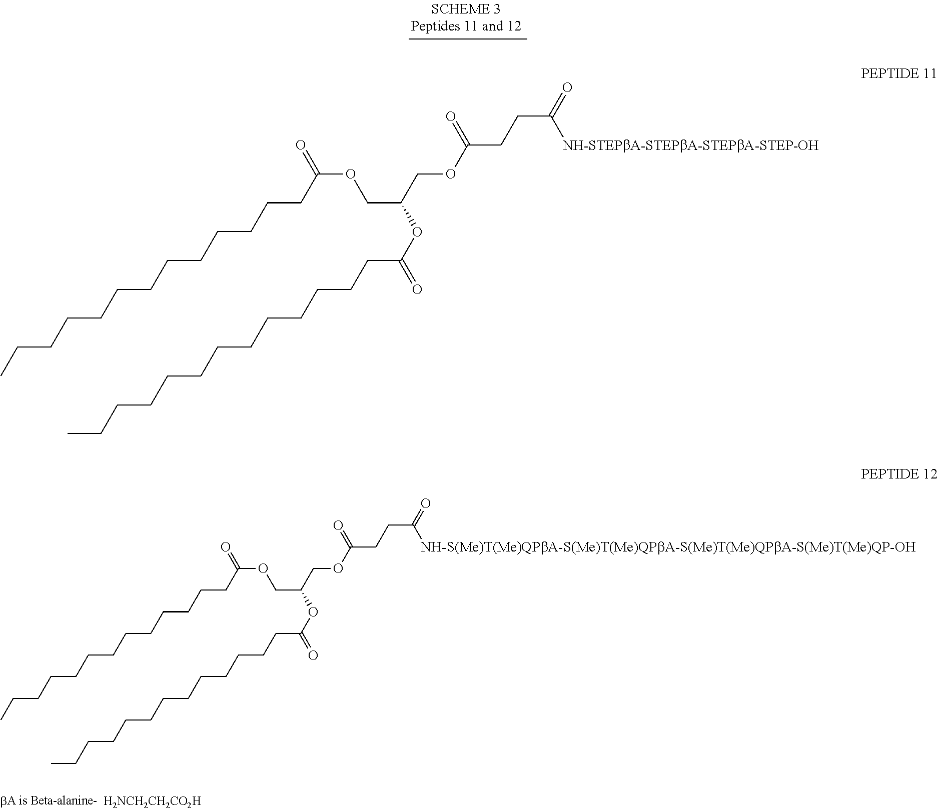

Synthesis of Intermediates for Peptides 11 and 12

The intermediate for Peptides 11 and 12 are the same as for Peptides 1-8 provided in Example 1, namely intermediate 3 as shown below.

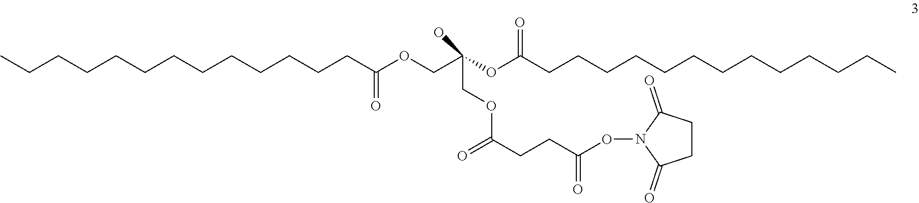 [SMILES: CCCCCCCCCCCCCC(=O)OC[C@@](O)(COC(=O)CCC(=O)ON1C(=O)CCC1=O)OC(=O)CCCCCCCCCCCCC]

Peptides containing an additional O3-alanine at the C-terminal end of each STEP or S(Me)T(Me)QP segment as shown for Peptides 11 and 12 can be synthesized and the N-terminal end of such peptides can be coupled to 3 following protocols developed for Peptides 1-8 of Example i to get crude Peptides 11 and 12, which may be purified on a C4 hydrophobic interaction column as explained previously.

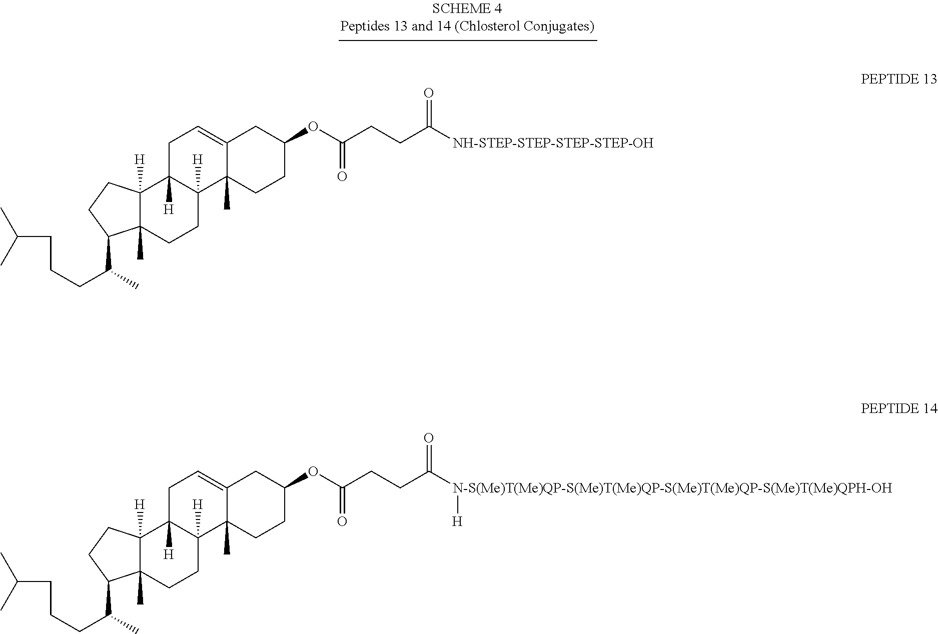

Commercially available cholesterol NHS hemisuccinate (CAS #88848-79-7) can be used as such in the coupling of pure peptides to get Peptide 13 and Peptide 14 following coupling protocol established for Peptides 1-8 of Example 1.

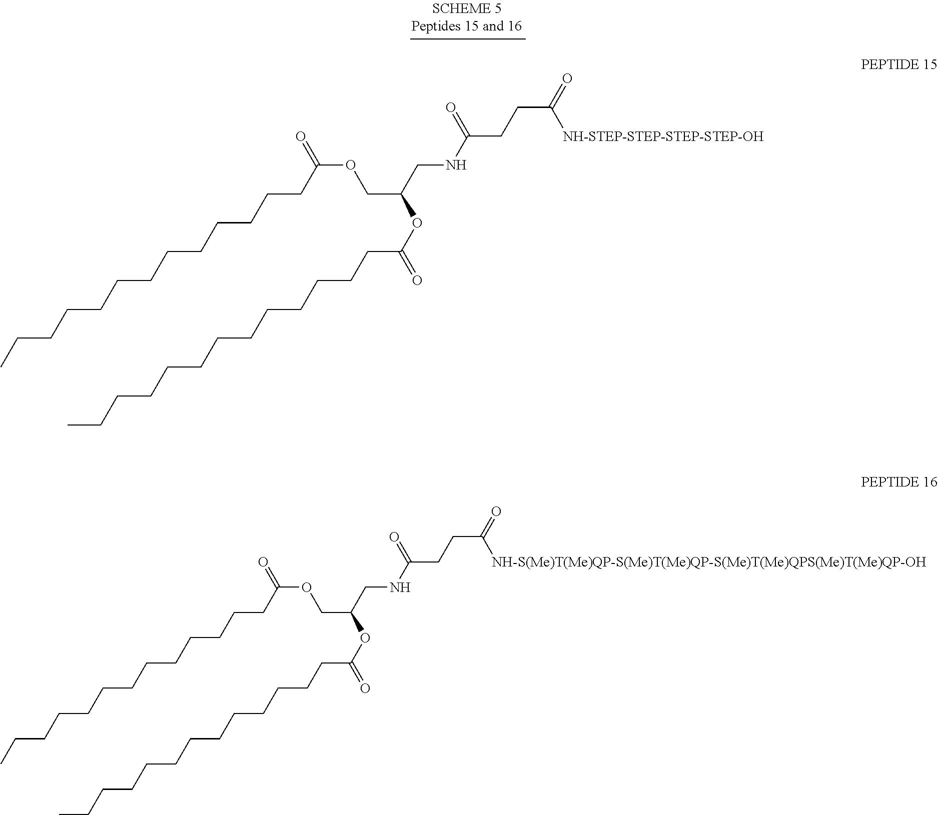

Synthesis of Intermediates for Peptides 15 and 16

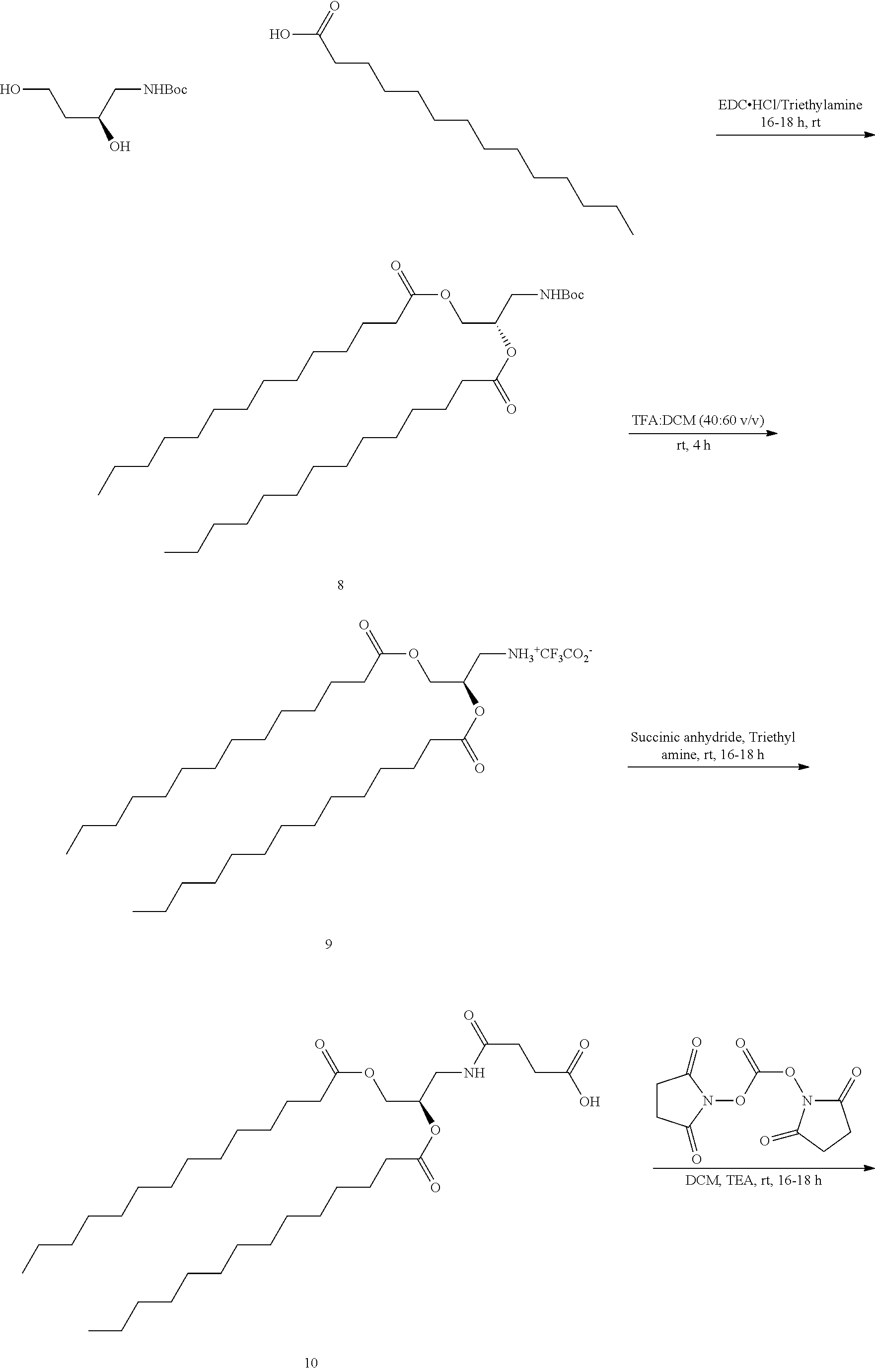

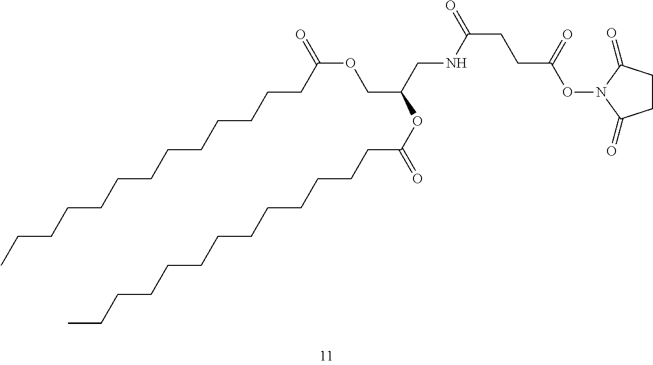 [SMILES: CCCCCCCCCCCCCC(=O)OC[C@@H](CNC(=O)CCC(=O)ON1C(=O)CCC1=O)OC(=O)CCCCCCCCCCCCC]

### Scheme 5, Step 1: (R)-3-((tert-butoxycarbonyl)amino)propane-1,2-diyl ditetradecanoate (8)

To solution of tert-butyl N-[(2R)-2,3-dihydroxypropyl]carbamate (0.5 g, 2.6 mmol) in dichloromethane (12 mL) was added tetradecanoic acid (1.8 g, 7.8 mmol), EDC (1.1 g, 5.5 mmol) followed by triethylamine (0.82 mL, 5.9 mmol). The mixture was stirred at rt overnight. Solution was diluted with dichloromethane (15 mL) and washed with 1N HCl (2×15 mL), water (2×15 mL), dried (Na₂SO₄), filtered and evaporated under reduced pressure. The residue was purified on silica gel column using hexane/ethyl acetate. Product eluted at 30% ETHYL ACETATE. m/z 611.9 (Calculated) M−H+Na 634.4 (Observed).

### Scheme 5, Step 2: (R)-3-aminopropane-1,2-diyl ditetradecanoate (9)

A solution of IK473 (1.4 g) in a 40% TFA in dichloromethane (V/V) was stirred at room temperature for 4 hours. TLC analysis showed reaction completion. Solvent was evaporated under reduced pressure and the material obtained was used as such in the next reaction without further purification. m/z 511.8 (Calculated) M 512.4 (Observed).

### Scheme 5, Step 3: (R)-4-((2,3-bis(tetradecanoyloxy)propyl)amino)-4-oxobutanoic acid (10)

To a solution of 9 in dichloromethane was added [(2R)-3-amino-2-tetradecanoyloxy-propyl]tetradecanoate followed by tetrahydrofuran-2,5-dione and diisopropyethyl amine and the mixture was stirred at rt overnight. TLC (10% methanol in dichloromethane) showed two faster moving spots upon iodine/silica gel treatment. Evaporated and loaded onto TELEDYNE ISCO gold silica gel column and eluted with 0-60% methanol gradient in dichloromethane over 15 minutes. Fractions eluted were isolated, analyzed, pooled, and evaporated under reduced pressure. m/z 611.9 (Calculated) M−H+Na 634.4 (Observed).

Scheme 5, Step 4: (R)-3-(4-((2,5-dioxopyrrolidin-1-yl)oxy)-4-oxobutanamido)propane-1,2-diyl ditetradecanoate (11). To a solution of 4-[[(2R)-2,3-di(tetradecanoyloxy)propyl]amino]-4-oxo-butanoic acid (404 mg, 0.66 mmol) in 4 ml dichloromethane was added bis(2,5-dioxopyrrolidin-1-yl) carbonate (338 mg, 1.3 mmol) followed by triethylamine (0.23 mL, 1.7 mmol). The mixture was stirred overnight and diluted with dichloromethane (4 mL), washed with ice-cold water (10 mL), dichloromethane solution was isolated and dried with Na₂SO₄, filtered, and evaporated under reduced pressure. The crude product was loaded onto 12 g Teledyne ISCO gold column with 3 mL dichloromethane and eluted with gradient of 0-60% EtOAc in hexane over 15 minutes. Product containing fractions were pooled, concentrated under reduced pressure, and dried to get 360 mg (77%) product as a white solid. m/z 709 (Calculated) M−H 708.1 (Observed).

Intermediate 11 can be used in the coupling of peptides at the N-terminal following the protocol developed for peptides 1-8 to get Peptide 15 and Peptide 16.

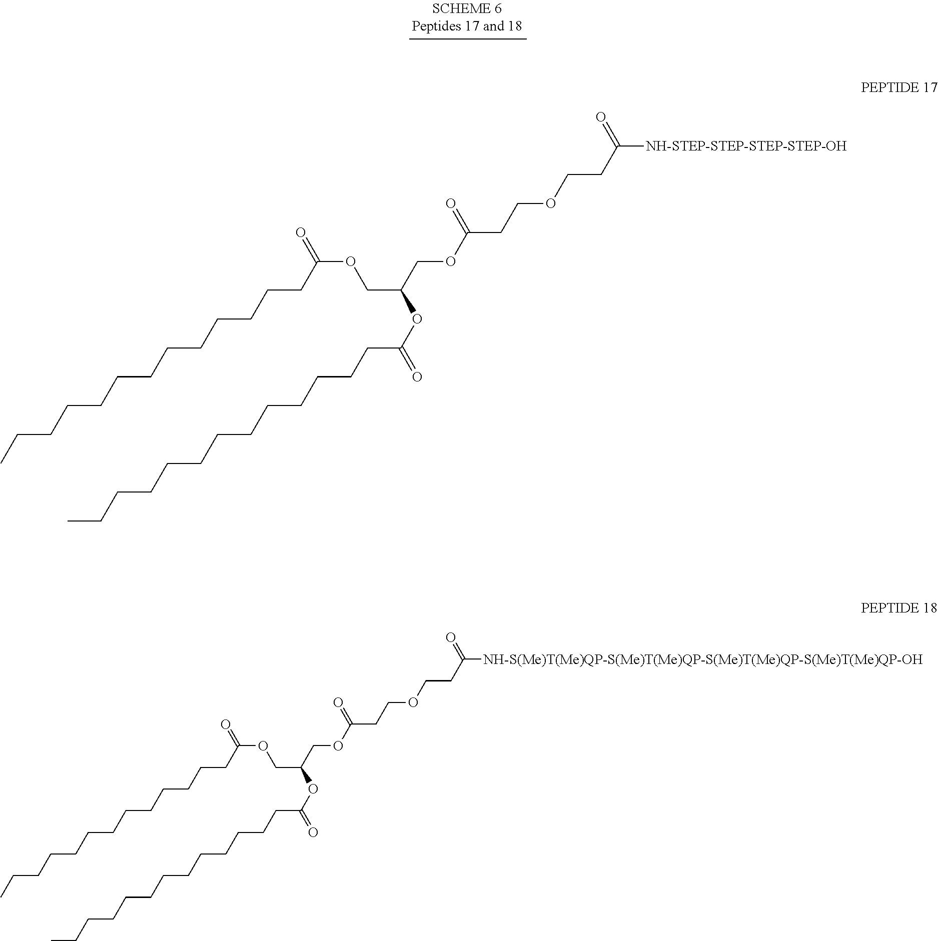

Synthesis of Intermediates for Peptides 17 and 18

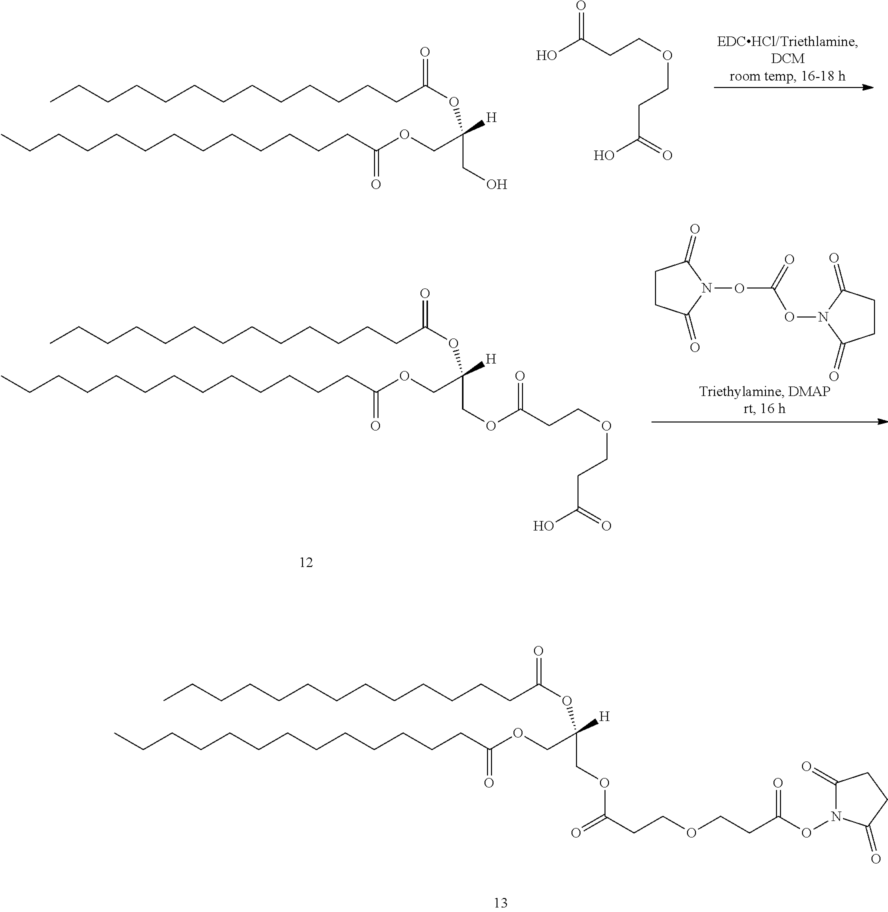 [SMILES: CCCCCCCCCCCCCC(=O)OC[C@@H](CO)OC(=O)CCCCCCCCCCCCC ; O=C(O)CCOCCC(=O)O ; CCCCCCCCCCCCCC(=O)OC[C@@H](COC(=O)CCOCCC(=O)O)OC(=O)CCCCCCCCCCCCC ; O=C(ON1C(=O)CCC1=O)ON1C(=O)CCC1=O ; CCCCCCCCCCCCCC(=O)OC[C@@H](COC(=O)CCOCCC(=O)ON1C(=O)CCC1=O)OC(=O)CCCCCCCCCCCCC]

### Scheme 6, Step 1: (S)-3-(3-(2,3-bis(tetradecanoyloxy)propoxy)-3-oxopropoxy)propanoic acid (12)

A mixture of 2 g (3.9 mmol) of [(2R)-3-hydroxy-2-tetradecanoyloxy-propyl]tetradecanoate, 561 mg (2.9 mmol) of EDC.HCl, 0.82 mL (1.5 mmol) triethylamine and 474 mg (0.75 mmol) of 3-(2-carboxyethoxy)propanoic acid in 10 mL dichloromethane was stirred at room temperature overnight. The mixture was diluted with 5 mL dichloromethane and washed with 10 mL water, followed by brine (10 mL), dried over anhydrous sodium sulphate, filtered, and concentrated under reduced pressure. The crude product was purified on silica gel column (Teledyne ISCO gold 12 g) with dichloromethane/ethyl acetate gradient (0-100% ethyl acetate) and the product eluted at 40% ethyl acetate was collected and concentrated under reduced pressure to get 1.2 g (47%) product. m/z 656.4 (Calculated) M−H 655.2 (Observed).

### Scheme 6, Step 2: (S)-3-((3-(3-((2,5-dioxopyrrolidin-1-yl)oxy)-3-oxopropoxy)propanoyl)oxy)propane-1,2-diyl ditetradecanoate (13)

A mixture of 3-[3-[(2S)-2,3-di(tetradecanoyloxy)propoxy]-3-oxo-propoxy]propanoic acid (525 mg, 0.80 mmol), bis(2,5-dioxopyrrolidin-1-yl) carbonate (409 mg, 1.6 mmol) and triethylamine (0.28 mL, 2 mmol) in 4 mL dichloromethane was stirred overnight. Reaction mixture was diluted with dichloromethane (4 mL) and washed with ice-cold water (10 mL), dichloromethane solution was isolated and dried with Na₂SO₄, filtered and evaporated. The crude product was loaded onto 12 g Teledyne ISCO gold column with 3 mL dichloromethane and eluted with gradient of 0-60% ethyl acetate in Hexane over 15 minutes. A product eluted at 20-25% ethyl acetate was collected, concentrated under reduced pressure, and dried under vacuum to get 350 mg (58%) pure product. m/z 754.0 (Calculated) M−H+Na 776.2 (Observed).

Intermediate 13 was used in the preparation of Peptide 17 and Peptide 18 following the coupling and purification protocol used for Peptides 1-8 as described in Example 1.

Peptide 17: HPLC purity 92%. Mass: 2314.7 (calcd.), 2314.8 (Observed).

Peptide 18: HPLC purity 100%. Mass: 2422.7 (calcd.), 2422.8 (Observed).

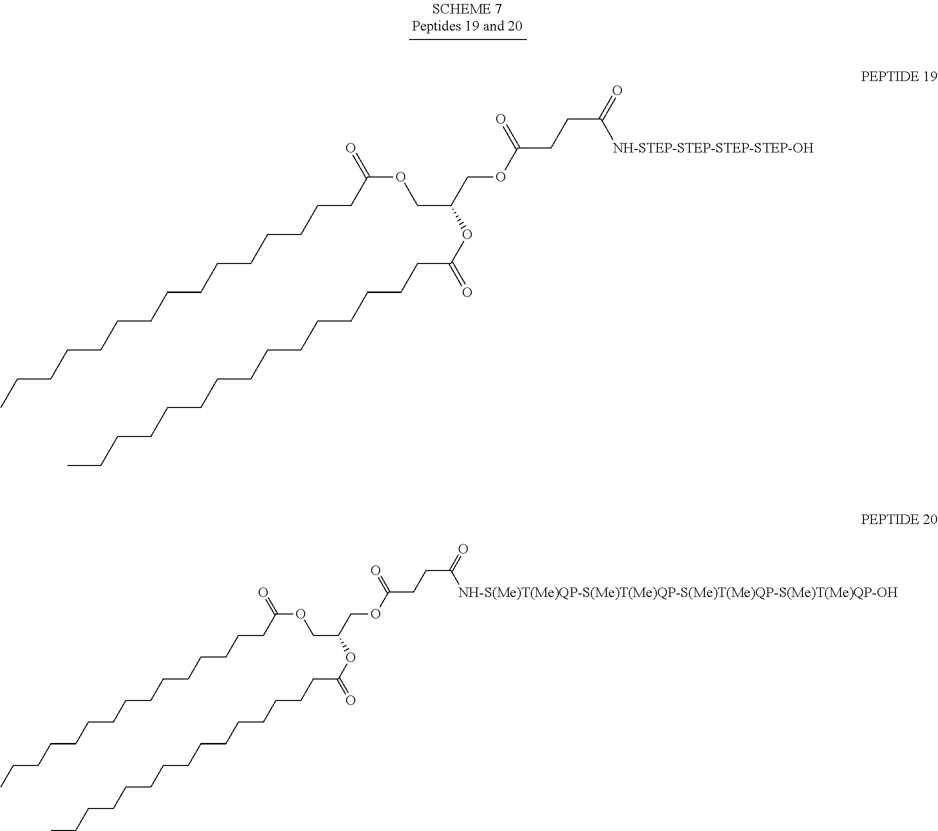

Synthesis of Intermediates for Peptides 19 and 20

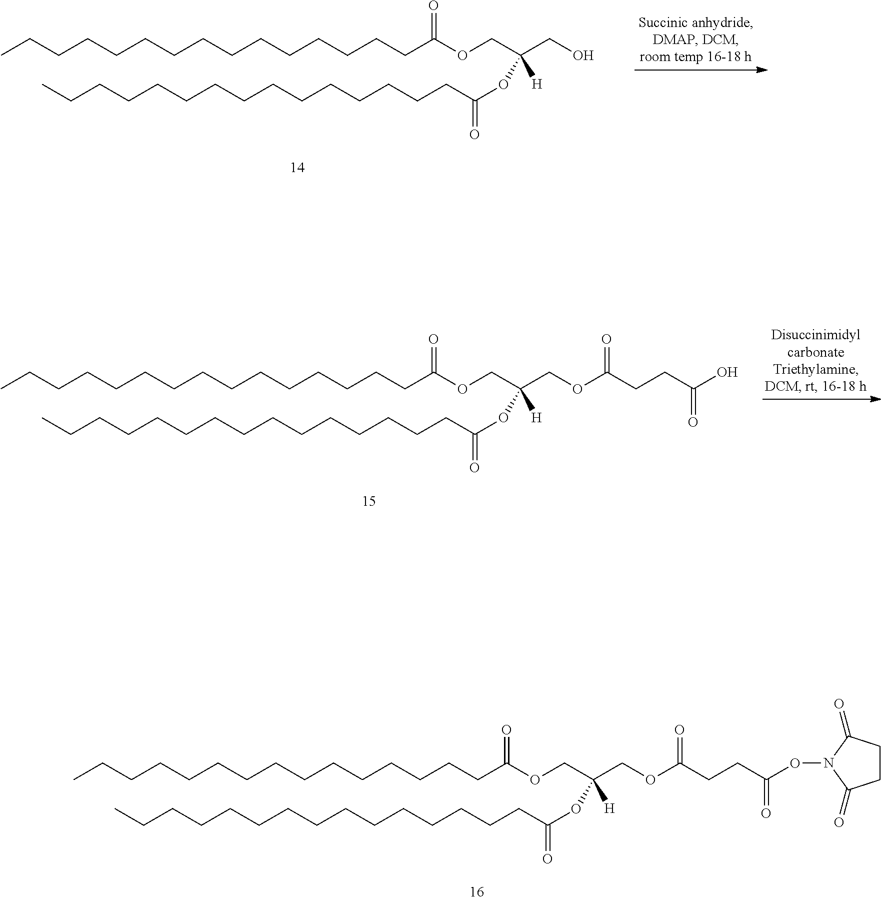 [SMILES: CCCCCCCCCCCCCCCC(=O)OC[C@H](CO)OC(=O)CCCCCCCCCCCCCCC ; CCCCCCCCCCCCCCCC(=O)OC[C@H](COC(=O)CCC(=O)O)OC(=O)CCCCCCCCCCCCCCC ; CCCCCCCCCCCCCCCC(=O)OC[C@H](COC(=O)CCC(=O)ON1C(=O)CCC1=O)OC(=O)CCCCCCCCCCCCCCC]

### Scheme 7, Step 1: (R)-4-(2,3-bis(palmitoyloxy)propoxy)-4-oxobutanoic acid (15)

To a suspension of 1,2-dipalmytoyl-sn-glycerol (2 g, 3.2 mmol) in 40 mL anhydrous dichloromethane in 200 mL RB flask under argon kept in ice bath was added 563 mg (5.6 mmol) of succinic anhydride followed by 902 mg (7.4 mmol) of DMAP. The mixture was allowed to come to room temperature and stirred at room temperature overnight. TLC analysis (10% methanol/dichloromethane) showed a slower moving spot along with DMAP at the bottom. The mixture was washed with 1N HCl (3×30 mL), water and brine (100 ml each), dried (Na₂SO₄), filtered and evaporated. Column purification (Teledyne ISCO 40 g) with methanol/dichloromethane gradient eluted product at 12-15% Methanol. Concentration of fractions afforded 2 g (85%) of product as a white solid. m/z 668.5 (Calculated) M−H 667.5 (Observed).

### Scheme 7, Step 2: (R)-2,3-bis(palmitoyloxy)propyl (2,5-dioxopyrrolidin-1-yl) succinate (16)

To a mixture of 4-[(2R)-2,3-di(hexadecanoyloxy)propoxy]-4-oxo-butanoic acid (2 g, 3 mmol) triethylamine (0.83 mL, 6 mmol) and DMAP (50 mg, cat.) in 40 mL anhydrous dichloromethane was added bis(2,5-dioxopyrrolidin-1-yl) carbonate (1.15 g, 4.5 mmol) and the mixture was stirred at room temperature overnight. Two equivalents of acetic acid were added to quench the reaction. The mixture was diluted with dichloromethane and washed with ice-cold water (2×80 mL) followed by brine (80 mL), dried (Na₂SO₄) and evaporated under reduced pressure. The residue was purified on silica gel column (Teledyne ISCO 40) using dichloromethane:ethyl acetate gradient (0-40% over 30 minutes). The product eluted at 10-12% ethyl acetate. Solvent was removed under rotary evaporator and the white solid obtained was dried under vacuum to get 1.6 g of product. m/z 765.5 (Calculated) M+H 788.5 (Observed).

Intermediate 16 was used in the preparation of Peptide 19 and Peptide 20 following the coupling and purification protocol used for Peptides 1-8 as described in Example 1.

Peptide 19: Mass: 2326.8 (calcd.), 2326.0 (Observed).

Peptide 20: Mass: 2434.8 (calcd.), 2434.0 (Observed).

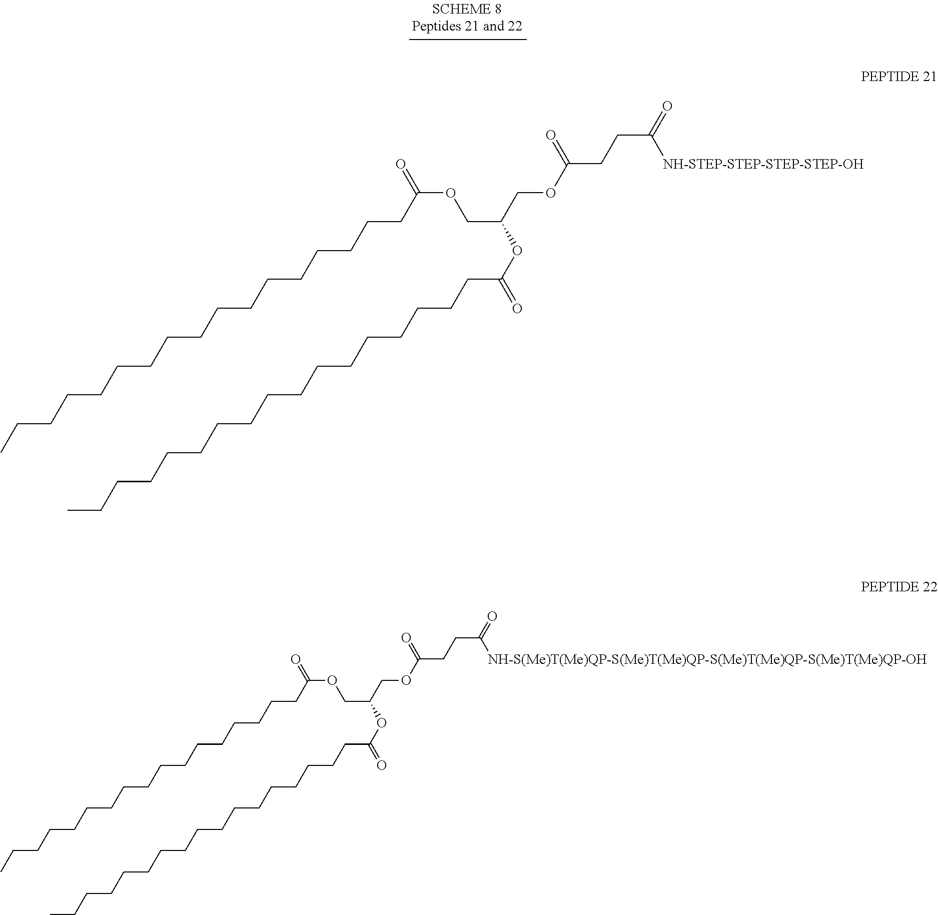

Synthesis of Intermediates for Peptides 21 and 22

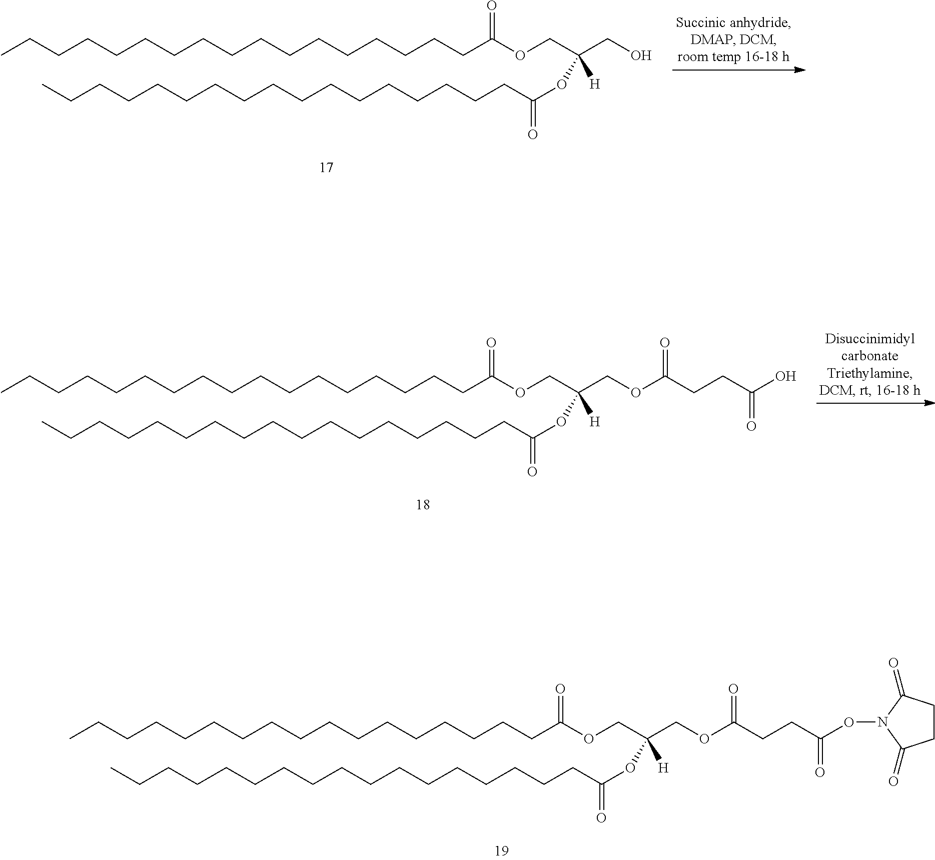 [SMILES: CCCCCCCCCCCCCCCCCC(=O)OC[C@H](CO)OC(=O)CCCCCCCCCCCCCCCCC ; CCCCCCCCCCCCCCCCCC(=O)OC[C@H](COC(=O)CCC(=O)O)OC(=O)CCCCCCCCCCCCCCCCC ; CCCCCCCCCCCCCCCCCC(=O)OC[C@H](COC(=O)CCC(=O)ON1C(=O)CCC1=O)OC(=O)CCCCCCCCCCCCCCCCC]

### Scheme 8, Step 1: (R)-4-(2,3-bis(stearoyloxy)propoxy)-4-oxobutanoic acid (18)

To a suspension of (S)-3-hydroxypropane-1,2-diyl distearate (2 g, 3.2 mmol) in 40 mL anhydrous dichloromethane in 200 mL RB flask under argon kept in ice bath was added 512 mg (5.6 mmol) of succinic anhydride followed by 821 mg (7.4 mmol) of DMAP. The mixture was allowed to come to room temperature and stirred at rt overnight. TLC analysis (10% Methanol/dichloromethane) showed a slower moving spot along with DMAP at the bottom. The mixture was washed with 1N HCl (3×30 mL), water and brine (100 ml Ethyl acetatech), dried (Na₂SO₄), filtered and evaporated. Column purification (Teledyne ISCO 80 g) with methanol/dichloromethane gradient eluted product at 12-15% Methanol. Concentration of fractions afforded 2 g (86%) of product as a white solid. m/z 724.5 (Calculated) M−H 723.5 (Observed).

### Step 2: (R)-2,3-bis(stearoyloxy)propyl (2,5-dioxopyrrolidin-1-yl) succinate (19)

To a mixture of (R)-4-(2,3-bis(stearoyloxy)propoxy)-4-oxobutanoic acid (2 g, 2.8 mmol) triethylamine (0.77 mL, 5.5 mmol) and DMAP (50 mg, cat.) in 30 mL anhydrous dichloromethane was added bis(2,5-dioxopyrrolidin-1-yl) carbonate (1.1 g, 4.1 mmol) and the mixture was stirred at room temperature overnight. Two equivalents of acetic acid were added to quench the reaction. The mixture was diluted with dichloromethane and washed with ice-cold water (2×80 mL) followed by brine (80 mL), dried (Na₂SO₄) and evaporated under reduced pressure. The residue was purified on silica gel column (Teledyne ISCO 40) using dichloromethane:ethyl acetate gradient (0-40% over 30 minutes). The product eluted at 10-12% ethyl acetate. Solvent was removed under rotary evaporator and the white solid obtained was dried to get 1.5 g (66%) of product. m/z 822.5 (Calculated) M+Na 845.5 (Observed).

Intermediate 19 was used in the preparation of Peptide 21 and Peptide 22 following the coupling and purification protocol used for Peptides 1-8 as described in Example 1.

Peptide 21: Mass: 2382.9 (calcd.), 2382.0 (Observed).

Peptide 22: Mass: 2490.9 (calcd.), 2491.0 (Observed).

### ABBREVIATIONS USED

DCM: Dichloromethane

DMAP: N,N-Dimethylpyridine

DMG: Dimyristoyl glycerol

DPG: Dipalmitoyl glycerol

DSG: Distearoyl glycerol

EA: Ethyl acetate

EDC.HCl: 1-(3-Dimethylaminopropyl)-3-ethylcarbodiimide hydrochloride

HCl: Hydrochloric acid

TEA: Triethylamine

TFA: Trifluoroacetic acid

TLC: Thin layer chromatography

### Example 10: Further Peptide-Lipid Conjugates and Synthesis Thereof

Selected peptide-lipid conjugates from Example 9 were formulated into lipid nanoparticles and characterized following the methods and protocols described in Example 2. The lipid nanoparticles showed good particle size, dispersion, and encapsulation as shown in the data of Table 4 below. These lipid nanoparticle formulations included an ionizable cationic lipid (“Cat”), helper lipid (distearoylphosphatidylcholine, “DSPC”), cholesterol (“Chol”), and the indicated lipid-peptide conjugate. The ionizable cationic lipid used in these formulations was selected to provide a common lipid that could serve as a basis for comparison, however a person of skill in the art would recognize that the lipid-peptide conjugates of the disclosure can be combined with any cationic lipid suitable for use in a lipid nanoparticle formulation for the delivery of an active agent such as a nucleic acid. The ionizable cationic lipid used in these formulations has the following structure:

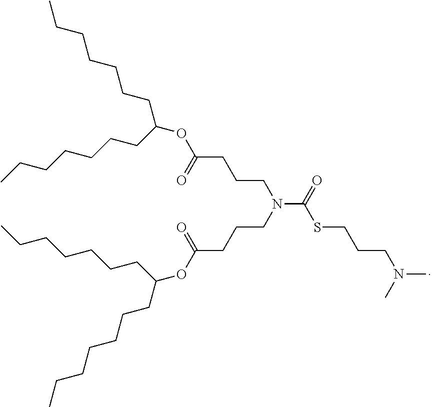 [SMILES: CCCCCCCC(CCCCCCC)OC(=O)CCCN(CCCC(=O)OC(CCCCCCC)CCCCCCC)C(=O)SCCCN(C)C]

The lipid nanoparticle formulations were prepared and characterized as described in Example 2, the details of each formulation together with the resultant characteristics are provided in Table 4 below. In this table, “N/P” refers to the ratio of cationic amino groups from the ionizable cationic lipid to the anionic phosphate backbone groups of the encapsulated nucleic acid. The results indicate that the peptide-lipid conjugates of the disclosure integrate well into lipid nanoparticle formulations with good particle size, polydispersity, and percent encapsulation of the nucleic acid.

The formulations are further tested for in vivo measurement of hEPO expression following the protocol outlined in Example 7.

TABLE 4
Formulation Data for Selected Peptides

| | | Diameter | | Percent |
|---|---|---|---|---|
| Lipid Composition | Nucleic Acid | (nm) | Polydispersity | Encapsulation |
| Cat:DSPC:Chol:Peptide 17 | hEPO mRNA | 60.19 | 0.194 | 94.6 |
| molar ratio | | | | |
| 40:15:44:1; N/P = 9 | | | | |
| Cat:DSPC:Chol:Peptide 18 | hEPO mRNA | 54.26 | 0.25 | 93.5 |
| molar ratio | | | | |
| 40:15:44:1; N/P = 9 | | | | |
| Cat:DSPC:Chol:Peptide 19 | hEPO mRNA | 67.22 | 0.179 | 94.8 |
| molar ratio | | | | |
| 40:15:44:1; N/P =9 | | | | |
| Cat:DSPC:Chol:Peptide 20 | hEPO mRNA | 55.37 | 0.211 | 84.4 |
| molar ratio | | | | |
| 40:15:44:1; N/P = 9 | | | | |
| Cat:DSPC:Chol:Peptide 21 | hEPO mRNA | 88.29 | 0.174 | 91 |
| molar ratio | | | | |
| 40:15:44:1; N/P = 9 | | | | |
| Cat:DSPC:Chol:Peptide 22 | hEPO mRNA | 55.31 | 0.22 | 89.4 |
| molar ratio | | | | |
| 40:15:44:1; N/P = 9 | | | | |

### FURTHER CONSIDERATIONS

The examples above demonstrate that peptide-lipid conjugates are at least an adequate, and in some examples superior, substitute for PEG-lipids in lipid formulated nucleic acid delivery systems. These peptide-lipid conjugates showed comparative or superior levels of delivering mRNA for in vivo expression and siRNA for knockdown of a target. When the peptide-lipid conjugates further comprise a threshold amount of hydrophilic amino acids as described hereinabove, they provide the further effect of being able to formulate lipid compositions in aqueous media of suitable morphology, size, and dispersion. These hydrophilic side chains allow the peptide portion of the lipid conjugates at the exterior surface of the composition to maximize the association of the peptide with an aqueous medium thereby providing more ideal conformational structure (e.g., “water cage”). In addition, when the peptide-lipid conjugates have a threshold amount of proline, further conformational benefits are provided by allowing the proper amount of structural rigidity to the peptide portion of the conjugate. Likewise, minimizing glycine allows for the peptide portion of the peptide-lipid conjugate to have less variability in the overall conformational structure and provide the effect that only the more suitable conformations for maintaining a uniform morphology, size, and dispersion of the resulting compositions are produced.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

Furthermore, to the extent that the term “include,” “have,” or the like is used in the description or the claims, such term is intended to be inclusive in a manner similar to the term “comprise” as “comprise” is interpreted when employed as a transitional word in a claim.

In one or more aspects, the terms “about,” “substantially,” and “approximately” may provide an industry-accepted tolerance for their corresponding terms and/or relativity between items, such as from less than one percent to 5 percent.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a composition or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

1. A lipid composition containing a nucleic acid, wherein the lipid composition comprises a peptide-lipid conjugate.

2. The lipid composition of claim 1, wherein the peptide of the peptide-lipid conjugate consists of about 4 to about 52 amino acids.

3.-15. (canceled)

16. The lipid composition of claim 1, wherein at least about 14% of the amino acids in the peptide of the peptide-lipid conjugate are proline.

17.-19. (canceled)

20. The lipid composition of claim 1, wherein about 28% to about 80% of the amino acids in the peptide of the peptide-lipid conjugate have a hydrophilic side chain.

21.-25. (canceled)

26. The lipid composition of claim 20, wherein the amino acids having a hydrophilic side chain are selected from glutamine, glutamic acid, asparagine, aspartic acid, serine, O—C₁₋₆ alkyl serine, threonine, and O—C₁₋₆ alkyl threonine.

27. The lipid composition of claim 1, wherein less than about 43% of the amino acids in the peptide of the peptide-lipid conjugate are glycine.

28.-31. (canceled)

32. The lipid composition of claim 1, wherein the peptide does not have glycine.

33. The lipid composition of claim 1, wherein the lipid composition is selected from a lipoplex, a liposome, a lipid nanoparticle, a polymer-based carrier, an exosome, a lamellar body, a micelle, and an emulsion.

34.-36. (canceled)

37. The lipid composition of claim 1, wherein the lipid composition comprises one or more cationic lipids, one or more helper lipids, and a sterol.

38. The lipid composition of claim 1, wherein the peptide-lipid conjugate comprises about 0.1 mol % to about 10 mol % of all lipids in the lipid composition.

39. (canceled)

40. (canceled)

41. The lipid composition of claim 1, wherein the lipid composition encapsulates the nucleic acid.

42. (canceled)

43. The lipid composition of claim 1, wherein the lipid of the peptide lipid-conjugate is conjugated to the peptide via a linker.

44. The lipid composition of claim 43, wherein the linker has a structure comprising a group selected from amido (—C(O)NH—), amino (—NR^{N}—) wherein R^{N} is selected from H, C₁₋₆ alkyl, carbonyl (—C(O)—), carbamate (—NHC(O)O—), urea (—NHC(O)NH—), disulfide
(—S—S—), ether (—O—), succinyl (—(O)CCH₂CH₂C(O)—), succinamidyl (—NHC(O)CH₂CH₂C(O)NH—), ether, carbonate (—OC(O)O—), succinoyl, phosphate esters (—O—(O)POH—O—), and sulfonate esters.

45. The lipid composition of claim 1, wherein the lipid of the peptide-lipid conjugate is selected from a didecyloxypropyl (C₁₀), a dilauryloxypropyl (C₁₂), a dimyristyloxypropyl (C₁₋₆), a dipalmityloxypropyl (C₁₆), or a distearyloxypropyl (C₁₈), a 1,2-dimyristyloxypropyl-3-amine (DOMG), a 1,2-dimyristyloxypropylamine (DMG), a 1,2-Dilauroyl-sn-glycero-3-phosphorylethanolamine (DLPE), a dimyristoyl-phosphatidylethanolamine (DMPE), a dipalmitoyl-phosphatidylethanolamine (DPPE), a dipalmitoylphosphatidylcholine (DPPC), a dioleoyl-phosphatidylethanolamine (DOPE), a distearoyl-phosphatidylethanolamine (DSPE), and cholesterol or a cholesterol derivative.

46. The lipid composition of claim 1, wherein the peptide is conjugated to the lipid of the peptide-lipid conjugate at its C-terminus and the amino group of the N-terminus of the peptide is substituted with one or two C₁₋₆ alkyl groups or amido groups.

47. The lipid composition of claim 1, wherein the peptide is conjugated to the lipid of the peptide-lipid conjugate at its N-terminus, and the amino acid at the C-terminus of the peptide is alkylated to form a C₁₋₆ alkyl ester or is amidated.

48. The lipid composition of claim 1, further comprising one or more cationic lipids selected from 5-carboxyspermylglycinedioctadecylamide (DOGS), 2,3-dioleyloxy-N-[2(spermine-carboxamido)ethyl]-N,N-dimethyl-1-propanaminium (DOSPA), 1,2-Dioleoyl-3-Dimethylammonium-Propane (DODAP), 1,2-Dioleoyl-3-Trimethylammonium-Propane (DOTAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-oc-tadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), and 2,2-dilinoleyl-4-dimethylaminoethyl-[1,3]-dioxolane or (DLin-K-XTC2-DMA).

49. (canceled)

50. The lipid composition of claim 1, further comprising one or more helper lipids.

51. A lipid composition comprising a peptide-lipid conjugate and a nucleic acid, wherein:
a. the peptide of the peptide-lipid conjugate consists of about 4 to about 52 amino acids;
b. at least about 14% of the amino acids in the peptide of the peptide-lipid conjugate are proline;
c. about 28% to about 80% of the amino acids in the peptide of the peptide-lipid conjugate have a hydrophilic side chain;
d. less than about 43% of the amino acids in the peptide of the peptide-lipid conjugate are glycine;
e. the lipid composition comprises one or more cationic lipids, one or more helper lipids, and a sterol; and
f. the peptide-lipid conjugate comprises about 0.1 mol % to about 10 mol % of all lipids in the lipid composition.

52. The lipid composition of claim 1, wherein the nucleic acid is selected from a siRNA, an antisense oligonucleotide, a UNA oligomer, an mRNA, a micro RNA, and a DNA.

53. (canceled)

54. The lipid composition of claim 5, wherein the mRNA is a self-replicating mRNA.

55.-61. (canceled)

62. A method of treating a disease in a subject in need thereof comprising administering a lipid composition of claim 1.

63. A method of expressing a protein or polypeptide of interest in a cell comprising contacting the cell with a lipid composition of claim 1, wherein the nucleic acid is a mRNA or a self-replicating mRNA.

64. A vaccine comprising a lipid composition of claim 1, wherein the nucleic acid is a mRNA or a self-replicating mRNA.

65. A method of inducing an immune response in a subject comprising administering a vaccine of claim 64 to the subject.

66.-74. (canceled)
